(19) United States
(12) Patent Application Publication
(10) Pub. No.: US 2020/0190043 A1
(43) Pub. Date: Jun. 18, 2020

(54) 2-[[5-(TRIFLUOROMETHYL)-1,2,4-OXADIAZOL-3-YL]ARYLOXY](THIO)ACETAMIDES FOR COMBATING PHYTOPATHOGENIC FUNGI

(71) Applicant: BASF SE, Ludwigshafen am Rhein (DE)

(72) Inventors: Wassilios GRAMMENOS, Ludwigshafen (DE); Violeta TERTERYAN-SEISER, Ludwigshafen (DE); Maria Angelica CRONE-QUINTERO, Limburgerhof (DE); Ian Robert CRAIG, Ludwigshafen (DE); Marcus FEHR, Limburgerhof (DE); Tobias MENTZEL, Limburgerhof (DE); Thomas GROTE, Ludwigshafen (DE); Christine WIEBE, Ludwigshafen (DE); Christian Harald WINTER, Ludwigshafen (DE); Ana ESCRIBANO CUESTA, Ludwigshafen (DE); Bernd MUELLER, Ludwigshafen (DE); Jan Klaas LOHMANN, Ludwigshafen (DE); Michael SEET, Ludwigshafen (DE); Georg Christoph RUDOLF, Ludwigshafen (DE)

(21) Appl. No.: 16/624,047

(22) PCT Filed: Jun. 14, 2018
(86) PCT No.: PCT/EP2018/065770

§ 371 Date: Dec. 18, 2019

(30) Foreign Application Priority Data
Jun. 19, 2017 (EP) 17176620.7
Jul. 7, 2017 (EP) 17180163.2

(51) Int. Cl. C07D 271/06; A01N 43/82; C07C 255/50; C07C 279/18

(52) U.S. Cl. CPC C07D 271/06; C07C 279/18; C07C 255/50; A01N 43/82

(57) ABSTRACT

The present invention relates the compounds of the formula I

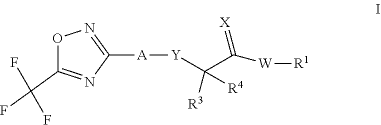

and their N-oxides and agriculturally acceptable salts thereof, and their use for combating phytopathogenic harmful fungi, wherein the variables are defined as given in the description and claims. Further the present invention relates to novel mixtures comprising at least one such compound of the formula I and at least one further pesticidally active substance selected from the group consisting of herbicides, safeners, fungicides, insecticides, and plant growth regulators; and to agrochemical compositions comprising at least one such compound of the formula I and to agrochemical compositions further comprising seeds.

The present invention relates to novel oxadiazoles of the formula I, or an N-oxide, and/or their agriculturally useful salts, to intermediates for their preparation, to their use for controlling phytopathogenic fungi, or to a method for combating phytopathogenic harmful fungi, which process comprises treating the fungi or the materials, plants, the soil or seeds to be protected against fungal attack, with an effective amount of at least one compound of the formula I, or an N-oxide, or an agriculturally acceptable salt thereof; the present invention also relates to mixtures comprising at least one such compound of the formula I and at least one further pesticidally active substance selected from the group consisting of herbicides, safeners, fungicides, insecticides, and plant growth regulators; and to agrochemical compositions comprising at least one such compound of the formula I and to agrochemical compositions further comprising seeds.

WO 2017/076742 inter alia relates to 2-[[5-(trifluoromethyl)-1,2,4-oxadiazol-3-yl]aryl](thio)-acetamide derivatives, wherein X is O or S, and to their use to combat phytopathogenic microorganisms:

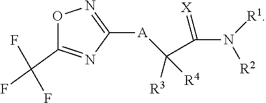

In many cases, in particular at low application rates, the fungicidal activity of known fungicidal compounds is unsatisfactory. Based on this, it was an objective of the present invention to provide compounds having improved activity and/or a broader activity spectrum against phytopathogenic fungi. This objective is achieved by the oxadiazoles of the formula I and/or their agriculturally useful salts for controlling phytopathogenic fungi.

The compounds according to the invention differ from those described in WO 2017/076742 mainly in the introduction of the divalent heteroatom linker group group Y.

Accordingly, the present invention relates to the compounds of the formula I

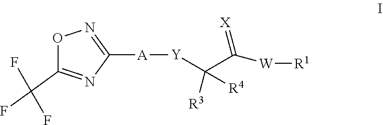

wherein:
A is phenyl or a 5- or 6-membered aromatic heterocycle; wherein the ring member atoms of the aromatic heterocycle include besides carbon atoms 1, 2, 3 or 4 heteroatoms selec¬ted from N, O and S as ring member atoms; and wherein the phenyl ring or the 5- or 6-membered aromatic heterocycle are unsubstituted or substituted with 1, 2, 3 or 4 identical or different groups R^{A}; wherein
   R^{A} is halogen, cyano, diC₁-C₆-alkylamino, C₁-C₆-alkyl, C₁-C₆-alkoxy, C₁-C₆-alkylthio, C₁-C₆-alkylsulfinyl, C₁-C₆-alkylsulfonyl, C₂-C₆-alkenyl, C₂-C₆-alkynyl, C₃-C₁₁-cycloalkyl or C₃-C₁₁-cycloalkoxy; and wherein any of the aliphatic or cyclic moieties are unsubstituted or substituted with 1, 2, 3 or 4 identical or different groups R^{a}; wherein
      R^{a} is halogen, cyano, C₁-C₆-alkyl, C₁-C₆-haloalkyl, C₁-C₆-alkoxy, C₁-C₆-haloalkoxy;
Y is O or S;
R³, R⁴ independently of each other are selected from the group consisting of hydrogen, halogen, cyano, C₁-C₄-alkyl, C₁-C₄-alkenyl, C₁-C₄-alkynyl, C₁-C₄-alkoxy C₁-C₄-haloalkyl and C₁-C₄-haloalkoxy; or
R³ and R⁴ together with the carbon atom to which they are bound form a saturated 3- to 7-membered carbocycle or a saturated 3- to 6-membered heterocycle; wherein the saturated heterocycle includes beside carbon atoms 1, 2 or 3 heteroatoms independently selected from the group consisting of N, O and S as ring member atoms;
   and wherein said N ring member atom is substituted with the group R^{N}; wherein
   R^{N} is hydrogen, C₁-C₆-alkyl or halogen;
   and wherein said S ring member atom is unsubstituted or substituted with 1 or 2 oxo radicals;
   and wherein one or two of the ring member —CH₂— groups of said saturated carbocycle or of said saturated heterocycle may be replaced by one or two groups independently selected from —C(═O)— and —C(═S)—;
   and wherein the carbon ring member atoms of the saturated carbocycle or of the saturated heterocycle are unsubstituted or substituted with a total number of 1, 2, 3, 4 or up to the maximum possible number of identical or different radicals selected from the group consisting of halogen, cyano, C₁-C₆-alkyl, C₁-C₆-alkoxy and C₃-C₁₁-cycloalkyl;
X is O or S;
W is NR², O or S;
R² is hydrogen, CH(═O), C₁-C₆-alkyl, C₂-C₆-alkenyl, C₂-C₆-alkynyl, C₁-C₆-alkoxy, C₃-C₁₁-cycloalkyl, C₃-C₈-cycloalkenyl, C₃-C₁₁-cycloalkyl-C₁-C₄-alkyl, phenyl-C₁-C₄-alkyl, phenyl, pyridinyl, —C(═O)—(C₁-C₆-alkyl), —C(═O)—(C₃-C₁₁-cycloalkyl), —C(═O)—(C₁-C₆-alkoxy) and —N(R^{2a})₂; wherein
   R^{2a} is independently selected from the group consisting of hydrogen, OH, C₁-C₆-alkyl, C₂-C₆-alkenyl, C₂-C₆-alkynyl, C₃-C₁₁-cycloalkyl, C₃-C₈-cycloalkenyl, C₁-C₆-alkoxy, C₁-C₄-alkoxy-C₁-C₄-alkyl and C₁-C₆-alkylthio;
      and wherein any of the aliphatic or cyclic groups in R² are unsubstituted or substituted with 1, 2, 3 or up to the maximum possible number of identical or different radicals selected from the group consisting of halogen, hydroxy, oxo, cyano, C₁-C₆-alkyl, C₁-C₆-alkoxy and C₃-C₁₁-cycloalkyl;
   R¹ is hydrogen, CH(═O), C₁-C₆-alkyl, C₁-C₆-alkoxy, C₃-C₁₁-cycloalkyl, C₃-C₈-cycloalkenyl, C₂-C₆-alkenyl, C₂-C₆-alkynyl, C₁-C₆-alkoxyimino-C₁-C₄-alkyl, C₂-C₆-alkenyloxyimino-C₁-C₄-alkyl, C₂-C₆-alkynyloxyimino-C₁-C₄-alkyl, phenyl-C₁-C₄-alkyl, phenyl-C₁-C₄-alkenyl, phenyl-C₁-C₄-alkynyl, heteroaryl-C₁-C₄-alkyl, phenyl, naphthyl or a 3- to 10-membered saturated, partially unsaturated or aromatic mono- or bicyclic heterocycle, wherein the ring member atoms of said mono- or bicyclic heterocycle include besides carbon atoms further 1, 2, 3 or 4 heteroatoms selected from N, O and S as ring member atoms; and wherein the heteroaryl group in the group heteroaryl-C₁-C₄-alkyl is a 5- or 6-membered aromatic heterocycle, wherein the ring member atoms of the heterocyclic ring include besides carbon atoms 1, 2, 3 or 4 heteroatoms selected from N, O and S as ring member atoms; and wherein any of the above-mentioned aliphatic or cyclic groups are unsubstituted or substituted with 1, 2, 3 or up to the maximum possible number of identical or different groups R^{1a};
   or
if W is NR², R¹ and R², together with the nitrogen atom to which R² is attached, may form a saturated or partially unsaturated mono- or bicyclic 3- to 10-membered heterocycle, wherein the heterocycle includes beside one nitrogen atom and one or more carbon atoms no further heteroatoms or 1, 2 or 3 further heteroatoms independently selected from N, O and S as ring member atoms; and wherein the heterocycle is unsubstituted or substituted with 1, 2, 3, 4 or up to the maximum possible number of identical or different groups R^{1a};
   or
if W is NR² and R² is —N(R^{2a})₂, R¹ and one of the two groups R², together with the nitrogen atom to which said R² is attached, and together with the nitrogen atom to which the group R¹ attached, may form a saturated or partially unsaturated mono- or bicyclic 3- to 10-membered heterocycle, wherein the heterocycle includes beside two nitrogen atoms and one or more carbon atoms no further heteroatoms or 1, 2 or 3 further heteroatoms independently selected from N, O and S as ring member atoms; and wherein the heterocycle is unsubstituted or substituted with 1, 2, 3, 4 or up to the maximum possible number of identical or different groups R^{1a};
   R^{1a} is halogen, oxo, cyano, NO₂, OH, SH, NH₂, C₁-C₆-alkyl, C₂-C₆-alkenyl, C₂-C₆-alkynyl, C₁-C₆-haloalkyl, C₁-C₆-alkoxy, C₁-C₆-haloalkoxy, C₁-C₆-alkylthio, C₁-C₆-haloalkylthio, C₃-C₁₁-cycloalkyl, —NHSO₂—C₁-C₄-alkyl, (C═O)—C₁-C₄-alkyl, —(C═O)—C₁-C₄-alkoxy, C₁-C₆-alkylsulfonyl, hydroxyC₁-C₄-alkyl, —(C═O)—NH₂, —(C═O)—NH(C₁-C₄-alkyl), C₁-C₄-alkylthio-C₁-C₄-alkyl, aminoC₁-C₄-alkyl, C₁-C₄-alkylamino-C₁-C₄-alkyl, diC₁-C₄-alkylamino-C₁-C₄-alkyl, aminocarbonyl-C₁-C₄-alkyl or C₁-C₄-alkoxy-C₁-C₄-alkyl;

or the N-oxides, or the agriculturally acceptable salts thereof, and their use for combating phytopathogenic harmful fungi.

Agriculturally acceptable salts of the compounds of the formula I encompass especially the salts of those cations or the acid addition salts of those acids whose cations and anions, respectively, have no adverse effect on the fungicidal action of the compounds I. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of acceptable acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting a compound I with an acid of the corresponding anion, preferably of hydrochloric acid, hydrobromic acid, sulfuric acid, phosphoric acid or nitric acid.

Compounds of the formula I can exist as one or more stereoisomers. The various stereoisomers include enantiomers, diastereomers, atropisomers arising from restricted rotation about a single bond of asymmetric groups and geometric isomers. They also form part of the subject matter of the present invention. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, e.g. a racemate, individual stereoisomers, or as an optically active form.

Compounds of the formula I can be present in different crystal modifications whose biological activity may differ. They also form part of the subject matter of the present invention.

In respect of the variables, the embodiments of the intermediates obtained during preparation of compounds I correspond to the embodiments of the compounds of formula I. The term “compounds I” refers to compounds of formula I.

In the definitions of the variables given above, collective terms are used which are generally representative for the substituents in question. The term “Cₙ-Cₘ” indicates the number of carbon atoms possible in each case in the substituent or substituent moiety in question.

The moieties having two ore more possibilities to be attached apply following:

The moieties having no brackets in the name are bonded via the last moiety e.g. C₂-C₆-alkenyloxyimino-C₁-C₄-alkyl is bonded via C₁-C₄-alkyl, heteroaryl-C₁-C₄-alkyl is bonded via C₁-C₄-alkyl etc.

The moieties having brackets in the name are bonded via the first moiety e.g. —(C═O)—(C₁-C₆-alkyl) is bonded via C═O, etc.

The term “halogen” refers to fluorine, chlorine, bromine and iodine.

The term “CH(═O)” refers to a formyl group.

The term “C₁-C₆-alkyl” refers to a straight-chained or branched saturated hydrocarbon group having 1 to 6 carbon atoms, for example methyl, ethyl, propyl, 1-methylethyl, butyl, 1-methylpropyl, 2-methylpropyl, and 1,1-dimethylethyl.

The term “C₁-C₆-haloalkyl” refers to a straight-chained or branched alkyl group having 1 to 6 carbon atoms (as defined above), wherein some or all of the hydrogen atoms in these groups may be replaced by halogen atoms as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl.

The term “C₁-C₆-alkoxy” refers to a straight-chain or branched alkyl group having 1 to 6 carbon atoms (as defined above) which is bonded via an oxygen, at any position in the alkyl group, for example methoxy, ethoxy, n-propoxy, 1-methylethoxy, butoxy, 1-methylpropoxy, 2-methylpropoxy or 1,1-dimethylethoxy.

The term “C₁-C₆-haloalkoxy” refers to a C₁-C₆-alkoxy group as defined above, wherein some or all of the hydrogen atoms may be replaced by halogen atoms as mentioned above, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromo-ethoxy, 4-fluorobutoxy, 4-chlorobutoxy, 4-bromobutoxy or nonafluorobutoxy.

The terms “phenyl-C₁-C₄-alkyl or heteroaryl-C₁-C₄-alkyl” refer to alkyl having 1 to 4 carbon atoms (as defined above), wherein one hydrogen atom of the alkyl radical is replaced by a phenyl or hetereoaryl radical respectively such as benzyl.

The term “C₁-C₄-alkoxy-C₁-C₄-alkyl” refers to alkyl having 1 to 4 carbon atoms (as defined above), wherein one hydrogen atom of the alkyl radical is replaced by a C₁-C₄-alkoxy group (as defined above). Likewise, the term “C₁-C₄-alkylthio-C₁-C₄-alkyl” refers to alkyl having 1 to 4 carbon atoms (as defined above), wherein one hydrogen atom of the alkyl radical is replaced by a C₁-C₄-alkylthio group.

The term “C₁-C₆-alkylthio” as used herein refers to straight-chain or branched alkyl groups having 1 to 6 carbon atoms (as defined above) bonded via a sulfur atom. Accordingly, the term “C₁-C₆-haloalkylthio” as used herein refers to straight-chain or branched haloalkyl group having 1 to 6 carbon atoms (as defined above) bonded through a sulfur atom, at any position in the haloalkyl group.

The term “C₁-C₆-alkylsulfinyl” refers to straight-chain or branched alkyl groups having 1 to 6 carbon atoms (as defined above) bonded through a —S(═O)— moiety, at any position in the alkyl group, for example methylsulfinyl and ethylsulfinyl, and the like. Accordingly, the term “C₁-C₆-haloalkylsulfinyl” refers to straight-chain or branched haloalkyl group having 1 to 6 carbon atoms (as defined above), bonded through a —S(═O)— moiety, at any position in the haloalkyl group.

The term “C₁-C₆-alkylsulfonyl” refers to straight-chain or branched alkyl groups having 1 to 6 carbon atoms (as defined above), bonded through a —S(═O)₂— moiety, at any position in the alkyl group, for example methylsulfonyl. Accordingly, the term “C₁-C₆-haloalkylsulfonyl” refers to straight-chain or branched haloalkyl group having 1 to 6 carbon atoms (as defined above), bonded through a —S(═O)₂— moiety, at any position in the haloalkyl group.

The term “hydroxyC₁-C₄-alkyl” refers to alkyl having 1 to 4 carbon atoms, wherein one hydrogen atom of the alkyl radical is replaced by a OH group.

The term “aminoC₁-C₄-alkyl” refers to alkyl having 1 to 4 carbon atoms, wherein one hydrogen atom of the alkyl radical is replaced by a NH₂ group.

The term “C₁-C₄-alkylamino-C₁-C₄-alkyl” refers to refers to alkyl having 1 to 4 carbon atoms (as defined above), wherein one hydrogen atom of the alkyl radical is replaced by a C₁-C₄-alkyl-NH— group which is bound through the nitrogen. Likewise the term “diC₁-C₄-alkylamino-C₁-C₄-alkyl” refers to refers to alkyl having 1 to 4 carbon atoms (as defined above), wherein one hydrogen atom of the alkyl radical is replaced by a (C₁-C₄-alkyl)₂N— group which is bound through the nitrogen.

The term “aminocarbonyl-C₁-C₄-alkyl” refers to alkyl having 1 to 4 carbon atoms, wherein one hydrogen atom of the alkyl radical is replaced by a —(C═O)—NH₂ group.

The term “C₂-C₆-alkenyl” refers to a straight-chain or branched unsaturated hydrocarbon radical having 2 to 6 carbon atoms and a double bond in any position, such as ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₂-C₆-alkynyl” refers to a straight-chain or branched unsaturated hydrocarbon radical having 2 to 6 carbon atoms and containing at least one triple bond, such as ethynyl, 1-propynyl, 2-propynyl (propargyl), 1-butynyl, 2-butynyl, 3-butynyl, 1-methyl-2-propynyl.

The term “C₃-C₁₁-cycloalkyl” refers to a monocyclic, bicyclic or tricyclic saturated univalent hydrocarbon radical having 3 to 11 carbon ring members that is connected through one of the ring carbon atoms by substitution of one hydrogen atom, such as cyclopropyl (C₃H₅), cyclobutyl, cyclopentyl, cyclohexyl, cycloheptyl, cyclooctyl, bicyclo[1.1.0]butyl, bicyclo[2.1.0]pentyl, bicyclo[1.1.1]pentyl, bicyclo[3.1.0]hexyl, bicyclo[2.1.1]hexyl, norcaranyl (bicyclo[4.1.0]heptyl) and norbornyl (bicyclo[2.2.1]heptyl). Further examples of bicyclic or tricyclic cycloalkyl radicals are found herein as examples R¹.1 to R¹.57.

The term “C₃-C₁₁-cycloalkyl-C₁-C₆-alkyl” refers to alkyl having 1 to 6 carbon atoms, wherein one hydrogen atom of the alkyl radical is replaced by a C₃-C₁₁-cycloalkyl group as defined above.

The term “C₃-C₁₁-cycloalkoxy” refers to a cycloalkyl radical having 3 to 11 carbon ring members (as defined above) bonded via an oxygen, for example cyclopropyloxy.

The terms “—C(═O)—C₁-C₄-alkyl”, “—C(═O)—C₁-C₄-alkoxy” and “—C(═O)—C₃-C₁₁-cycloalkyl” refer to radicals which are attached through the carbon atom of the —C(═O)— group.

The term “aliphatic” refers to compounds or radicals composed of carbon and hydrogen and which are non-aromatic compounds. An alicyclic compound or radical is an organic compound that is both aliphatic and cyclic. They contain one or more all-carbon rings which may be either saturated or unsaturated, but do not have aromatic character.

The terms “cyclic moiety” or “cyclic group” refer to a radical which is an alicyclic ring or an aromatic ring, such as, for example, phenyl or heteroaryl.

The term “and wherein any of the aliphatic or cyclic groups are unsubstituted or substituted with 1, 2, 3, 4 or up to the maximum possible number of identical or different groups R^{1a}″ refers to aliphatic groups, cyclic groups and groups, which contain an aliphatic and a cyclic moiety in one group, such as in, for example, phenyl-C₁-C₄-alkyl; therefore a group which contains an aliphatic and a cyclic moiety both of these moieties may be substituted or unsubstituted independently of each other.

The term “heteroaryl” refers to aromatic monocyclic or polycyclic ring systems including besides carbon atoms, 1, 2, 3 or 4 heteroatoms independently selected from the group consisting of N, O and S.

The term “phenyl” refers to an aromatic ring systems including six carbon atoms (commonly referred to as benzene ring). In association with the group A the term “phenyl” is to be interpreted as a benzene ring or phenylene ring, which is attached to both, the oxadiazole moiety and the group L.

The term “saturated or partially unsaturated 3-, 4-, 5-, 6- or 7-membered carbocycle” is to be understood as meaning both saturated or partially unsaturated carbocycles having 3, 4, 5, 6 or 7 ring members. Examples include cyclopropyl, cyclopentyl, cyclopentenyl, cyclopentadienyl, cyclohexyl, cyclohexenyl, cyclohexadienyl, cycloheptyl, cycloheptenyl, cycloheptadienyl, and the like.

The term “3- to 10-membered saturated, partially unsaturated or aromatic mono- or bicyclic heterocycle, wherein the ring member atoms of said mono- or bicyclic heterocycle include besides carbon atoms further 1, 2, 3 or 4 heteroatoms selected from N, O and S as ring member atoms”, is to be understood as meaning both, aromatic mono- and bicyclic heteroaromatic ring systems, and also saturated and partially unsaturated heterocycles, for example:

a 3- or 4-membered saturated heterocycle which contains 1 or 2 heteroatoms from the group consisting of N, O and S as ring members such as oxirane, aziridine, thiirane, oxetane, azetidine, thiethane, [1,2]dioxetane, [1,2]dithietane, [1,2]diazetidine;

and a 5- or 6-membered saturated or partially unsaturated heterocycle which contains 1, 2 or 3 heteroatoms from the group consisting of N, O and S as ring members such as 2-tetrahydro-furanyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydro-pyranyl, 4-tetrahydropyranyl, 2-tetrahydrothienyl, 3-hexahydropyridazinyl, 4-hexa-hydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding-ylidene radicals; and

a 7-membered saturated or partially unsaturated heterocycle such as tetra- and hexahydroazepinyl, such as 2,3,4,5-tetrahydro[1H]azepin-1-,-2-,-3-,-4-,-5-,-6- or-7-yl, 3,4,5,6-tetrahydro[2H]azepin-2-,-3-,-4-,-5-,-6- or-7-yl, 2,3,4,7-tetrahydro[1H]azepin-1-,-2-,-3-,-4-,-5-,-6-or-7-yl, 2,3,6,7-tetrahydro[1H]azepin-1-,-2-,-3-,-4-,-5-,-6- or-7-yl, hexahydroazepin-1-,-2-,-3- or-4-yl, tetra- and hexahydrooxepinyl such as 2,3,4,5-tetrahydro[1H]oxepin-2-,-3-,-4-,-5-,-6- or-7-yl, 2,3,4,7-tetrahydro[1H]oxepin-2-,-3-,-4-,-5-,-6- or-7-yl, 2,3,6,7-tetrahydro[1H]oxepin-2-, -3-,-4-,-5-,-6- or-7-yl, hexahydroazepin-1-,-2-,-3- or-4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding-ylidene radicals; and

the term “5- or 6-membered heteroaryl” or the term “5- or 6-membered aromatic heterocycle” refer to aromatic ring systems including besides carbon atoms, 1, 2, 3 or 4 heteroatoms independently selected from the group consisting of N, O and S, for example, a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl; or

a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

In respect of the variables, the embodiments of the intermediates correspond to the embodiments of the compounds I. Preference is given to those compounds I and, where applicable, also to compounds of all sub-formulae such as 1.1, 1.2 and alike provided herein, wherein all variables have independently of each other or more preferably in combination (any possible combination of 2 or more substituents as defined herein) the following meanings: In one aspect of the invention A is a phenyl, pyridine or thiophene ring; and wherein the cyclic groups A are unsubstituted or substituted with 1, 2, 3 or 4 identical or different groups R^{A} as defined herein; more preferably A is a phenyl wherein the phenyl is unsubstituted or substituted with 1, 2, 3 or 4 identical or different groups R^{A} as defined herein.

In one aspect of the invention A is phenyl which is unsubstituted or substituted with 1, 2, 3 or 4 identical or different groups R^{A} as defined or preferably defined herein and wherein phenyl is attached to the group Y and the oxadiazole moiety as follows (formula I.1):

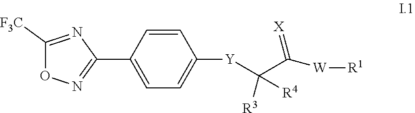

Another embodiment relates to compounds I.1 wherein the phenyl ring is unsubstituted or substituted with 1 or 2 identical or different groups R^{A} as defined or preferably defined herein. A further embodiment relates to compounds I.1 wherein the phenyl ring is unsubstituted. In one aspect of the invention A is a thiophene ring which is unsubstituted or substituted with 1 or 2 identical or different groups R^{A} as defined or preferably defined herein and wherein phenyl is attached to the group Y and the oxadiazole moiety as follows (formula I.2):

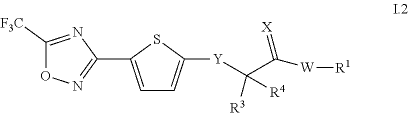

A further embodiment relates to compounds I.2 wherein the thiophene ring is unsubstituted. In one aspect of the invention A is a pyridine ring which is unsubstituted or substituted with 1, 2 or 3 identical or different groups R^{A} as defined or preferably defined herein and wherein phenyl is attached to the group Y and the oxadiazole moiety as follows (formula I.3):

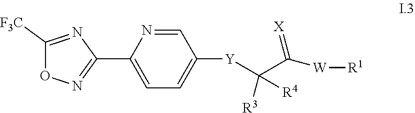

Another embodiment relates to compounds I.3 wherein the pyridine ring is unsubstituted or substituted with 1 or 2 identical or different groups R^{A} as defined or preferably defined herein. A further embodiment relates to compounds I.3 wherein the pyridine ring is unsubstituted. In one aspect of the invention A is a pyridine ring which is unsubstituted or substituted with 1, 2 or 3 identical or different groups R^{A} as defined or preferably defined herein and wherein phenyl is attached to the group Y and the oxadiazole moiety as follows (formula I.4):

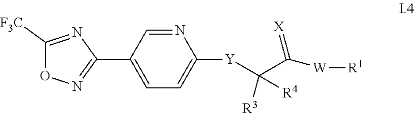

Another embodiment relates to compounds I.4 wherein the pyridine ring is unsubstituted or substituted with 1 or 2 identical or different groups R^{A} as defined or preferably defined herein. A further embodiment relates to compounds I.4 wherein the phenyl ring is unsubstituted.

In a preferred embodiment of the invention R^{A} is halogen, cyano, C₁-C₆-alkyl, C₁-C₆-alkoxy, C₂-C₆-alkenyl, C₂-C₆-alkynyl or C₃-C₈-cycloalkyl; and wherein any of the aliphatic and cyclic moieties are unsubstituted or substituted with 1, 2, 3, 4 or up to the maximum possible number of identical or different groups R^{a} as defined or preferably defined herein.

In another preferred embodiment of the invention R^{A} is halogen, cyano, C₁-C₆-alkyl, C₁-C₆-alkoxy, C₂-C₆-alkenyl, C₂-C₆-alkynyl or C₃-C₈-cycloalkyl; and wherein any of the the aliphatic and cyclic moieties are unsubstituted or substituted with 1, 2, 3, 4 or up to the maximum possible number of identical or different groups selected from halogen, cyano, C₁-C₆-alkyl, C₁-C₆-alkoxy and C₃-C₈-cycloalkyl; in particular fluorine.

More preferably R^{A} is halogen, cyano, C₁-C₆-alkyl, C₁-C₆-haloalkyl, C₁-C₆-alkoxy or C₁-C₆-haloalkoxy; in particular halogen, C₁-C₆-alkyl, C₁-C₆-haloalkyl, C₁-C₆-alkoxy or C₁-C₆-haloalkoxy; more particularly chlorine, fluorine, methyl, methoxy, trifluoromethyl, trifluoromethoxy, difluoromethyl or difluoromethoxy. In a more preferable embodiment R^{A} is chlorine, fluorine or methyl.

R^{a} according to the invention is halogen, cyano, C₁-C₆-alkyl, C₁-C₆-haloalkyl, C₁-C₆-alkoxy, C₁-C₆-haloalkoxy. In a preferred embodiment of the invention R^{a} is halogen, cyano, C₁-C₆-alkyl.

More preferably R^{a} is halogen, in particular fluorine.

In a further embodiment Y is O.

In a further embodiment X is O.

In a further embodiment Y and X are O.

In a further embodiment W is NR².

In a further embodiment W is O or S, in particular O.

Further embodiments 1.1 to 1.10 of the present invention relate to compounds I, wherein group R¹ is defined as follows:

### Embodiment 1.1

R¹ is CH(═O), C₁-C₆-alkyl, C₂-C₆-alkenyl, C₂-C₆-alkynyl, C₃-C₈-cycloalkyl, C₃-C₈-cycloalkenyl, phenyl-C₁-C₄-alkyl, heteroaryl-C₁-C₄-alkyl, phenyl or heteroaryl; and wherein the heteroaryl group is a 5- or 6-membered aromatic heterocycle, wherein the ring includes besides carbon atoms 1, 2, 3 or 4 heteroatoms selected from N, O and S as ring member atoms; and wherein any of the aliphatic or cyclic groups are unsubstituted or substituted with 1, 2, 3 or up to the maximum possible number of identical or different radicals R^{1a} as defined or preferably defined herein.

### Embodiment 1.2

R¹ is phenyl, benzyl or heteroaryl; and wherein the heteroaryl group is a 5- or 6-membered aromatic heterocycle, wherein the ring includes besides carbon atoms 1, 2, 3 or 4 heteroatoms selected from N, O and S as ring member atoms; and wherein any of the cyclic groups are unsubstituted or substituted with 1, 2, 3 or up to the maximum possible number of identical or different radicals R^{1a} as defined or preferably defined herein.

### Embodiment 1.3

R¹ is C₃-C₈-cycloalkyl or C₃-C₈-cycloalkenyl; and wherein the cyclic group is unsubstituted or substituted with 1, 2, 3 or up to the maximum possible number of identical or different radicals R^{1a} as defined or preferably defined herein.

### Embodiment 1.4

R¹ is C₁-C₆-alkyl; and wherein the alkyl group is unsubstituted or substituted with 1, 2, 3 or up to the maximum possible number of identical or different radicals R^{1a} as defined or preferably defined herein.

### Embodiment 1.5

R¹ is difluoromethyl, trifluoromethyl, 2,2-difluoroethyl, 2,2,2-trifluoroethyl, 2-chloro-2-fluoroethyl, 2-chloro-2,2-difluoroethyl, 2,2,2-trichloroethyl and pentafluoroethyl, 3,3,3-trifluoropropyl, CH₂CF₂CF₃ or CF₂CF₂CF₅, CH(CH₃)CF₃, CH₂CF₂CH₃, CH₂C(CH₃)₂F, CH₂CH(CH₃)CF₃ or CH₂C(CH₃)₂CF₃.

### Embodiment 1.6

R¹ is C₁-C₆-alkoxyimino-C₁-C₄-alkyl, C₂-C₆-alkenyloxyimino-C₁-C₄-alkyl or C₂-C₆-alkynyloxyimino-C₁-C₄-alkyl.

### Embodiment 1.7

R¹ is a bicyclic or tricyclic C₄-C₁₁-cycloalkyl which is unsubstituted or substituted with 1, 2 or 3 radicals selected from the group consisting of oxo, hydroxy, halogen and C₁-C₃-alkyl.

### Embodiment 1.8

R¹ is a bicyclic or tricyclic carbocycle selected from the group consisting of radicals R¹.1 to R¹.31 below; wherein each radical may be connected to the remainder of the compounds of formula I through one of the ring carbon atoms by substitution of one hydrogen atom; and wherein R¹ is unsubstituted or substituted with 1, 2 or 3 radicals selected from the group consisting of oxo, hydroxy, halogen and C₁-C₃-alkyl.

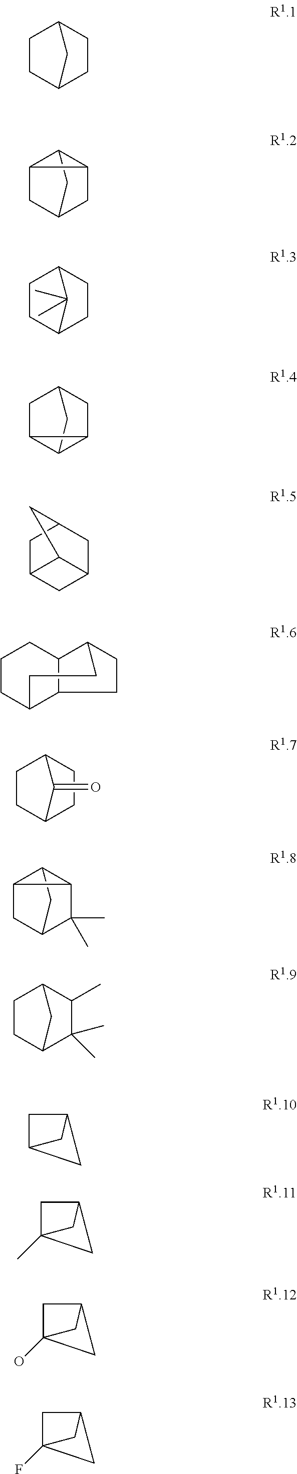

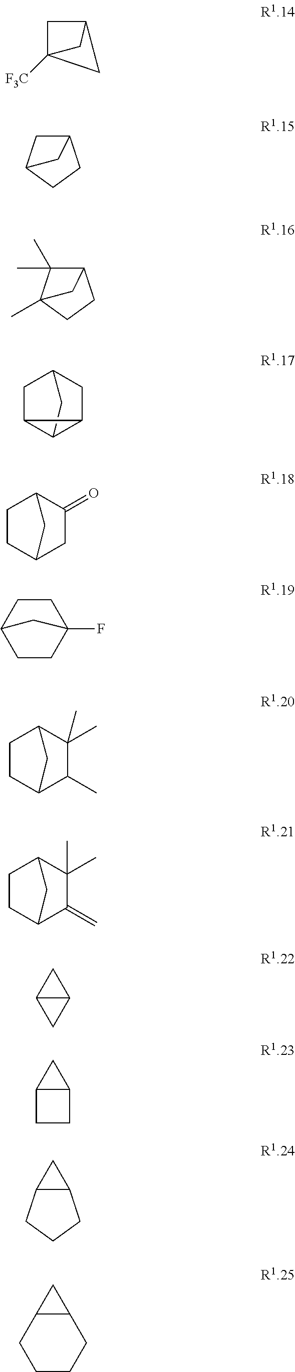

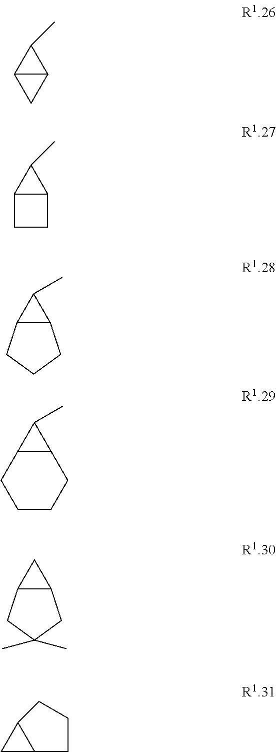

### Embodiment 1.9

R¹ is selected from the group consisting of R¹.32 to R¹.57 below, particularly from R¹.32 to R¹.49, which are further unsubstituted, and wherein “#C” indicates the carbon atom, which is attached to the remainder of the compounds of formula I.

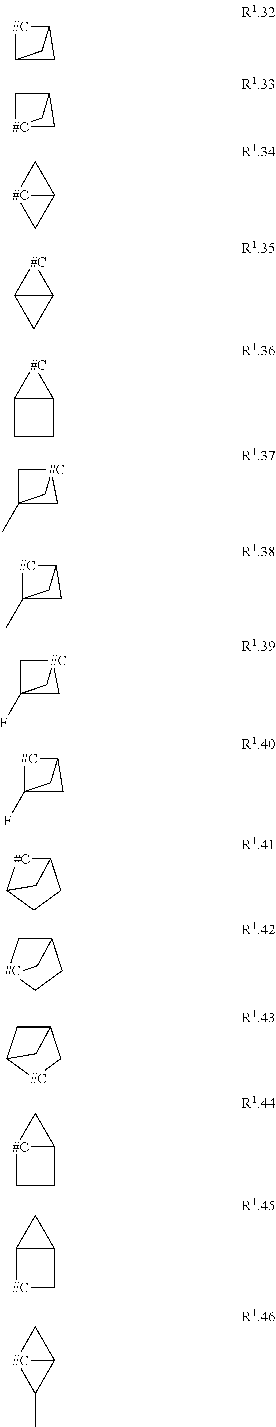

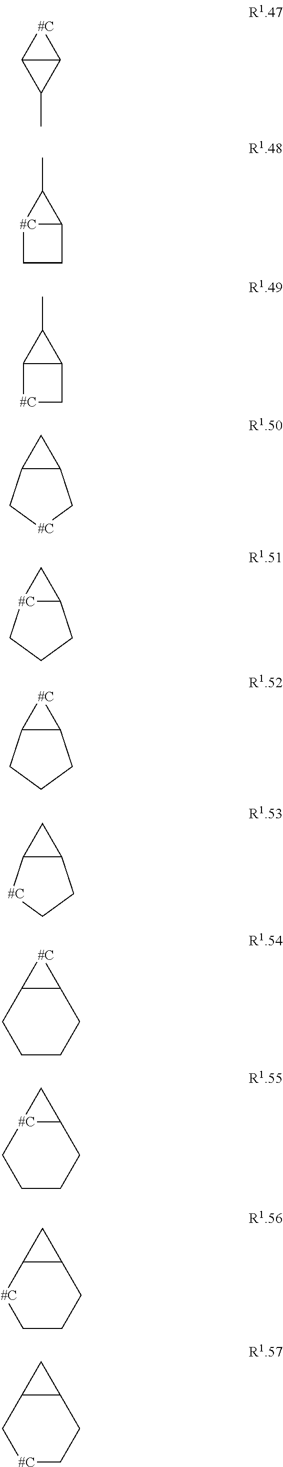

### Embodiment 1.10

R¹ is CH(═O).

### Embodiment 1.11

R¹ is hydrogen.

Further embodiments of the present invention relate to compounds I, wherein group R^{1a} is defined as follows: In one embodiment R^{1a} is selected from the group consisting of halogen, cyano, C₁-C₆-alkyl, C₁-C₆-haloalkyl, C₁-C₆-alkoxy, C₁-C₆-haloalkoxy and C₃-C₈-cycloalkyl.

In another aspect of the invention R^{1a} is selected from the group consisting of fluorine, chlorine, cyano, methyl, ethyl, methoxy, trifluoromethyl, trifluoromethoxy, difluoromethyl, difluoromethoxy or cyclopropyl.

In another embodiment R^{1a} is selected from the group consisting of halogen, C₁-C₆-alkyl and C₃-C₈-cycloalkyl; particularly from methyl, ethyl, fluorine and chlorine; more particularly from fluorine and chlorine.

According to a further embodiment R¹ is C₁-C₆-alkyl, C₁-C₆-alkoxy, C₃-C₆-cycloalkyl or a 4- to 5-membered saturated or partially unsaturated heterocycle, wherein the ring member atoms of said heterocycle include besides carbon atoms 1 or 2 heteroatoms selected from N and O as ring member atoms; and wherein any of the above-mentioned aliphatic or cyclic groups R¹ are unsubstituted or substituted with 1, 2 or 3 of identical or different groups R^{1a}; wherein R^{1a} is halogen, oxo, cyano, NO₂, OH, C₁-C₆-alkyl, C₁-C₆-haloalkyl, C₁-C₆-alkoxy, C₁-C₆-haloalkoxy or C₃-C₈-cycloalkyl.

In another embodiment R² is hydrogen, CH(═O), C₁-C₆-alkyl, C₁-C₆-alkoxy, C₂-C₆-alkenyl, propargyl, C₃-C₈-cycloalkyl, C₃-C₈-cycloalkenyl, C₃-C₈-cycloalkyl-C₁-C₄-alkyl, phenyl, pyridinyl or —N(R^{2a})₂; and wherein any of the aliphatic or cyclic groups are unsubstituted or substituted with 1, 2, 3, 4 or up to the maximum possible number of identical or different radicals selected from the group consisting of halogen, cyano, C₁-C₆-alkyl and C₁-C₆-alkoxy; more preferably from halogen, in particular the radical is fluorine; and wherein R² is independently selected from the group consisting of hydrogen, OH, C₁-C₆-alkyl, C₂-C₆-alkenyl, C₂-C₆-alkynyl, C₃-C₈-cycloalkyl or C₁-C₆-alkoxy.

Further embodiments 2.1 to 2.8 relate to compounds I, wherein group R² is defined as follows:

### Embodiment 2.1

R² is hydrogen, CH(═O), C₁-C₆-alkyl, C₁-C₆-alkoxy, C₂-C₆-alkenyl, propargyl, C₃-C₈-cycloalkyl, C₃-C₈-cycloalkenyl, C₃-C₈-cycloalkyl-C₁-C₄-alkyl, phenyl, C₁-C₆-alkylamino or diC₁-C₆-alkylamino; and wherein any of the aliphatic or cyclic groups are unsubstituted or substituted with 1, 2, 3, 4 or up to the maximum possible number of identical or different radicals selected from the group consisting of halogen, cyano, C₁-C₆-alkyl and C₁-C₆-alkoxy.

### Embodiment 2.2

R² is hydrogen, methyl, ethyl, n-propyl, iso-propyl, methoxy, ethyoxy, propyloxy, cyclopropyl, cyclopropyl-CH₂—, allyl, phenyl, 4-F-phenyl, 2-F-phenyl, C₁-C₆-alkylamino or diC₁-C₆-alkylamino.

### Embodiment 2.3

R² is hydrogen, C₁-C₆-alkyl, C₁-C₆-alkoxy, C₂-C₆-alkenyl, propargyl, C₃-C₈-cycloalkyl, C₁-C₆-alkylamino or diC₁-C₆-alkylamino.

### Embodiment 2.4

R² is hydrogen, methyl, ethyl, n-propyl, iso-propyl, methoxy, ethyoxy, propyloxy, cyclopropyl, cyclopropyl-CH₂—, allyl, C₁-C₆-alkylamino or diC₁-C₆-alkylamino.

### Embodiment 2.5

R² is hydrogen, C₁-C₆-alkyl, C₁-C₆-alkoxy, C₂-C₆-alkenyl or propargyl, C₁-C₆-alkylamino or diC₁-C₆-alkylamino.

### Embodiment 2.6

R² is hydrogen, methy, ethyl, methoxy, ethyoxy, propyloxy, C₁-C₆-alkylamino or diC₁-C₆-alkylamino.

### Embodiment 2.7

R² is CH(═O).

### Embodiment 2.8

R² is hydrogen.

According to a further embodiment, R² is CH(═O), C₁-C₄-alkyl, C₁-C₆-alkoxy or C₃-C₈-cycloalkyl, and wherein any of the aliphatic or cyclic groups in R² are unsubstituted or substituted with 1, 2, 3 or up to the maximum possible number of identical or different radicals selected from halogen.

In one embodiment R² is hydrogen, CH(═O), C₁-C₆-alkyl, C₁-C₆-alkoxy, C₂-C₆-alkenyl, propargyl, C₃-C₈-cycloalkyl, C₃-C₈-cycloalkenyl, C₃-C₈-cycloalkyl-C₁-C₄-alkyl, phenyl, pyridinyl or —N(R^{2a})₂; and wherein any of the aliphatic or cyclic groups are unsubstituted or substituted with 1, 2, 3, 4 or up to the maximum possible number of identical or different radicals selected from the group consisting of halogen, cyano, C₁-C₆-alkyl and C₁-C₆-alkoxy; more preferably from halogen, in particular the radical is fluorine; and wherein R^{2a} is independently selected from the group consisting of hydrogen, OH, C₁-C₆-alkyl, C₂-C₆-alkenyl, C₂-C₆-alkynyl, C₃-C₈-cycloalkyl or C₁-C₆-alkoxy; and R¹ is C₁-C₆-alkyl, C₃-C₈-cycloalkyl, C₃-C₈-cycloalkenyl, C₂-C₆-alkenyl, C₂-C₆-alkynyl, phenyl-C₁-C₄-alkyl, heteroaryl-C₁-C₄-alkyl, phenyl or heteroaryl; and wherein the heteroaryl group is a 5- or 6-membered aromatic heterocycle, wherein the ring includes besides carbon atoms 1, 2, 3 or 4 heteroatoms selected from N, O and S as ring member atoms; and wherein any of the aliphatic or cyclic groups in R¹ are unsubstituted or substituted with 1, 2, 3, 4 or up to the maximum possible number of identical or different radicals R^{1a} as defined or preferably defined herein.

In another aspect R² is hydrogen, C₁-C₆-alkyl, C₁-C₆-alkoxy, C₂-C₆-alkenyl, propargyl, C₃-C₈-cycloalkyl, C₃-C₈-cycloalkenyl, C₃-C₈-cycloalkyl-C₁-C₄-alkyl, C₁-C₆-alkylamino or diC₁-C₆-alkylamino; and R¹ is C₁-C₆-alkyl, C₃-C₈-cycloalkyl, C₃-C₈-cycloalkenyl, C₂-C₆-alkenyl, C₂-C₆-alkynyl, phenyl-C₁-C₄-alkyl, heteroaryl-C₁-C₄-alkyl, phenyl or heteroaryl; and wherein the heteroaryl group is a 5- or 6-membered aromatic heterocycle, wherein the ring includes besides carbon atoms 1, 2, 3 or 4 heteroatoms selected from N, O and S as ring member atoms; and wherein any of the aliphatic or cyclic groups in R¹ are unsubstituted or substituted with 1, 2, 3, 4 or up to the maximum possible number of identical or different radicals R^{1a} as defined or preferably defined herein.

In another aspect R² is hydrogen, CH(═O), methyl, ethyl, n-propyl, iso-propyl, methoxy, ethyoxy, propyloxy, cyclopropyl, cyclopropyl-CH₂—, allyl, C₁-C₆-alkylamino or diC₁-C₆-alkylamino; and R¹ is C₁-C₆-alkyl, C₃-C₈-cycloalkyl, C₃-C₈-cycloalkenyl, C₂-C₆-alkenyl or C₂-C₆-alkynyl; and wherein any of the aliphatic or cyclic groups in R¹ are unsubstituted or substituted with 1, 2, 3, 4 or up to the maximum possible number of identical or different radicals selected from the group consisting of halogen or C₁-C₆-alkyl.

In another aspect R² is hydrogen, CH(═O), methyl, ethyl, methoxy, ethyoxy, cyclopropyl or cyclopropyl-CH₂—; and R¹ is hydrogen, CH(═O), methyl, ethyl, methoxy, ethyoxy, cyclopropyl or cyclopropyl-CH₂—; and wherein any of the aliphatic or cyclic groups in R¹ and/or R² are unsubstituted or substituted with 1, 2, 3, 4 or up to the maximum possible number of identical or different halogen radicals.

In another aspect, W is NR², R² is hydrogen, CH(═O), methyl, ethyl, methoxy, ethyoxy, cyclopropyl or cyclopropyl-CH₂—; and R¹ is hydrogen, CH(═O), methyl, ethyl, methoxy, ethyoxy, cyclopropyl or cyclopropyl-CH₂—; and wherein any of the aliphatic or cyclic groups in R¹ and/or R² are unsubstituted or substituted with 1, 2, 3, 4 or up to the maximum possible number of identical or different halogen radicals.

Further embodiments 3.1 to 3.15 of the present invention relate to compounds I, wherein groups R³ and R⁴ are defined as follows: Embodiment 3.1: R³ and R⁴ independently of each other are hydrogen, halogen, C₁-C₆-alkyl or C₁-C₆-haloalkyl; or R³ and R⁴ together with the carbon atom to which they are bound form a cyclopropyl ring, wherein the cyclopropyl ring is unsubstituted.

### Embodiment 3.2

R³ and R⁴ independently of each other are hydrogen or C₁-C₄-alkyl;

### Embodiment 3.3

R³ and R⁴ independently of each other are hydrogen, methyl or ethyl.

### Embodiment 3.4

R³ and R⁴ are independently of each other hydrogen, fluorine, chlorine, methyl or trifluoromethyl; or R³ and R⁴ together with the carbon atom to which they are bound form a cyclopropyl ring, wherein the cyclopropyl ring is unsubstituted.

### Embodiment 3.5

R³ and R⁴ are both hydrogen.

### Embodiment 3.6

R³ is hydrogen and R⁴ is methyl.

### Embodiment 3.7

R³ and R⁴ are both methyl.

### Embodiment 3.8

R³ and R⁴ are both fluorine.

### Embodiment 3.9

R³ and R⁴ are both trifluoromethyl.

### Embodiment 3.10

R³ and R⁴ together with the carbon atom to which they are bound a saturated monocyclic 3- to 5-membered saturated heterocycle or saturated carbocycle; and wherein the saturated heterocycle includes beside one or more carbon atoms no heteroatoms or 1 or 2 heteroatoms independently selected from N, O and S as ring member atoms; and wherein the heterocycle or the carbocycle is unsubstituted or substituted 1, 2, 3, 4 or up to the maximum possible number of identical or different radicals selected from the group consisting of halogen, cyano and C₁-C₂-alkyl.

### Embodiment 3.11

R³ and R⁴ together with the carbon atom to which they are bound form a 3- or 4-membered carbocylic ring; and wherein the carbocylic ring is unsubstituted.

### Embodiment 3.12

R³ and R⁴ together with the carbon atom to which they are bound form a cyclopropyl ring, wherein the cyclopropyl ring is unsubstituted.

### Embodiment 3.13

R³ and R⁴ together with the carbon atom to which they are bound form a saturated 3-membered heterocycle; wherein the heterocycle includes beside two carbon atoms one heteroatom selected from N, O and S as ring member atoms; and wherein the heterocycle is unsubstituted or substituted 1, 2, 3, 4 or up to the maximum possible number of identical or different radicals selected from the group consisting of halogen, cyano and C₁-C₂-alkyl.

### Embodiment 3.14

R³ is methyl and R⁴ is fluorine.

### Embodiment 3.15

R³ is hydrogen and R⁴ is fluorine.

In a further embodiment the invention relates to compounds of the formula I.1 and their use for combating phytopathogenic harmful fungi,

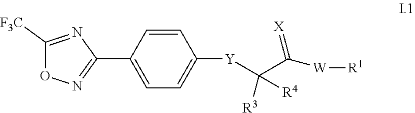

wherein:
Y is O;
   R³, R⁴ independently of each other are selected from the group consisting of hydrogen, fluorine, methyl, trifluoromethyl, OCHF₂, or
      R³ and R⁴ together with the carbon atom to which they are bound form a cyclopropyl ring;
X is O;
W is NR²;
   R² is C₁-C₆-alkyl, CH(═O), C₁-C₆-alkoxy or C₃-C₈-cycloalkyl, and wherein any of the aliphatic or cyclic groups are unsubstituted or substituted with 1, 2, 3, 4 or up to the maximum possible number of identical or different radicals selected from the group consisting of halogen or cyano;
R¹ is hydrogen, C₁-C₆-alkyl, C₁-C₆-alkoxy, C₃-C₈-cycloalkyl, phenyl or benzyl, and wherein any of the aliphatic or cyclic groups are unsubstituted or substituted with 1, 2 or 3 of identical or different groups R^{1a}; wherein R^{1a} is selected from fluorine, chlorine, cyano, methyl, ethyl, methoxy, trifluoromethyl, trifluoromethoxy, difluoromethyl, difluoromethoxy and cyclopropyl.

In a further embodiment the invention relates to compounds of the formula I.1 and their use for combating phytopathogenic harmful fungi, wherein:
Y is O;
R³, R⁴ are both fluorine;
X is O or S;
W is NR²;
   R² is hydrogen, C₁-C₆-alkyl, CH(═O), C₁-C₆-alkoxy or C₃-C₈-cycloalkyl, and wherein any of the aliphatic or cyclic groups are unsubstituted or substituted with 1, 2, 3, 4 or up to the maximum possible number of identical or different radicals selected from the group consisting of halogen or cyano;
R¹ is hydrogen, C₁-C₆-alkyl, C₁-C₆-alkoxy, C₃-C₈-cycloalkyl, phenyl or benzyl, and wherein any of the aliphatic or cyclic groups are unsubstituted or substituted with 1, 2 or 3 of identical or different groups R^{1a}; wherein R^{1a} is selected from fluorine, chlorine, cyano, methyl, ethyl, methoxy, trifluoromethyl, trifluoromethoxy, difluoromethyl, difluoromethoxy and cyclopropyl.

In a further embodiment the invention relates to compounds of the formula I.1 and their use for combating phytopathogenic harmful fungi, wherein:
Y is O;
R³, R⁴ are both fluorine;
X is O or S;
W is NR²;
   R² is hydrogen, methyl, ethyl, n-propyl, iso-propyl, methoxy, ethyoxy, propyloxy, cyclopropyl, cyclopropyl-CH₂—, allyl;
R¹ is hydrogen, C₁-C₆-alkyl, C₃-C₈-cycloalkyl, C₃-C₈-cycloalkenyl, C₂-C₆-alkenyl or C₂-C₆-alkynyl; and wherein any of the aliphatic or cyclic groups in R¹ are unsubstituted or substituted with 1, 2, 3, 4 or up to the maximum possible number of identical or different radicals selected from the group consisting of halogen or C₁-C₆-alkyl.

In a further embodiment the invention relates to compounds of the formula I.1 and their use for combating phytopathogenic harmful fungi, wherein:
Y is O;
R³, R⁴ are both fluorine;
X is O or S;
W is NR²;
   R² is hydrogen, methyl or ethyl;
R¹ is hydrogen, C₁-C₆-alkyl, C₃-C₈-cycloalkyl; in particular hydrogen, methyl, ethyl, n-propyl, iso-propyl and cyclopropyl.

Preference is given to the compounds I according to the invention compiled in Tables 1 to 90 below, each of which compiles 56 different compounds I. The groups mentioned for a substituent in the tables are furthermore per se, independently of the combination in which they are mentioned, a particularly preferred aspect of the substituent in question.

Table 1: Compounds of the formula I.1, in which Y and X are both O, R³ and R⁴ are both hydrogen, W is NH, and R¹ for a compound corresponds in each case to one row of Table A.

Table 2: Compounds of the formula I.1, in which Y and X are both O, R³ and R⁴ are both hydrogen, W is NCH₃, and R¹ for a compound corresponds in each case to one row of Table A.

Table 3: Compounds of the formula I.1, in which Y and X are both O, R³ and R⁴ are both hydrogen, W is NC₂H₅, and R¹ for a compound corresponds in each case to one row of Table A.

Table 4: Compounds of the formula I.1, in which Y and X are both O, R³ and R⁴ are both hydrogen, W is N—CH(═O), and R¹ for a compound corresponds in each case to one row of Table A.

Table 5: Compounds of the formula I.1, in which Y and X are both S, R³ and R⁴ are both hydrogen, W is NH, and R¹ for a compound corresponds in each case to one row of Table A.

Table 6: Compounds of the formula I.1, in which Y and X are both S, R³ and R⁴ are both hydrogen, W is NCH₃, and R¹ for a compound corresponds in each case to one row of Table A.

Table 7: Compounds of the formula I.1, in which Y and X are both S, R³ and R⁴ are both hydrogen, W is NC₂H₅, and R¹ for a compound corresponds in each case to one row of Table A.

Table 8: Compounds of the formula I.1, in which Y and X are both S, R³ and R⁴ are both hydrogen, W is N—CH(═O), and R¹ for a compound corresponds in each case to one row of Table A.

Table 9: Compounds of the formula I.1, in which Y is O and X is S, R³ and R⁴ are both hydrogen, W is NH, and R¹ for a compound corresponds in each case to one row of Table A.

Table 10: Compounds of the formula I.1, in which Y is O and X is S, R³ and R⁴ are both hydrogen, W is NCH₃, and R¹ for a compound corresponds in each case to one row of Table A.

Table 11: Compounds of the formula I.1, in which Y is O and X is S, R³ and R⁴ are both hydrogen, W is NC₂H₅, and R¹ for a compound corresponds in each case to one row of Table A.

Table 12: Compounds of the formula I.1, in which Y is O and X is S, R³ and R⁴ are both hydrogen, W is N—CH(═O), and R¹ for a compound corresponds in each case to one row of Table A.

Table 13: Compounds of the formula I.1, in which Y is S and X is O, R³ and R⁴ are both hydrogen, W is NH, and R¹ for a compound corresponds in each case to one row of Table A.

Table 14: Compounds of the formula I.1, in which Y is S and X is O, R³ and R⁴ are both hydrogen, W is NCH₃, and R¹ for a compound corresponds in each case to one row of Table A.

Table 15: Compounds of the formula I.1, in which Y is S and X is O, R³ and R⁴ are both hydrogen, W is NC₂H₅, and R¹ for a compound corresponds in each case to one row of Table A.

Table 16: Compounds of the formula I.1, in which Y is S and X is O, R³ and R⁴ are both hydrogen, W is N—CH(═O), and R¹ for a compound corresponds in each case to one row of Table A.

Table 17: Compounds of the formula I.1, in which Y and X are both O, R³ and R⁴ are both F, W is NH, and R¹ for a compound corresponds in each case to one row of Table A.

Table 18: Compounds of the formula I.1, in which Y and X are both O, R³ and R⁴ are both F, W is NCH₃, and R¹ for a compound corresponds in each case to one row of Table A.

Table 19: Compounds of the formula I.1, in which Y and X are both O, R³ and R⁴ are both F, W is NC₂H₅, and R¹ for a compound corresponds in each case to one row of Table A.

Table 20: Compounds of the formula I.1, in which Y and X are both O, R³ and R⁴ are both F, W is NCH(═O), and R¹ for a compound corresponds in each case to one row of Table A.

Table 21: Compounds of the formula I.1, in which Y and X are both S, R³ and R⁴ are both F, W is NH, and R¹ for a compound corresponds in each case to one row of Table A.

Table 22: Compounds of the formula I.1, in which Y and X are both S, R³ and R⁴ are both F, W is NCH₃, and R¹ for a compound corresponds in each case to one row of Table A.

Table 23: Compounds of the formula I.1, in which Y and X are both S, R³ and R⁴ are both F, W is NC₂H₅, and R¹ for a compound corresponds in each case to one row of Table A.

Table 24: Compounds of the formula I.1, in which Y and X are both S, R³ and R⁴ are both F, W is N—CH(═O), and R¹ for a compound corresponds in each case to one row of Table A.

Table 25: Compounds of the formula I.1, in which Y is O and X is S, R³ and R⁴ are both F, W is NH, and R¹ for a compound corresponds in each case to one row of Table A.

Table 26: Compounds of the formula I.1, in which Y is O and X is S, R³ and R⁴ are both F, W is NCH₃, and R¹ for a compound corresponds in each case to one row of Table A.

Table 27: Compounds of the formula I.1, in which Y is O and X is S, R³ and R⁴ are both F, W is NC₂H₅, and R¹ for a compound corresponds in each case to one row of Table A.

Table 28: Compounds of the formula I.1, in which Y is O and X is S, R³ and R⁴ are both F, W is N—CH(═O), and R¹ for a compound corresponds in each case to one row of Table A.

Table 29: Compounds of the formula I.1, in which Y is S and X is O, R³ and R⁴ are both F, W is NH, and R¹ for a compound corresponds in each case to one row of Table A.

Table 30: Compounds of the formula I.1, in which Y is S and X is O, R³ and R⁴ are both F, W is NCH₃, and R¹ for a compound corresponds in each case to one row of Table A.

Table 31: Compounds of the formula I.1, in which Y is S and X is O, R³ and R⁴ are both F, W is NC₂H₅, and R¹ for a compound corresponds in each case to one row of Table A.

Table 32: Compounds of the formula I.1, in which Y is S and X is O, R³ and R⁴ are both F, W is N—CH(═O), and R¹ for a compound corresponds in each case to one row of Table A.

Table 33: Compounds of the formula I.1, in which Y and X are both O, R³ and R⁴ are both CH₃, W is NH, and R¹ for a compound corresponds in each case to one row of Table A.

Table 34: Compounds of the formula I.1, in which Y and X are both O, R³ and R⁴ are both CH₃, W is NCH₃, and R¹ for a compound corresponds in each case to one row of Table A.

Table 35: Compounds of the formula I.1, in which Y and X are both O, R³ and R⁴ are both CH₃, W is NC₂H₅, and R¹ for a compound corresponds in each case to one row of Table A.

Table 36: Compounds of the formula I.1, in which Y and X are both O, R³ and R⁴ are both CH₃, W is N—CH(═O), and R¹ for a compound corresponds in each case to one row of Table A.

Table 37: Compounds of the formula I.1, in which Y and X are both S, R³ and R⁴ are both CH₃, W is NH, and R¹ for a compound corresponds in each case to one row of Table A.

Table 38: Compounds of the formula I.1, in which Y and X are both S, R³ and R⁴ are both CH₃, W is NCH₃, and R¹ for a compound corresponds in each case to one row of Table A.

Table 39: Compounds of the formula I.1, in which Y and X are both S, R³ and R⁴ are both CH₃, W is NC₂H₅, and R¹ for a compound corresponds in each case to one row of Table A.

Table 40: Compounds of the formula I.1, in which Y and X are both S, R³ and R⁴ are both CH₃, W is N—CH(═O), and R¹ for a compound corresponds in each case to one row of Table A.

Table 41: Compounds of the formula I.1, in which Y is O and X is S, R³ and R⁴ are both CH₃, W is NH, and R¹ for a compound corresponds in each case to one row of Table A.

Table 42: Compounds of the formula I.1, in which Y is O and X is S, R³ and R⁴ are both CH₃, W is NCH₃, and R¹ for a compound corresponds in each case to one row of Table A.

Table 43: Compounds of the formula I.1, in which Y is O and X is S, R³ and R⁴ are both CH₃, W is NC₂H₅, and R¹ for a compound corresponds in each case to one row of Table A.

Table 44: Compounds of the formula I.1, in which Y is O and X is S, R³ and R⁴ are both CH₃, W is N—CH(═O), and R¹ for a compound corresponds in each case to one row of Table A.

Table 45: Compounds of the formula I.1, in which Y is S and X is O, R³ and R⁴ are both CH₃, W is NH, and R¹ for a compound corresponds in each case to one row of Table A.

Table 46: Compounds of the formula I.1, in which Y is S and X is O, R³ and R⁴ are both CH₃, W is NCH₃, and R¹ for a compound corresponds in each case to one row of Table A.

Table 47: Compounds of the formula I.1, in which Y is S and X is O, R³ and R⁴ are both CH₃, W is NC₂H₅, and R¹ for a compound corresponds in each case to one row of Table A.

Table 48: Compounds of the formula I.1, in which Y is S and X is O, R³ and R⁴ are both CH₃, W is N—CH(═O), and R¹ for a compound corresponds in each case to one row of Table A.

Table 49: Compounds of the formula I.1, in which Y and X are both O, R³ is hydrogen, R⁴ is CH₃, W is NH, and R¹ for a compound corresponds in each case to one row of Table A.

Table 50: Compounds of the formula I.1, in which Y and X are both O, R³ is hydrogen, R⁴ is CH₃, W is NCH₃, and R¹ for a compound corresponds in each case to one row of Table A.

Table 51: Compounds of the formula I.1, in which Y and X are both O, R³ is hydrogen, R⁴ is CH₃, W is NC₂H₅, and R¹ for a compound corresponds in each case to one row of Table A.

Table 52: Compounds of the formula I.1, in which Y and X are both O, R³ is hydrogen, R⁴ is CH₃, W is N—CH(═O), and R¹ for a compound corresponds in each case to one row of Table A.

Table 53: Compounds of the formula I.1, in which Y and X are both S, R³ is hydrogen, R⁴ is CH₃, W is NH, and R¹ for a compound corresponds in each case to one row of Table A.

Table 54: Compounds of the formula I.1, in which Y and X are both S, R³ is hydrogen, R⁴ is CH₃, W is NCH₃, and R¹ for a compound corresponds in each case to one row of Table A.

Table 55: Compounds of the formula I.1, in which Y and X are both S, R³ is hydrogen, R⁴ is CH₃, W is NC₂H₅, and R¹ for a compound corresponds in each case to one row of Table A.

Table 56: Compounds of the formula I.1, in which Y and X are both S, R³ is hydrogen, R⁴ is CH₃, W is N—CH(═O), and R¹ for a compound corresponds in each case to one row of Table A.

Table 57: Compounds of the formula I.1, in which Y is O and X is S, R³ is hydrogen, R⁴ is CH₃, and R¹ for a compound corresponds in each case to one row of Table A.

Table 58: Compounds of the formula I.1, in which Y is O and X is S, R³ is hydrogen, R⁴ is CH₃, W is NCH₃, and R¹ for a compound corresponds in each case to one row of Table A.

Table 59: Compounds of the formula I.1, in which Y is O and X is S, R³ is hydrogen, R⁴ is CH₃, W is NC₂H₅, and R¹ for a compound corresponds in each case to one row of Table A.

Table 60: Compounds of the formula I.1, in which Y is O and X is S, R³ is hydrogen, R⁴ is CH₃, W is N—CH(═O), and R¹ for a compound corresponds in each case to one row of Table A.

Table 61: Compounds of the formula I.1, in which Y is S and X is O, R³ is hydrogen, R⁴ is CH₃, W is NH, and R¹ for a compound corresponds in each case to one row of Table A.

Table 62: Compounds of the formula I.1, in which Y is S and X is O, R³ is hydrogen, R⁴ is CH₃, W is NCH₃, and R¹ for a compound corresponds in each case to one row of Table A.

Table 63: Compounds of the formula I.1, in which Y is S and X is O, R³ is hydrogen, R⁴ is CH₃, W is NC₂H₅, and R¹ for a compound corresponds in each case to one row of Table A.

Table 64: Compounds of the formula I.1, in which Y is S and X is O, R³ is hydrogen, R⁴ is CH₃, W is N—CH(═O), and R¹ for a compound corresponds in each case to one row of Table A.

Table 65: Compounds of the formula I.1, in which Y and X are both O, R³ and R⁴ are both cyclopropyl, W is NH, and R¹ for a compound corresponds in each case to one row of Table A.

Table 66: Compounds of the formula I.1, in which Y and X are both O, R³ and R⁴ are both cyclopropyl, W is NCH₃, and R¹ for a compound corresponds in each case to one row of Table A.

Table 67: Compounds of the formula I.1, in which Y and X are both O, R³ and R⁴ are both cyclopropyl, W is NC₂H₅, and R¹ for a compound corresponds in each case to one row of Table A.

Table 68: Compounds of the formula I.1, in which Y and X are both O, R³ and R⁴ are both cyclopropyl, W is N—CH(═O), and R¹ for a compound corresponds in each case to one row of Table A.

Table 69: Compounds of the formula I.1, in which Y and X are both S, R³ and R⁴ are both cyclopropyl, W is NH, and R¹ for a compound corresponds in each case to one row of Table A.

Table 70: Compounds of the formula I.1, in which Y and X are both S, R³ and R⁴ are both cyclopropyl, W is NCH₃, and R¹ for a compound corresponds in each case to one row of Table A.

Table 71: Compounds of the formula I.1, in which Y and X are both S, R³ and R⁴ are both cyclopropyl, W is NC₂H₅, and R¹ for a compound corresponds in each case to one row of Table A.

Table 72: Compounds of the formula I.1, in which Y and X are both S, R³ and R⁴ are both cyclopropyl, W is N—CH(═O), and R¹ for a compound corresponds in each case to one row of Table A.

Table 73: Compounds of the formula I.1, in which Y is O and X is S, R³ and R⁴ are both cyclopropyl, W is NH, and R¹ for a compound corresponds in each case to one row of Table A.

Table 74: Compounds of the formula I.1, in which Y is O and X is S, R³ and R⁴ are both cyclopropyl, W is NCH₃, and R¹ for a compound corresponds in each case to one row of Table A.

Table 75: Compounds of the formula I.1, in which Y is O and X is S, R³ and R⁴ are both cyclopropyl, W is NC₂H₅, and R¹ for a compound corresponds in each case to one row of Table A.

Table 76: Compounds of the formula I.1, in which Y is O and X is S, R³ and R⁴ are both cyclopropyl, W is N—CH(═O), and R¹ for a compound corresponds in each case to one row of Table A.

Table 77: Compounds of the formula I.1, in which Y is S and X is O, R³ and R⁴ are both cyclopropyl, W is NH, and R¹ for a compound corresponds in each case to one row of Table A.

Table 78: Compounds of the formula I.1, in which Y is S and X is O, R³ and R⁴ are both cyclopropyl, W is NCH₃, and R¹ for a compound corresponds in each case to one row of Table A.

Table 79: Compounds of the formula I.1, in which Y is S and X is O, R³ and R⁴ are both cyclopropyl, W is NC₂H₅, and R¹ for a compound corresponds in each case to one row of Table A.

Table 80: Compounds of the formula I.1, in which Y is S and X is O, R³ and R⁴ are both cyclopropyl, W is N—CH(═O), and R¹ for a compound corresponds in each case to one row of Table A.

Table 81: Compounds of the formula I.1, in which Y and X are both O, R³ and R⁴ are both hydrogen, W is O, and R¹ for a compound corresponds in each case to one row of Table A.

Table 82: Compounds of the formula I.1, in which Y is S and X is O, R³ and R⁴ are both hydrogen, W is O, and R¹ for a compound corresponds in each case to one row of Table A.

Table 83: Compounds of the formula I.1, in which Y and X are both O, R³ and R⁴ are both F, W is O, and R¹ for a compound corresponds in each case to one row of Table A.

Table 84: Compounds of the formula I.1, in which Y is S and X is O, R³ and R⁴ are both F, W is O, and R¹ for a compound corresponds in each case to one row of Table A.

Table 85: Compounds of the formula I.1, in which Y and X are both O, R³ and R⁴ are both CH₃, W is O, and R¹ for a compound corresponds in each case to one row of Table A.

Table 86: Compounds of the formula I.1, in which Y is S and X is O, R³ and R⁴ are both CH₃, W is O, and R¹ for a compound corresponds in each case to one row of Table A.

Table 87: Compounds of the formula I.1, in which Y and X are both O, R³ is hydrogen, R⁴ is CH₃, W is O, and R¹ for a compound corresponds in each case to one row of Table A.

Table 88: Compounds of the formula I.1, in which Y is S and X is O, R³ is hydrogen, R⁴ is CH₃, W is O, and R¹ for a compound corresponds in each case to one row of Table A.

Table 89: Compounds of the formula I.1, in which Y and X are both O, R³ and R⁴ are both cyclopropyl, W is O, and R¹ for a compound corresponds in each case to one row of Table A.

Table 90: Compounds of the formula I.1, in which Y is S and X is O, R³ and R⁴ are both cyclopropyl, W is O, and R¹ for a compound corresponds in each case to one row of Table A.

TABLE A

| No. | R¹ |
|---|---|
| I-1 | H |
| I-2 | CHO |
| I-3 | CH₃ |
| I-4 | CH₂CH₃ |
| I-5 | CH₂CH₂CH₃ |
| I-6 | CH(CH₃)₂ |
| I-7 | CH₂CH₂CH₂CH₃ |
| I-8 | CH(CH₃)CH₂CH₃ |
| I-9 | CH₂CH(CH₃)CH₃ |
| I-10 | C(CH₃)₃ |
| I-11 | cyclopropyl |
| I-12 | cyclobutyl |
| I-13 | cyclopentyl |
| I-14 | cyclohexyl |
| I-15 | allyl |
| I-16 | propargyl |
| I-17 | —C(═O)NH₂ |
| I-18 | —C(═O)NH(CH₃) |
| I-19 | —C(═O)NH(CH₂CH₃) |
| I-20 | —C(═O)NH(CH₂CH₂CH₃) |
| I-21 | —C(═O)NH(CH(CH₃)₂) |
| I-22 | CH₂C(═O)NH₂ |
| I-23 | CH₂C(═O)NH(CH₃) |
| I-24 | CH₂C(═O)NH(CH₂CH₃) |
| I-25 | CH₂C(═O)NH(CH₂CH₂CH₃) |
| I-26 | CH₂C(═O)NH(CH(CH₃)₂) |
| I-27 | OCH₃ |
| I-28 | OCH₂CH₃ |
| I-29 | OCH₂CH₂CH₃ |
| I-30 | OCH(CH₃)₂ |
| I-31 | OCH₂CH₂CH₂CH₃ |
| I-32 | OCH(CH₃)CH₂CH₃ |
| I-33 | OCH₂CH(CH₃)CH₃ |
| I-34 | CF₃ |
| I-35 | CH₂CF₃ |
| I-36 | CF₂CF₃ |
| I-37 | CHF₂ |
| I-38 | OCHF₂ |
| I-39 | CN |
| I-40 | phenyl |
| I-41 | 2-F-phenyl |
| I-42 | 4-F-phenyl |
| I-43 | 2,4-F₂-phenyl |
| I-44 | 2-Cl-phenyl |
| I-45 | 4-Cl-phenyl |
| I-46 | 2,4-Cl₂-phenyl |
| I-47 | benzyl |
| I-48 | 2-F-benzyl |
| I-49 | 4-F-benzyl |
| I-50 | 2,4-F₂-benzyl |
| I-51 | 2-Cl-benzyl |
| I-52 | 4-Cl-benzyl |
| I-53 | 2,4-Cl₂-benzyl |
| I-54 | bicyclo[1.1.1]pentan-1-yl |

The compounds I can be prepared according to methods or in analogy to methods that are described in the prior art. The synthesis takes advantage of starting materials that are commercially available or may be prepared according to conventional procedures starting from readily available compounds.

For example, compounds I can be prepared from ester of formula II via treatment with compounds of formula VIII in a trimethylaluminium (TMAL) or lanthanum trifluoromethanesulfonate mediated direct amide bond formation as described in Org. Synth. 59, 49, 1979; Org. Lett. 16(7), 2018-2021, 2014 and Chem. Commun. (Cambridge) (9), 1100-1102, 2008 in a suitable solvent (e.g. toluene or dichloromethane) at a temperature between 20° C. and 140° C.

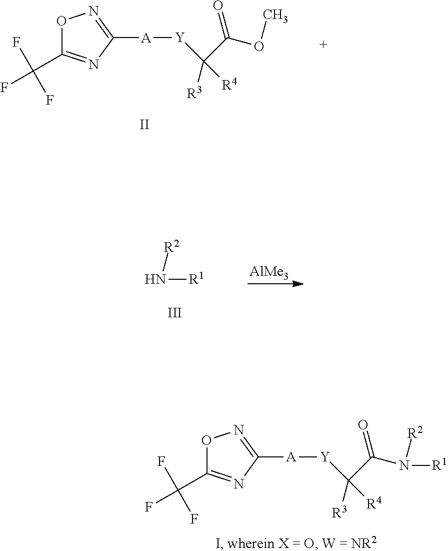

Compounds I, wherein X is O can be converted to compounds I, wherein X is S, by reacting with an appropriate sulfonation reagent, preferably Lawessons reagent, in an organic solvent, preferably at temperatures between 20° C. and 150° C., preferably at 110° C., as previously described (see for example Journal of Org. Chem. 73, 9102, 2008; or Eur. J. Org. Chem. 30, 6687, 2015).

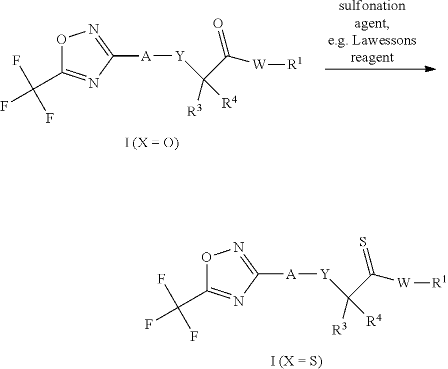

Compounds II can be prepared by reacting amidoximes of formula IV with trifluoroacetic anhydride (TFAA) in an organic solvent, e.g. dichloromethane or THF, at temperatures between 0° C. and 100° C., preferably at about 25° C., as previously described in WO 2013/008162.

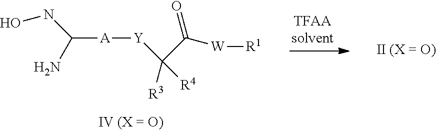

A skilled person will recognize that compounds IV can be accessed by treating nitriles V with hydroxylamine (or its HCl salt) in an organic solvent and in the presence of a base (for precedents see for example WO2009/074950, WO2006/013104, EP1932843).

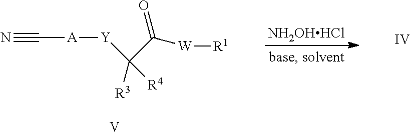

Compounds V can be accessed from the respective thio or hydroxy compounds VI with methyl 2-bromo acetate VII in an organic solvent and in the presence of a base (for precedents see for example Org. Lett. 18(18), 4570-4573, 2016; Bioorq. & Med. Chem. 23(1), 132-140, 2015; and WO 2016/011019). Preferably, an polar aprotic solvent (e.g. dimethylformamide (DMF), THF, acetonitrile) and an inorganic base are used, more preferably DMF and potassium or cesium carbonate. The reaction is best performed at elevated temperatures, most preferably in the range between 60° C. and 120° C.

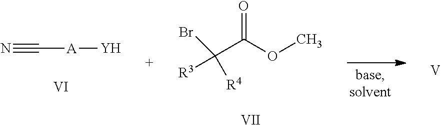

Another embodiment of the invention relates to intermediate compounds of the formulae IV or V, wherein the variables A, Y, W, R¹, R³ and R⁴ are as defined or preferably defined herein for compounds of the formula I; preferably A is phenyl.

In a preferred embodiment the invention relates to intermediate compounds of the formulae IV.a or V.a,

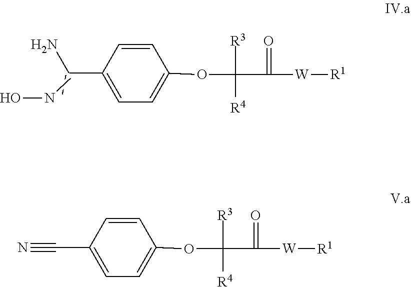

wherein the variables W, R¹, R³ and R⁴ are as defined or preferably defined herein for compounds of the formula I.

In a further embodiment the invention relates to intermediate compounds of the formulae IV.a or V.a, wherein R³ and R⁴ are both fluorine; and R¹, W are as defined herein for compounds of the formula I.

In a further embodiment the invention relates to intermediate compounds of the formulae IV.a or V.a, wherein R³ and R⁴ are both fluorine; W is O; R¹ is hydrogen or C₁-C₆-alkyl, in particular hydrogen, methyl or ethyl.

Especially preferred intermediates are compounds of the formulae IV.a or V.a, wherein R³ and R⁴ are both fluorine; W is NR²; and R² independently of each other are selected from the group consisting of hydrogen, methyl, ethyl, n-propyl, n-butyl, 1-methylpropyl, 2-methylpropyl, tert-butyl, cyclopropyl, cyclopropyl-CH₂—, allyl or propargyl; and wherein R¹ is as defined or preferably defined herein for compounds of the formula I; particularly R¹ is hydrogen, C₁-C₆-alkyl, C₃-C₈-cycloalkyl, C₃-C₈-cycloalkenyl, C₂-C₆-alkenyl, C₂-C₆-alkynyl or phenyl; and wherein any of the aliphatic or cyclic groups in R¹ are unsubstituted or substituted with 1, 2, 3, 4 or up to the maximum possible number of identical or different radicals selected from the group consisting of halogen; more particularly R¹ is hydrogen, methyl, ethyl, n-propyl, iso-propyl, n-butyl, 1-methylpropyl, 2-methylpropyl, tert-butyl, cyclopropyl, cyclopropyl-CH₂—, allyl or propargyl.

The compounds I and the compositions according to the invention are particularly important in the control of a multitude of phytopathogenic fungi on various cultivated plants, such as cereals, e.g. wheat, rye, barley, triticale, oats or rice; beet, e.g. sugar beet or fodder beet; fruits, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp orjute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e.g. conifers; and on the plant propagation material, such as seeds, and the crop material of these plants.

Preferably, compounds I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by breeding, mutagenesis or genetic engineering including but not limiting to agricultural biotech products on the market or in development (cf. http://cera-gmc.org/, see GM crop database therein). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e.g. by glycosylation or polymer additions such as prenylated, acetylated or farnesylated moieties or PEG moieties.

Mutagenesis includes techniques of random mutagenesis using X-rays or mutagenic chemicals, but also techniques of targeted mutagenesis, to create mutations at a specific locus of a plant genome. Targeted mutagenesis techniques frequently use oligonucleotides or proteins like CRISPR/Cas, zinc-finger nucleases, TALENs or meganucleases to achieve the targeting effect. Genetic engineering usually uses recombinant DNA techniques to create modifications in a plant genome which under natural circumstances cannot readily be obtained by cross breeding, mutagenesis or natural recombination. Typically, one or more genes are integrated into the genome of a plant to add a trait or improve a trait. These integrated genes are also referred to as transgenes in the art, while plant comprising such transgenes are referred to as transgenic plants. The process of plant transformation usually produces several transformation events, which differ in the genomic locus in which a transgene has been integrated. Plants comprising a specific transgene on a specific genomic locus are usually described as comprising a specific “event”, which is referred to by a specific event name. Traits which have been introduced in plants or have been modified include herbicide tolerance, insect resistance, increased yield and tolerance to abiotic conditions, like drought.

Herbicide tolerance has been created by using mutagenesis as well as using genetic engineering. Plants which have been rendered tolerant to acetolactate synthase (ALS) inhibitor herbicides by mutagenesis and breeding comprise plant varieties commercially available under the name Clearfield®.

Herbicide tolerance has been created via the use of transgenes to glyphosate, glufosinate, 2,4-D, dicamba, oxynil herbicides, like bromoxynil and ioxynil, sulfonylurea herbicides, ALS inhibitors and 4-hydroxyphenylpyruvate dioxygenase (HPPD) inhibitors, like isoxaflutole and mesotrione. Transgenes which have been used to provide herbicide tolerance traits comprise: for tolerance to glyphosate: cp4 epsps, epsps grg23ace5, mepsps, 2mepsps, gat4601, gat4621, goxv247; for tolerance to glufosinate: pat and bar, for tolerance to 2,4-D: aad-1, aad-12; for tolerance to dicamba: dmo; for tolerance to oxynil herbicies: bxn; for tolerance to sulfonylurea herbicides: zm-hra, csrl-2, gm-hra, S4-HrA; for tolerance to ALS inhibitors: csrl-2; and for tolerance to HPPD inhibitors: hppdPF, W336, avhppd-03.

Transgenic corn events comprising herbicide tolerance genes include, but are not limited to, DAS40278, MON801, MON802, MON809, MON810, MON832, MON87411, MON87419, MON87427, MON88017, MON89034, NK603, GA21, MZHG0JG, HCEM485, VCO-ø1981-5, 676, 678, 680, 33121, 4114, 59122, 98140, Bt10, Bt176, CBH-351, DBT418, DLL25, MS3, MS6, MZIR098, T25, TC1507 and TC6275.

Transgenic soybean events comprising herbicide tolerance genes include, but are not limited to, GTS 40-3-2, MON87705, MON87708, MON87712, MON87769, MON89788, A2704-12, A2704-21, A5547-127, A5547-35, DP356043, DAS44406-6, DAS68416-4, DAS-81419-2, GU262, SYHTøH2, W62, W98, FG72 and CV127.

Transgenic cotton events comprising herbicide tolerance genes include, but are not limited to, 19-51a, 31707, 42317, 81910, 281-24-236, 3006-210-23, BXN10211, BXN10215, BXN10222, BXN10224, MON1445, MON1698, MON88701, MON88913, GHB119, GHB614, LLCotton25, T303-3 and T304-40.

Transgenic canola events comprising herbicide tolerance genes are for example, but not excluding others, MON88302, HCR-1, HCN10, HCN28, HCN92, MS1, MS8, PHY14, PHY23, PHY35, PHY36, RF1, RF2 and RF3.

Insect resistance has mainly been created by transferring bacterial genes for insecticidal proteins to plants: Transgenes which have most frequently been used are toxin genes of Bacillus spp. and synthetic variants thereof, like cry1A, cry1Ab, cry1Ab-Ac, cry1Ac, cry1A.105, cry1F, cry1Fa2, cry2Ab2, cry2Ae, mcry3A, ecry3.1Ab, cry3Bb1, cry34Ab1, cry35Ab1, cry9C, vip3A(a), vip3Aa20. However, also genes of plant origin, such as genes coding for protease inhibitors, like CpTI and pinII, have been transferred to other plants. A further approach uses transgenes such as dvsnf7 to produce double-stranded RNA in plants.

Transgenic corn events comprising genes for insecticidal proteins or double stranded RNA include, but are not limited to, Bt10, Bt11, Bt176, MON801, MON802, MON809, MON810, MON863, MON87411, MON88017, MON89034, 33121, 4114, 5307, 59122, TC1507, TC6275, CBH-351, MIR162, DBT418 and MZIR098. Transgenic soybean events comprising genes for insecticidal proteins include, but are not limited to, MON87701, MON87751 and DAS-81419. Transgenic cotton events comprising genes for insecticidal proteins include, but are not limited to, SGK321, MON531, MON757, MON1076, MON15985, 31707, 31803, 31807, 31808, 42317, BNLA-601, Event1, COT67B, COT102, T303-3, T304-40, GFM Cry1A, GK12, MLS 9124, 281-24-236, 3006-210-23, GHB119 and SGK321.

Increased yield has been created by using the transgene athb17, being present for example in corn event MON87403, or by using the transgene bbx32, being present for example in the soybean event MON87712.

Cultivated plants comprising a modified oil content have been created by using the transgenes: gm-fad2-1, Pj.D6D, Nc.Fad3, fad2-1A and fatb1-A. Soybean events comprising at least one of these genes are: 260-05, MON87705 and MON87769.

Tolerance to abiotic conditions, such as drought, has been created by using the transgene cspB, comprised by the corn event MON87460 and by using the transgene Hahb-4, comprised by soybean event IND-øø41ø-5.

Traits are frequently combined by combining genes in a transformation event or by combining different events during the breeding process resulting in a cultivated plant with stacked traits. Preferred combinations of traits are combinations of herbicide tolerance traits to different groups of herbicides, combinations of insect tolerance to different kind of insects, in particular tolerance to lepidopteran and coleopteran insects, combinations of herbicide tolerance with one or several types of insect resistance, combinations of herbicide tolerance with increased yield as well as combinations of herbicide tolerance and tolerance to abiotic conditions.

Plants comprising singular or stacked traits as well as the genes and events providing these traits are well known in the art. For example, detailed information as to the mutagenized or integrated genes and the respective events are available from websites of the organizations “International Service for the Acquisition of Agri-biotech Applications (ISAAA)” (http://www.isaaa.org/gmapprovaldatabase) and the “Center for Environmental Risk Assessment (CERA)” (http://cera-gmc.org/GMCropDatabase). Further information on specific events and methods to detect them can be found for canola events MS1, MS8, RF3, GT73, MON88302, KK179 in WO01/031042, WO01/041558, WO01/041558, WO002/036831, WO11/153186, WO13/003558, for cotton events MON1445, MON15985, MON531 (MON15985), LLCotton25, MON88913, COT102, 281-24-236, 3006-210-23, COT67B, GHB614, T304-40, GHB119, MON88701, 81910 in WO02/034946, WO02/100163, WO02/100163, WO03/013224, WO04/072235, WO04/039986, WO05/103266, WO05/103266, WO06/128573, WO07/017186, WO08/122406, WO08/151780, WO12/134808, WO13/112527; for corn events GA21, MON810, DLL25, TC1507, MON863, MIR604, LY038, MON88017, 3272, 59122, NK603, MIR162, MON89034, 98140, 32138, MON87460, 5307, 4114, MON87427, DAS40278, MON87411, 33121, MON87403, MON87419 in WO98/044140, U.S. Ser. No. 02/102,582, U.S. Ser. No. 03/126,634, WO04/099447, WO04/011601, WO05/103301, WO05/061720, WO05/059103, WO06/098952, WO06/039376, US2007/292854, WO07/142840, WO07/140256, WO08/112019, WO09/103049, WO09/111263, WO10/077816, WO11/084621, WO11/062904, WO11/022469, WO13/169923, WO14/116854, WO15/053998, WO15/142571; for potato events E12, F10, J3, J55, V11, X17, Y9 in WO14/178910, WO14/178913, WO14/178941, WO14/179276, WO16/183445, WO17/062831, WO17/062825; for rice events LLRICE06, LLRICE601, LLRICE62 in WO00/026345, WO0/026356, WO0/026345; and for soybean events H7-1, MON89788, A2704-12, A5547-127, DP305423, DP356043, MON87701, MON87769, CV127, MON87705, DAS68416-4, MON87708, MON87712, SYHT0H2, DAS81419, DAS81419×DAS44406-6, MON87751 in WO04/074492, WO06/130436, WO06/108674, WO06/108675, WO08/054747, WO08/002872, WO09/064652, WO09/102873, WO10/080829, WO10/037016, WO11/066384, WO11/034704, WO12/051199, WO12/082548, WO13/016527, WO13/016516, WO14/201235.

The use of compounds I and compositions according to the invention, respectively, on cultivated plants may result in effects which are specific to a cultivated plant comprising a certain gene or event. These effects might involve changes in growth behavior or changed resistance to biotic or abiotic stress factors. Such effects may in particular comprise enhanced yield, enhanced resistance or tolerance to insects, nematodes, fungal, bacterial, mycoplasma, viral or viroid pathogens as well as early vigour, early or delayed ripening, cold or heat tolerance as well as changed amino acid or fatty acid spectrum or content.

The compounds I and compositions thereof, respectively, are particularly suitable for controlling the following plant diseases:

Albugo spp. (white rust) on ornamentals, vegetables (e.g. A. candida) and sunflowers (e.g. A. tragopogonis); Alternaria spp. (Alternaria leaf spot) on vegetables, rape (A. brassicola or brassicae), sugar beets (A. tenuis), fruits, rice, soybeans, potatoes (e.g. A. solani or A. alternata), tomatoes (e.g. A. solani or A. alternata) and wheat; Aphanomyces spp. on sugar beets and vegetables; Ascochyta spp. on cereals and vegetables, e.g. A. tritici (anthracnose) on wheat and A. hordei on barley; Bipolaris and Drechslera spp. (teleomorph: Cochliobolus spp.), e.g. Southern leaf blight (D. maydcis) or Northern leaf blight (B. zeicola) on corn, e.g. spot blotch (B. sorokiniana) on cereals and e.g. B. oryzae on rice and turfs; Blumeria (formerly Erysiphe) graminis (powdery mildew) on cereals (e.g. on wheat or barley); Botrytis cinerea (teleomorph: Botryotinia fuckeliana: grey mold) on fruits and berries (e.g. strawberries), vegetables (e.g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e.g. C. ulmi (Dutch elm disease) on elms; Cercospora spp. (Cercospora leaf spots) on corn (e.g. Gray leaf spot: C. zeae-maydis), rice, sugar beets (e.g. C. beticola), sugar cane, vegetables, coffee, soybeans (e.g. C. sojina or C. kikuchii) and rice; Cladosporium spp. on tomatoes (e.g. C. fulvum: leaf mold) and cereals, e.g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e.g. C. sativus, anamorph: B. sorokiniana) and rice (e.g. C. miyabeanus, anamorph: H. oryzae); Colletotrichum (teleomorph: Glomerella) spp. (anthracnose) on cotton (e.g. C. gossypii), corn (e.g. C. graminicola: Anthracnose stalk rot), soft fruits, potatoes (e.g. C. coccodes black dot), beans (e.g. C. lindemuthianum) and soybeans (e.g. C. truncatum or C. gloeosporioides); Corticium spp., e.g. C. sasakii (sheath blight) on rice; Corynespora cassiicola (leaf spots) on soybeans and ornamentals; Cycloconium spp., e.g. C. oleaginum on olive trees; Cylindrocarpon spp. (e.g. fruit tree canker or young vine decline, teleomorph: Nectria or Neonectria spp.) on fruit trees, vines (e.g. C. liriodendri, teleomorph: Neonectria liriodendri Black Foot Disease) and ornamentals; Dematophora (teleomorph: Rosellinia) necatrix (root and stem rot) on soybeans; Diaporthe spp., e.g. D. phaseolorum (damping off) on soybeans; Drechslera (syn. Helminthosporium, teleomorph: Pyrenophora) spp. on corn, cereals, such as barley (e.g. D. teres, net blotch) and wheat (e.g. D. tritici-repentis. tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e.g. E. pisi), such as cucurbits (e.g. E. cichoracearum), cabbages, rape (e.g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e.g. E. turcicum); Fusarium (teleomorph: Gibberella) spp. (wilt, root or stem rot) on various plants, such as F. graminearum or F. culmorum (root rot, scab or head blight) on cereals (e.g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e.g. wheat or barley) and corn; Gibberella spp. on cereals (e.g. G. zeae) and rice (e.g. G. fujikuroi Bakanae disease); Glomerella cingulata on vines, pome fruits and other plants and G. gossypii on cotton; Grain staining complex on rice; Guignardia bidwellii (black rot) on vines; Gymnosporangium spp. on rosaceous plants and junipers, e.g. G. sabinae (rust) on pears; Helminthosporium spp. (syn. Drechslera, teleomorph: Cochliobolus) on corn, cereals and rice; Hemileia spp., e.g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e.g. wheat or barley); Microsphaera diffusa (powdery mildew) on soybeans; Monilinia spp., e.g. M. laxa, M. fructicola and M. fructigena (bloom and twig blight, brown rot) on stone fruits and other rosaceous plants; Mycosphaerella spp. on cereals, bananas, soft fruits and ground nuts, such as e.g. M. graminicola (anamorph: Septoria tritici, Septoria blotch) on wheat or M. fijiensis (black Sigatoka disease) on bananas; Peronospora spp. (downy mildew) on cabbage (e.g. P. brassicae), rape (e.g. P. parasitica), onions (e.g. P. destructor), tobacco (P. tabacina) and soybeans (e.g. P. manshurica); Phakopsora pachyrhizi and P. meibomiae (soybean rust) on soybeans; Phialophora spp. e.g. on vines (e.g. P. tracheiphila and P. tetraspora) and soybeans (e.g. P. gregata: stem rot); Phoma lingam (root and stem rot) on rape and cabbage and P. betae (root rot, leaf spot and damping-off) on sugar beets; Phomopsis spp. on sunflowers, vines (e.g. P. viticola: can and leaf spot) and soybeans (e.g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e.g. P. capsici), soybeans (e.g. P. megasperma, syn. P. sojae), potatoes and tomatoes (e.g. P. infestans late blight) and broad-leaved trees (e.g. P. ramorum, sudden oak death); Plasmodiophora brassicae (club root) on cabbage, rape, radish and other plants; Plasmopara spp., e.g. P. viticola (grapevine downy mildew) on vines and P. halstedii on sunflowers; Podosphaera spp. (powdery mildew) on rosaceous plants, hop, pome and soft fruits, e.g. P. leucotricha on apples; Polymyxa spp., e.g. on cereals, such as barley and wheat (P. graminis) and sugar beets (P. betae) and thereby transmitted viral diseases; Pseudocercosporella herpotrichoides (eyespot, teleomorph: Tapesia yaundae) on cereals, e.g. wheat or barley; Pseudoperonospora (downy mildew) on various plants, e.g. P. cubensison cucurbits or P. humili on hop; Pseudopezicula tracheiphila (red fire disease or ‘rotbrenner’, anamorph: Phialophora) on vines; Puccinia spp. (rusts) on various plants, e.g. P. triticina (brown or leaf rust), P. striiformis (stripe or yellow rust), P. hordei (dwarf rust), P. graminis (stem or black rust) or P. recondita (brown or leaf rust) on cereals, such as e.g. wheat, barley or rye, P. kuehnii/(orange rust) on sugar cane and P. asparagi on asparagus; Pyrenophora (anamorph: Drechslera) tritici-repentis (tan spot) on wheat or P. teres (net blotch) on barley; Pyricularia spp., e.g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e.g. P. ultimum or P. aphanidermatum); Ramularia spp., e.g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e.g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e.g. S. sclerotiorum) and soybeans (e.g. S. rolfsii or S. sclerotiorum); Septoria spp. on various plants, e.g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e.g. S. turcicum, syn. Helminthosporium turcicum) and turf; Sphacelotheca spp. (smut) on corn, (e.g. S. reiliana: head smut), sorghum und sugar cane; Sphaerotheca fuiginea (powdery mildew) on cucurbits; Spongospora subterranea (powdery scab) on potatoes and thereby transmitted viral diseases; Stagonospora spp. on cereals, e.g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e.g. T. deformans (leaf curl disease) on peaches and T. pruni (plum pocket) on plums; Thielaviopsis spp. (black root rot) on tobacco, pome fruits, vegetables, soybeans and cotton, e.g. T. basicola (syn. Chalara elegans); Tilletia spp. (common bunt or stinking smut) on cereals, such as e.g. T. tritici (syn. T. caries, wheat bunt) and T. controversa (dwarf bunt) on wheat; Typhula incarnata (grey snow mold) on barley or wheat; Uro-cystis spp., e.g. U. occulta (stem smut) on rye; Uromyces spp. (rust) on vegetables, such as beans (e.g. U. appendiculatus, syn. U. phaseoli) and sugar beets (e.g. U. betae); Ustilago spp. (loose smut) on cereals (e.g. U. nuda and U. avaenae), corn (e.g. U. maydis corn smut) and sugar cane; Venturia spp. (scab) on apples (e.g. V. inaequalis) and pears; and Verticillium spp. (wilt) on various plants, such as fruits and ornamentals, vines, soft fruits, vegetables and field crops, e.g. V. dahliae on strawberries, rape, potatoes and tomatoes.

In a preferred embodiment the compounds I, their mixtures with other active compounds as defined herein and compositions thereof, respectively, are particularly suitable for controlling the following plant diseases: Puccinia spp. (rusts) on various plants, for example, but not limited to P. triticina (brown or leaf rust), P. striiformis (stripe or yellow rust), P. hordei (dwarf rust), P. graminis (stem or black rust) or P. recondita (brown or leaf rust) on cereals, such as e.g. wheat, barley or rye, and Puccinia sorghi (common rust) on maize, Puccinia polysora (southern rust) on maize; and Phakopsoraceae spp. on various plants, in particular Phakopsora pachyrhizi and P. meibomiae (soybean rust) on soybeans.

The compounds I and compositions thereof, respectively, are also suitable for controlling harmful fungi in the protection of stored products or harvest and in the protection of materials.

The term “protection of materials” is to be understood to denote the protection of technical and non-living materials, such as adhesives, glues, wood, paper and paperboard, textiles, leather, paint dispersions, plastics, cooling lubricants, fiber or fabrics, against the infestation and destruction by harmful microorganisms, such as fungi and bacteria. As to the protection of wood and other materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Conlophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of stored products and harvest the following yeast fungi are worthy of note: Candida spp. and Saccharomyces cerevisae.

The method of treatment according to the invention can also be used in the field of protecting stored products or harvest against attack of fungi and microorganisms. According to the present invention, the term “stored products” is understood to denote natural substances of plant or animal origin and their processed forms, which have been taken from the natural life cycle and for which long-term protection is desired. Stored products of crop plant origin, such as plants or parts thereof, for example stalks, leafs, tubers, seeds, fruits or grains, can be protected in the freshly harvested state or in processed form, such as pre-dried, moistened, comminuted, ground, pressed or roasted, which process is also known as post-harvest treatment. Also falling under the definition of stored products is timber, whether in the form of crude timber, such as construction timber, electricity pylons and barriers, or in the form of finished articles, such as furniture or objects made from wood. Stored products of animal origin are hides, leather, furs, hairs and the like. The combinations according the present invention can prevent disadvantageous effects such as decay, discoloration or mold. Preferably “stored products” is understood to denote natural substances of plant origin and their processed forms, more preferably fruits and their processed forms, such as pomes, stone fruits, soft fruits and citrus fruits and their processed forms.

The compounds I and compositions thereof, respectively, may be used for improving the health of a plant. The invention also relates to a method for improving plant health by treating a plant, its propagation material and/or the locus where the plant is growing or is to grow with an effective amount of compounds I and compositions thereof, respectively.

The term “plant health” is to be understood to denote a condition of the plant and/or its products which is determined by several indicators alone or in combination with each other such as yield (e.g. increased biomass and/or increased content of valuable ingredients), plant vigor (e.g. improved plant growth and/or greener leaves (“greening effect”)), quality (e.g. improved content or composition of certain ingredients) and tolerance to abiotic and/or biotic stress. The above identified indicators for the health condition of a plant may be interdependent or may result from each other.

The compounds of formula I can be present in different crystal modifications whose biological activity may differ. They are likewise subject matter of the present invention.

The compounds I are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compounds I as such or a composition comprising at least one compound I prophylactically either at or before planting or transplanting.

The invention also relates to agrochemical compositions comprising an auxiliary and at least one compound I according to the invention.

An agrochemical composition comprises a fungicidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, their N-oxides and salts can be converted into customary types of agrochemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6^{th} Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e.g. toluene, paraffin, tetrahydronaphthalene, alkylated naphthalenes; alcohols, e.g. ethanol, propanol, butanol, benzyl alcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methyl pyrrolidone, fatty acid dimethyl amides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl naphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide.

Suitable polyelectrolytes are polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethyl cellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinyl pyrrolidones, polyvinyl acetates, polyvinyl alcohols, polyacrylates, biological or synthetic waxes, and cellulose ethers.

Examples for composition types and their preparation are:

i) Water-soluble concentrates (SL, LS)

10-60 wt % of a compound I and 5-15 wt % wetting agent (e.g. alcohol alkoxylates) are dissolved in water and/or in a water-soluble solvent (e.g. alcohols) ad 100 wt %. The active substance dissolves upon dilution with water.

ii) Dispersible concentrates (DC)

5-25 wt % of a compound I and 1-10 wt % dispersant (e.g. polyvinyl pyrrolidone) are dissolved in organic solvent (e.g. cyclohexanone) ad 100 wt %. Dilution with water gives a dispersion.

iii) Emulsifiable concentrates (EC)

15-70 wt % of a compound I and 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.

iv) Emulsions (EW, EO, ES)

5-40 wt % of a compound I and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into water ad 100 wt % by means of an emulsifying machine and made into a homogeneous emulsion. Dilution with water gives an emulsion.

v) Suspensions (SC, OD, FS)

In an agitated ball mill, 20-60 wt % of a compound I are comminuted with addition of 2-10 wt % dispersants and wetting agents (e.g. sodium lignosulfonate and alcohol ethoxylate), 0.1-2 wt % thickener (e.g. xanthan gum) and water ad 100 wt % to give a fine active substance suspension. Dilution with water gives a stable suspension of the active substance. For FS type composition up to 40 wt % binder (e.g. polyvinyl alcohol) is added.

vi) Water-dispersible granules and water-soluble granules (WG, SG)

50-80 wt % of a compound I are ground finely with addition of dispersants and wetting agents (e.g. sodium lignosulfonate and alcohol ethoxylate) ad 100 wt % and prepared as water-dispersible or water-soluble granules by means of technical appliances (e.g. extrusion, spray tower, fluidized bed). Dilution with water gives a stable dispersion or solution of the active substance.

vii) Water-dispersible powders and water-soluble powders (WP, SP, WS)

50-80 wt % of a compound I are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.

viii) Gel (GW, GF)

In an agitated ball mill, 5-25 wt % of a compound I are comminuted with addition of 3-10 wt % dispersants (e.g. sodium lignosulfonate), 1-5 wt % thickener (e.g. carboxymethyl cellulose) and water ad 100 wt % to give a fine suspension of the active substance. Dilution with water gives a stable suspension of the active substance.

ix) Microemulsion (ME)

5-20 wt % of a compound I are added to 5-30 wt % organic solvent blend (e.g. fatty acid dimethyl amide and cyclohexanone), 10-25 wt % surfactant blend (e.g. alcohol ethoxylate and arylphenol ethoxylate), and water ad 100%. This mixture is stirred for 1 h to produce spontaneously a thermodynamically stable microemulsion.

x) Microcapsules (CS)

An oil phase comprising 5-50 wt % of a compound I, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization results in the formation of poly(meth)acrylate microcapsules. Alternatively, an oil phase comprising 5-50 wt % of a compound I according to the invention, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexamethylenediamine) results in the formation of polyurea microcapsules. The monomers amount to 1-10 wt %. The wt % relate to the total CS composition.

xi) Dustable powders (DP, DS)

1-10 wt % of a compound I are ground finely and mixed intimately with solid carrier (e.g. finely divided kaolin) ad 100 wt %.

xii) Granules (GR, FG)

0.5-30 wt % of a compound I is ground finely and associated with solid carrier (e.g. silicate) ad 100 wt %. Granulation is achieved by extrusion, spray-drying or fluidized bed.

xiii) Ultra-low volume liquids (UL)

1-50 wt % of a compound I are dissolved in organic solvent (e.g. aromatic hydrocarbon) ad 100 wt %.

The compositions types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, more preferably between 1 and 70%, and in particular between 10 and 60%, by weight of active substance. The active substances are employed in a purity of from 90% to 100%, preferably from 95% to 100% (according to NMR spectrum).

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed. The compositions in question give, after two-to-tenfold dilution, active substance concentrations of from 0.01 to 60% by weight, preferably from 0.1 to 40%, in the ready-to-use preparations. Application can be carried out before or during sowing. Methods for applying compound I and compositions thereof, respectively, onto plant propagation material, especially seeds, include dressing, coating, pelleting, dusting, and soaking as well as in-furrow application methods. Preferably, compound I or the compositions thereof, respectively, are applied on to the plant propagation material by a method such that germination is not induced, e.g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e.g. herbicides, insecticides, fungicides, growth regulators, safeners, biopesticides) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

A pesticide is generally a chemical or biological agent (such as pestidal active ingredient, compound, composition, virus, bacterium, antimicrobial or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease. The term “pesticide” includes also plant growth regulators that alter the expected growth, flowering, or reproduction rate of plants; defoliants that cause leaves or other foliage to drop from a plant, usually to facilitate harvest; desiccants that promote drying of living tissues, such as unwanted plant tops; plant activators that activate plant physiology for defense of against certain pests; safeners that reduce unwanted herbicidal action of pesticides on crop plants; and plant growth promoters that affect plant physiology e.g. to increase plant growth, biomass, yield or any other quality parameter of the harvestable goods of a crop plant.

Biopesticides have been defined as a form of pesticides based on microorganisms (bacteria, fungi, viruses, nematodes, etc.) or natural products (compounds, such as metabolites, proteins, or extracts from biological or other natural sources) (U.S. Environmental Protection Agency: http://www.epa.gov/pesticides/biopesticides/). Biopesticides fall into two major classes, microbial and biochemical pesticides:
(1) Microbial pesticides consist of bacteria, fungi or viruses (and often include the metabolites that bacteria and fungi produce). Entomopathogenic nematodes are also classified as microbial pesticides, even though they are multi-cellular.
(2) Biochemical pesticides are naturally occurring substances that control pests or provide other crop protection uses as defined below, but are relatively non-toxic to mammals.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank or any other kind of vessel used for applications (e.g. seed treater drums, seed pelleting machinery, knapsack sprayer) and further auxiliaries may be added, if appropriate. When living microorganisms, such as microbial pesticides from groups L1), L3) and L5), form part of such kit, it must be taken care that choice and amounts of the components (e.g. chemical pesticides) and of the further auxiliaries should not influence the viability of the microbial pesticides in the composition mixed by the user. Especially for bactericides and solvents, compatibility with the respective microbial pesticide has to be taken into account.

Consequently, one embodiment of the invention is a kit for preparing a usable pesticidal composition, the kit comprising a) a composition comprising component 1) as defined herein and at least one auxiliary; and b) a composition comprising component 2) as defined herein and at least one auxiliary; and optionally c) a composition comprising at least one auxiliary and optionally a further active component 3) as defined herein.

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of pesticides II (e.g. pesticidally-active substances and biopesticides), in conjunction with which the compounds I can be used, is intended to illustrate the possible combinations but does not limit them:
A) Respiration Inhibitors
   Inhibitors of complex III at Qₒ site: azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxystrobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylideneaminooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21a), methyl-N-[2-[(1,4-dimethyl-5-phenyl-pyrazol-3-yl)oxylmethyl]phenyl]-N-methoxy-carbamate (A.1.22), metyltetrapole (A.1.25), (Z,2E)-5-[1-(2,4-dichlorophenyl)pyrazol-3-yl]-oxy-2-methoxyimino-N,3-dimethyl-pent-3-enamide (A.1.34), (Z,2E)-5-[1-(4-chlorophenyl)pyrazol-3-yl]oxy-2-methoxyimino-N,3-dimethyl-pent-3-enamide (A.1.35), pyriminostrobin (A.1.36), bifujunzhi (A.1.37), 2-(ortho-((2,5-dimethylphenyl-oxymethylen)phenyl)-3-methoxy-acrylic acid methylester (A.1.38);
   inhibitors of complex III at Qᵢ site: cyazofamid (A.2.1), amisulbrom (A.2.2), [(6S,7R,8R)-8-benzyl-3-[(3-hydroxy-4-methoxy-pyridine-2-carbonyl)amino]-6-methyl-4,9-di-oxo-1,5-dioxonan-7-yl] 2-methylpropanoate (A.2.3), fenpicoxamid (A.2.4), florylpicoxamid (A.2.5);
   inhibitors of complex II: benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.15), penthiopyrad (A.3.16), pydiflumetofen (A.3.17), pyraziflumid (A.3.18), sedaxane (A.3.19), tecloftalam (A.3.20), thifluzamide (A.3.21), inpyrfluxam (A.3.22), pyrapropoyne (A.3.23), fluindapyr (A.3.28), methyl (E)-2-[2-[(5-cyano-2-methyl-phenoxy)methyl] phenyl]-3-methoxy-prop-2-enoate (A.3.30), isoflucypram (A.3.31), 2-(difluoromethyl)-N-(1,1,3-trimethyl-indan-4-yl)pyridine-3-carboxamide (A.3.32), 2-(difluoromethyl)-N-[(3R)-1,1,3-trimethylindan-4-yl]pyridine-3-carboxamide (A.3.33), 2-(difluoromethyl)-N-(3-ethyl-1,1-dimethyl-indan-4-yl)pyridine-3-carboxamide (A.3.34), 2-(difluoromethyl)-N-[(3R)-3-ethyl-1,1-dimethyl-indan-4-yl]pyridine-3-carboxamide (A.3.35), 2-(difluoromethyl)-N-(1,1-dimethyl-3-propyl-indan-4-yl)pyridine-3-carboxamide (A.3.36), 2-(difluoromethyl)-N-[(3R)-1,1-dimethyl-3-propyl-indan-4-yl] pyridine-3-carboxamide (A.3.37), 2-(difluoromethyl)-N-(3-isobutyl-1,1-dimethyl-indan-4-yl)pyridine-3-carboxamide (A.3.38), 2-(difluoromethyl)-N-[(3R)-3-isobutyl-1,1-dimethyl-indan-4-yl] pyridine-3-carboxamide (A.3.39);
   other respiration inhibitors: diflumetorim (A.4.1); nitrophenyl derivates: binapacryl (A.4.2), dinobuton (A.4.3), dinocap (A.4.4), fluazinam (A.4.5), meptyldinocap (A.4.6), ferimzone (A.4.7); organometal compounds: fentin salts, e.g. fentin-acetate (A.4.8), fentin chloride (A.4.9) or fentin hydroxide (A.4.10); ametoctradin (A.4.11); silthiofam (A.4.12);
B) Sterol Biosynthesis Inhibitors (SBI Fungicides)
   C14 demethylase inhibitors: triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 2-(2,4-difluorophenyl)-1,1-difluoro-3-(tetrazol-1-yl)-1-[5-[4-(2,2,2-trifluoroethoxy)phenyl]-2-pyridyl]propan-2-ol (B.1.31), 2-(2,4-difluorophenyl)-1,1-difluoro-3-(tetrazol-1-yl)-1-[5-[4-(trifluoromethoxy)phenyl]-2-pyridyl] propan-2-ol (B.1.32), ipfentrifluconazole (B.1.37), mefentrifluconazole (B.1.38), 2-(chloromethyl)-2-methyl-5-(p-tolylmethyl)-1-(1,2,4-triazol-1-ylmethyl)cyclopentanol (B.1.43); imidazoles: imazalil (B.1.44), pefurazoate (B.1.45), prochloraz (B.1.46), triflumizol (B.1.47); pyrimidines, pyridines, piperazines: fenarimol (B.1.49), pyrifenox (B.1.50), triforine (B.1.51), [3-(4-chloro-2-fluoro-phenyl)-5-(2,4-difluorophenyl)isoxazol-4-yl]-(3-pyridyl)methanol (B.1.52);
   Delta4-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);
   Inhibitors of 3-keto reductase: fenhexamid (B.3.1);
   Other Sterol biosynthesis inhibitors: chlorphenomizole (B.4.1);
C) Nucleic Acid Synthesis Inhibitors
   phenylamides or acyl amino acid fungicides: benalaxyl (C.1.1), benalaxyl-M (C.1.2), kiralaxyl (C.1.3), metalaxyl (C.1.4), metalaxyl-M (C.1.5), ofurace (C.1.6), oxadixyl (C.1.7);
   other nucleic acid synthesis inhibitors: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7), 5-fluoro-2-(4-chlorophenylmethoxy)pyrimidin-4 amine (C.2.8);
D) Inhibitors of Cell Division and Cytoskeleton
   tubulin inhibitors: benomyl (D.1.1), carbendazim (D.1.2), fuberidazole (D1.3), thiabendazole (D.1.4), thiophanate-methyl (D.1.5), pyridachlometyl (D.1.6), N-ethyl-2-[(3-ethynyl-8-methyl-6-quinolyl)oxy]butanamide (D.1.8), N-ethyl-2-[(3-ethynyl-8-methyl-6-quinolyl)oxy]-2-methylsulfanyl-acetamide (D.1.9), 2-[(3-ethynyl-8-methyl-6-quinolyl)oxy]-N-(2-fluoroethyl)butanamide (D.1.10), 2-[(3-ethynyl-8-methyl-6-quinolyl)oxy]-N-(2-fluoroethyl)-2-methoxy-acetamide (D.1.11), 2-[(3-ethynyl-8-methyl-6-quinolyl)oxy]-N-propyl-butanamide (D.1.12), 2-[(3-ethynyl-8-methyl-6-quinolyl)oxy]-2-methoxy-N-propyl-acetamide (D.1.13), 2-[(3-ethynyl-8-methyl-6-quinolyl)oxy]-2-methylsulfanyl-N-propyl-acetamide (D.1.14), 2-[(3-ethynyl-8-methyl-6-quinolyl)oxy]-N-(2-fluoroethyl)-2-methylsulfanyl-acetamide (D.1.15), 4-(2-bromo-4-fluoro-phenyl)-N-(2-chloro-6-fluoro-phenyl)-2,5-dimethyl-pyrazol-3-amine (D.1.16);
   other cell division inhibitors: diethofencarb (D.2.1), ethaboxam (D.2.2), pencycuron (D.2.3), fluopicolide (D.2.4), zoxamide (D.2.5), metrafenone (D.2.6), pyriofenone (D.2.7);
E) Inhibitors of Amino Acid and Protein Synthesis
   methionine synthesis inhibitors: cyprodinil (E.1.1), mepanipyrim (E.1.2), pyrimethanil (E.1.3);
   protein synthesis inhibitors: blasticidin-S(E.2.1), kasugamycin (E.2.2), kasugamycin hydro-chloride-hydrate (E.2.3), mildiomycin (E.2.4), streptomycin (E.2.5), oxytetracyclin (E.2.6);
F) Signal Transduction Inhibitors
   MAP/histidine kinase inhibitors: fluoroimid (F.1.1), iprodione (F.1.2), procymidone (F.1.3), vinclozolin (F.1.4), fludioxonil (F.1.5);
   G protein inhibitors: quinoxyfen (F.2.1);
G) Lipid and Membrane Synthesis Inhibitors
   Phospholipid biosynthesis inhibitors: edifenphos (G.1.1), iprobenfos (G.1.2), pyrazophos (G.1.3), isoprothiolane (G.1.4);
   lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7);
   phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7);
   compounds affecting cell membrane permeability and fatty acides: propamocarb (G.4.1);
   inhibitors of oxysterol binding protein: oxathiapiprolin (G.5.1), 2-{3-[2-(1-{[3,5-bis(difluoro-methyl-1H-pyrazol-1-yl]acetyl}piperidin-4-yl)-1,3-thiazol-4-yl]-4,5-dihydro-1,2-oxazol-5-yl}-phenyl methanesulfonate (G.5.2), 2-{3-[2-(1-{[3,5-bis(difluoromethyl)-1H-pyrazol-1-yl]-acetyl}piperidin-4-yl) 1,3-thiazol-4-yl]-4,5-dihydro-1,2-oxazol-5-yl}-3-chlorophenyl methane-sulfonate (G.5.3), 4-[1-[2-[3-(difluoromethyl)-5-methyl-pyrazol-1-yl]acetyl]-4-piperidyl]-N-tetralin-1-yl-pyridine-2-carboxamide (G.5.4), 4-[1-[2-[3,5-bis(difluoromethyl)pyrazol-1-yl]acetyl]-4-piperidyl]-N-tetralin-1-yl-pyridine-2-carboxamide (G.5.5), 4-[1-[2-[3-(difluoromethyl)-5-(tri-fluoromethyl)pyrazol-1-yl]acetyl]-4-piperidyl]-N-tetralin-1-yl-pyridine-2-carboxamide (G.5.6), 4-[1-[2-[5-cyclopropyl-3-(difluoromethyl)pyrazol-1-yl]acetyl]-4-piperidyl]-N-tetralin-1-yl-pyri-dine-2-carboxamide (G.5.7), 4-[1-[2-[5-methyl-3-(trifluoromethyl)pyrazol-1-yl]acetyl]-4-piperidyl]-N-tetralin-1-yl-pyridine-2-carboxamide (G.5.8), 4-[1-[2-[5-(difluoromethyl)-3-(trifluoro-methyl)pyrazol-1-yl]acetyl]-4-piperidyl]-N-tetralin-1-yl-pyridine-2-carboxamide (G.5.9), 4-[1-[2-[3,5-bis(trifluoromethyl)pyrazol-1-yl]acetyl]-4-piperidyl]-N-tetralin-1-yl-pyridine-2-carboxamide (G.5.10), (4-[1-[2-[5-cyclopropyl-3-(trifluoromethyl)pyrazol-1-yl]acetyl]-4-piperidyl]-N-tetralin-1-yl-pyridine-2-carboxamide (G.5.11);
H) Inhibitors with Multi Site Action
   inorganic active substances: Bordeaux mixture (H.1.1), copper (H.1.2), copper acetate (H.1.3), copper hydroxide (H.1.4), copper oxychloride (H.1.5), basic copper sulfate (H.1.6), sulfur (H.1.7);
   thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
   organochlorine compounds: anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11);
   guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]di-thiino[2,3-c:5,6-c′]dipyrrole-1,3,5,7(2H,6H)-tetraone (H.4.10);
I) Cell Wall Synthesis Inhibitors
   inhibitors of glucan synthesis: validamycin (I.1.1), polyoxin B (1.1.2);
   melanin synthesis inhibitors: pyroquilon (1.2.1), tricyclazole (1.2.2), carpropamid (1.2.3), dicyclomet (1.2.4), fenoxanil (1.2.5);
J) Plant Defense Inducers
   acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), calcium phosphonate (J.1.11), potassium phosphonate (J.1.12), potassium or sodium bicarbonate (J.1.9), 4-cyclopropyl-N-(2,4-dimethoxyphenyl)thiadiazole-5-carboxamide (J.1.10);
K) Unknown Mode of Action
   bronopol (K.1.1), chinomethionat (K.1.2), cyflufenamid (K.1.3), cymoxanil (K.1.4), dazomet (K.1.5), debacarb (K.1.6), diclocymet (K.1.7), diclomezine (K.1.8), difenzoquat (K.1.9), di-fenzoquat-methylsulfate (K.1.10), diphenylamin (K.1.11), fenitropan (K.1.12), fenpyrazamine (K.1.13), flumetover (K.1.14), flusulfamide (K.1.15), flutianil (K.1.16), harpin (K.1.17), methasulfocarb (K.1.18), nitrapyrin (K.1.19), nitrothal-isopropyl (K.1.20), tolprocarb (K.1.21), oxin-copper (K.1.22), proquinazid (K.1.23), tebufloquin (K.1.24), tecloftalam (K.1.25), triazoxide (K.1.26), N′-(4-(4-chloro-3-trifluoromethyl-phenoxy)-2,5-dimethyl-phenyl)-N-ethyl-N-methyl formamidine (K.1.27), N′-(4-(4-fluoro-3-trifluoromethyl-phenoxy)-2,5-dimethyl-phenyl)-N-ethyl-N-methy formamidine (K.1.28), N′-[4-[[3-[(4-chlorophenyl)methyl]-1,2,4-thiadiazol-5-yl]oxy]-2,5-dimethyl-phenyl]-N-ethyl-N-methyl-formamidine (K.1.29), N′(5-bromo-6-indan-2-yloxy-2-methyl-3-pyridyl)-N-ethyl-N-methyl-formamidine (K.1.30), N′-[5-bromo-6-[1-(3,5-difluorophenyl)ethoxy]-2-methyl-3-pyridyl]-N-ethyl-N-methyl-formamidine (K.1.31), N′[5-bromo-6-(4-isopropylcyclohexoxy)-2-methyl-3-pyridyl]-N-ethyl-N-methyl-formamidine (K.1.32), N′[5-bromo-2-methyl-6-(1-phenylethoxy)-3-pyridyl]-N-ethyl-N-methyl-formamidine (K.1.33), N′-(2-methyl-5-trifluoromethyl-4-(3-trimethylsilanyl-propoxy)-phenyl)-N-ethyl-N-methy formamidine (K.1.34), N′(5-difluoromethyl-2-methy-4-(3-trimethylsilanyl-propoxy)-phenyl)-N-ethyl-N-methyl formamidine (K.1.35), 2-(4-chloro-phenyl)-N-[4-(3,4-dimethoxy-phenyl)-isoxazol-5-yl]-2-prop-2-ynyloxy-acetamide (K.1.36), 3-[5-(4-chloro-phenyl)-2,3-dimethyl-isoxazolidin-3-yl]-pyridine (pyrisoxazole) (K.1.37), 3-[5-(4-methylphenyl)-2,3-dimethyl-isoxazolidin-3 yl]-pyridine (K.1.38), 5-chloro-1-(4,6-dimethoxy-pyrimidin-2-yl)-2-methyl-1H-benzoimidazole (K.1.39), ethyl (2-3-amino-2-cyano-3-phenyl-prop-2-enoate (K.1.40), picarbutrazox (K.1.41), pentyl N-[6-[[(2)-[(1-methyltetrazol-5-yl)-phenyl-methylene]amino]oxy-methyl]-2-pyridyl]carbamate (K.1.42), but-3-ynyl N-[6-[[(Z)-[(1-methyltetrazol-5-yl)-phenyl-methylene]amino]oxymethyl]-2-pyridyl]carbamate (K.1.43), ipflufenoquin (K.1.44), quinofumelin (K.1.47), 2-(6-benzyl-2-pyridyl)quinazoline (K.1.50), 2-[6-(3-fluoro-4-methoxy-phenyl)-5-methyl-2-pyridyl]quinazoline (K.1.51), dichlobentiazox (K.1.52), N′-(2,5-dimethyl-4-phenoxy-phenyl)-N-ethyl-N-methyl-formamidine (K.1.53), pyrifenamine (K.1.54);
M) Growth Regulators
   abscisic acid (M.1.1), amidochlor, ancymidol, 6-benzylaminopurine, brassinolide, butralin, chlormequat, chlormequat chloride, choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat, mepiquat chloride, naphthaleneacetic acid, N-6-benzyladenine, paclobutrazol, prohexadione, prohexadione-calcium, prohydrojasmon, thidiazuron, triapenthenol, tributyl phosphorotrithioate, 2,3,5-tri-iodobenzoic acid, trinexapac-ethyl, uniconazole;
N) Herbicides from classes N.1 to N.15
N.1 Lipid biosynthesis inhibitors: alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, 4-(4′-chloro-4-cyclo¬propyl-2′-fluoro[1,1′-biphenyl]-3-yl)-5-hydroxy-2,2,6,6-tetramethyl-2H-pyran-3(6H)-one (1312337-72-6); 4-(2′,4′-dichloro-4-cyclo-propyl [1,1′-biphenyl]-3-yl)-5-hydroxy-2,2,6,6-tetramethyl-2H-pyran-3(6H)-one (1312337-45-3); 4-(4′-chloro-4-ethyl-2′-fluoro[1,1′-biphenyl]-3-yl)-5-hydroxy-2,2,6,6-tetramethyl-2H-pyran-3(6H)-one (1033757-93-5); 4-(2′,4′-dichloro-4-ethyl[1,1′-biphenyl]-3-yl)-2,2,6,6-tetramethyl-2H-pyran-3,5(4H,6H)-dione (1312340-84-3); 5-(acetyloxy)-4-(4′-chloro-4-cyclopropyl-2′-fluoro[1,1′-biphenyl]-3-yl)-3,6-dihydro-2,2,6,6-tetramethyl-2H-pyran-3-one (1312337-48-6); 5-(acetyloxy)-4-(2′,4′-dichloro-4-cyclopropyl-[1,1′-biphenyl]-3-yl)-3,6-dihydro-2,2,6,6-tetra-methyl-2H-pyran-3-one; 5-(acetyloxy)-4-(4′-chloro-4-ethyl-2′-fluoro[1,1′-biphenyl]-3-yl)-3,6-dihydro-2,2,6,6-tetramethyl-2H-pyran-3-one (1312340-82-1); 5-(acetyloxy)-4-(2′,4′-di-chloro-4-ethyl[1,1′-biphenyl]-3-yl)-3,6-dihydro-2,2,6,6-tetramethyl-2H-pyran-3-one (1033760-55-2); 4-(4′-chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (1312337-51-1); 4-(2′,4′-dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (1312340-83-2); 4-(2′,4′-dichloro-4-ethyl¬[1,1′-biphenyl]-3-yl)-5,6-dihydro-2,2,6,6-tetramethyl-5-oxo-2H-pyran-3-yl carbonic acid methyl ester (1033760-58-5); benfuresate, butylate, cycloate, dalapon, dimepiperate, EPTC, esprocarb, ethofumesate, flupropanate, molinate, orbencarb, pebulate, prosulfocarb, TCA, thiobencarb, tiocarbazil, triallate, vernolate;
N.2 ALS inhibitors: amidosulfuron, azimsulfuron, bensulfuron, bensulfuron-methyl, chlorimuron, chlorimuron-ethyl, chlorsulfuron, cinosulfuron, cyclosulfamuron, ethametsulfuron, ethametsulfuron-methyl, ethoxysulfuron, flazasulfuron, flucetosulfuron, flupyrsulfuron, flupyrsulfuron-methyl-sodium, foramsulfuron, halosulfuron, halosulfuronmethyl, imazosulfuron, iodosulfuron, iodosulfuron-methyl-sodium, iofensulfuron, iofensulfuron-sodium, mesosulfuron, metazosulfuron, metsulfuron, metsulfuron-methyl, nicosulfuron, orthosulfamuron, oxasulfuron, primisulfuron, primisulfuron-methyl, propyrisulfuron, prosulfuron, pyrazosulfuron, pyrazosulfuron-ethyl, rimsulfuron, sulfometuron, sulfometuron-methyl, sulfosulfuron, thifensulfuron, thifensulfuron-methyl, triasulfuron, tribenuron, tribenuronmethyl, trifloxysulfuron, triflusulfuron, triflusulfuron-methyl, tritosulfuron, imazamethabenz, imazamethabenz-methyl, imazamox, imazapic, imazapyr, imazaquin, imazethapyr; cloransulam, cloransulam-methyl, diclosulam, flumetsulam, florasulam, metosulam, penoxsulam, pyrimisulfan, pyroxsulam; bispyribac, bispyribac-sodium, pyribenzoxim, pyriftalid, pyriminobac, pyriminobac-methyl, pyrithiobac, pyrithiobac-sodium, 4-[[[2-[(4,6-dimethoxy-2-pyrimidinyl)oxy]phenyl]methyl]amino]-benzoic acid-1-methyl-ethyl ester (420138-41-6), 4-[[[2-[(4,6-dimethoxy-2-pyrimidinyl)oxy]phenyl] methyl]amino]-benzoic acid propyl ester (420138-40-5), N-(4-bromophenyl)-2-[(4,6-dimethoxy-2-pyrimidinyl)oxy]-benzenemethanamine (420138-01-8); flucarbazone, flucarbazone-sodium, propoxycarbazone, propoxycarbazone-sodium, thiencarbazone, thiencarbazone-methyl; triafamone;
N.3 Photosynthesis inhibitors: amicarbazone; chlorotriazine; ametryn, atrazine, chloridazone, cyanazine, desmetryn, dimethametryn, hexazinone, metribuzin, prometon, prometryn, propazine, simazine, simetryn, terbumeton, terbuthylazin, terbutryn, trietazin; chlorobromuron, chlorotoluron, chloroxuron, dimefuron, diuron, fluometuron, isoproturon, isouron, linuron, metamitron, methabenzthiazuron, metobenzuron, metoxuron, monolinuron, neburon, siduron, tebuthiuron, thiadiazuron, desmedipham, karbutilat, phenmedipham, phenmediphamethyl, bromofenoxim, bromoxynil and its salts and esters, ioxynil and its salts and esters, bromacil, lenacil, terbacil, bentazon, bentazon-sodium, pyridate, pyridafol, pentanochlor, propanil; diquat, diquat-dibromide, paraquat, paraquat-dichloride, paraquat-dimetilsulfate;
N.4 protoporphyrinogen-IX oxidase inhibitors: acifluorfen, acifluorfen-sodium, azafenidin, bencarbazone, benzfendizone, bifenox, butafenacil, carfentrazone, carfentrazone-ethyl, chlormethoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, tiafenacil, trifludimoxazin, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (353292-31-6), N-ethyl-3-(2,6-dichloro-4-trifluoro-methylphenoxy)-5-methyl-1H-pyrazole-1-carboxamide (452098-92-9), N-tetrahydrofurfuryl-3-(2,6-dichloro-4-trifluoromethylphenoxy)-5-methyl-1H-pyrazole-1-carboxamide (915396-43-9), N-ethyl-3-(2-chloro-6-fluoro-4-trifluoromethyl phenoxy)-5-methyl-1H-pyrazole-1-carboxamide (452099-05-7), N-tetrahydro-furfuryl-3-(2-chloro-6-fluoro-4-trifluoro¬methylphenoxy)-5-methyl-1H-pyrazole-1-carboxamide (452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (1300118-96-0), 1-methyl-6-trifluoro¬methyl-3-(2,2,7-tri-fluoro-3-oxo-4-prop-2-ynyl-3,4-dihydro-2H-benzo[1,4]oxazin-6-yl)-1H-pyrimidine-2,4-dione (1304113-05-0), methyl (E)-4-[2-chloro-5-[4-chloro-5-(difluoromethoxy)-1H-methyl-pyrazol-3-yl]-4-fluoro-phenoxy]-3-methoxy-but-2-enoate (948893-00-3), 3-[7-chloro-5-fluoro-2-(trifluoromethyl)-1H-benzimidazol-4-yl]-1-methyl-6-(trifluoromethyl)-1H-pyrimidine-2,4-dione (212754-02-4);
N.5 Bleacher herbicides: beflubutamid, diflufenican, fluridone, flurochloridone, flurtamone, norflurazon, picolinafen, 4-(3-trifluoromethyl¬phenoxy)-2-(4-trifluoromethylphenyl)pyrimidine (180608-33-7); benzobicyclon, benzofenap, bicyclopyrone, clomazone, fenquintrione, isoxaflutole, mesotrione, pyrasulfotole, pyrazolynate, pyrazoxyfen, sulcotrione, tefuryltrione, tembotrione, tolpyralate, topramezone; aclonifen, amitrole, flumeturon;
N.6 EPSP synthase inhibitors: glyphosate, glyphosate-isopropylammonium, glyposate-potassium, glyphosate-trimesium (sulfosate);
N.7 Glutamine synthase inhibitors: bilanaphos (bialaphos), bilanaphos-sodium, glufosinate, glufosinate-P, glufosinate-ammonium;
N.8 DHP synthase inhibitors: asulam;
N.9 Mitosis inhibitors: benfluralin, butralin, dinitramine, ethalfluralin, fluchloralin, oryzalin, pendimethalin, prodiamine, trifluralin; amiprophos, amiprophos-methyl, butamiphos; chlorthal, chlorthal-dimethyl, dithiopyr, thiazopyr, propyzamide, tebutam; carbetamide, chlorpropham, flamprop, flamprop-isopropyl, flamprop-methyl, flamprop-M-isopropyl, flamprop-M-methyl, propham;
N.10 VLCFA inhibitors: acetochlor, alachlor, butachlor, dimethachlor, dimethenamid, dimethenamid-P, metazachlor, metolachlor, metolachlor-S, pethoxamid, pretilachlor, propachlor, propisochlor, thenylchlor, flufenacet, mefenacet, diphenamid, naproanilide, napropamide, napropamide-M, fentrazamide, anilofos, cafenstrole, fenoxasulfone, ipfencarbazone, piperophos, pyroxasulfone, isoxazoline compounds of the formulae II.1, II.2, II.3, II.4, II.5, II.6, II.7, II.8 and II.9

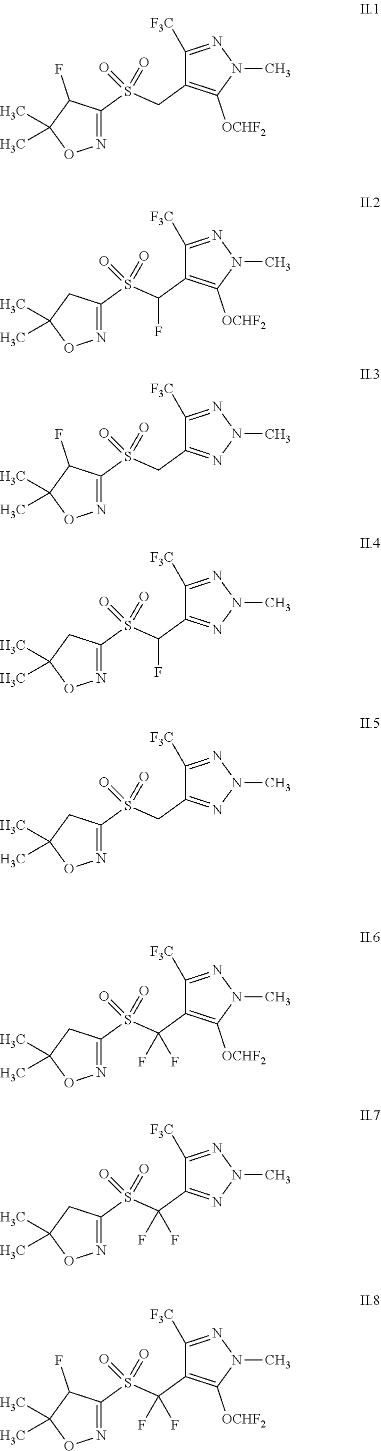

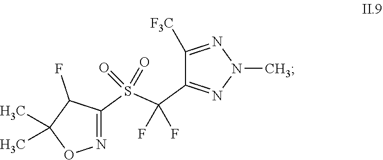 [SMILES: Cn1nc(C(F)(F)F)c(C(F)(F)S(=O)(=O)C2=NOC(C)(C)C2F)n1]

N.11 Cellulose biosynthesis inhibitors: chlorthiamid, dichlobenil, flupoxam, indaziflam isoxaben, triaziflam, 1-cyclohexyl-5-pentafluorphenyloxy-14-[1,2,4,6]thiatriazin-3-ylamine (175899-01-1);
N.12 Decoupler herbicides: dinoseb, dinoterb, DNOC and its salts;
N.13 Auxinic herbicides: 2,4-D and its salts and esters, clacyfos, 2,4-DB and its salts and esters, aminocyclopyrachlor and its salts and esters, aminopyralid and its salts such as aminopyralid-dimethylammonium, aminopyralid-tris(2-hydroxypropyl)ammonium and its esters, benazolin, benazolin-ethyl, chloramben and its salts and esters, clomeprop, clopyralid and its salts and esters, dicamba and its salts and esters, dichlorprop and its salts and esters, dichlorprop-P and its salts and esters, fluroxypyr, fluroxypyr-butometyl, fluroxypyr-meptyl, halauxifen and its salts and esters (943832-60-8); MCPA and its salts and esters, MCPA-thioethyl, MCPB and its salts and esters, mecoprop and its salts and esters, mecoprop-P and its salts and esters, picloram and its salts and esters, quinclorac, quinmerac, TBA (2,3,6) and its salts and esters, triclopyr and its salts and esters, 4-amino-3-chloro-6-(4-chloro-2-fluoro-3-methoxyphenyl)-5-fluoropyridine-2-carboxylic acid, benzyl 4-amino-3-chloro-6-(4-chloro-2-fluoro-3-methoxyphenyl)-5-fluoropyridine-2-carboxylate (1390661-72-9);
N.14 Auxin transport inhibitors: diflufenzopyr, diflufenzopyr-sodium, naptalam, naptalam-sodium;
N.15 Other herbicides: bromobutide, chlorflurenol, chlorflurenol-methyl, cinmethylin, cumyluron, cyclopyrimorate (499223-49-3) and its salts and esters, dalapon, dazomet, difenzoquat, difenzoquat-metilsulfate, dimethipin, DSMA, dymron, endothal and its salts, etobenzanid, flurenol, flurenol-butyl, flurprimidol, fosamine, fosamine-ammonium, indanofan, maleic hydrazide, mefluidide, metam, methiozolin (403640-27-7), methyl azide, methyl bromide, methyl-dymron, methyl iodide, MSMA, oleic acid, oxaziclomefone, pelargonic acid, pyributicarb, quinoclamine, tridiphane;
O) Insecticides from classes 0.1 to O.29
O.1 Acetylcholine esterase (AChE) inhibitors: aldicarb (O.1.1), alanycarb (O.1.2), bendiocarb (O.1.3), benfuracarb (O.1.4), butocarboxim (O.1.5), butoxycarboxim (O.1.6), carbaryl (O.1.7), carbofuran (O.1.8), carbosulfan (O.1.9), ethiofencarb (O.1.10), fenobucarb (O.1.11), formetanate (O.1.12), furathiocarb (O.1.13), isoprocarb (O.1.14), methiocarb (O.1.15), methomyl (O.1.16), metolcarb (O.1.17), oxamyl (O.1.18), pirimicarb (O.1.19), propoxur (O.1.20), thiodicarb (O.1.21), thiofanox (O.1.22), trimethacarb (O.1.23), XMC (O.1.24), xylylcarb (O.1.25), triazamate (O.1.26), acephate (O.1.27), azamethiphos (O.1.28), azinphos-ethyl (O.1.29), azinphosmethyl (O.1.30), cadusafos (O.1.31), chlorethoxyfos (O.1.32), chlorfenvinphos (O.1.33), chlormephos (O.1.34), chlorpyrifos (O.1.35), chlorpyrifos-methyl (O.1.36), coumaphos (O.1.37), cyanophos (O.1.38), demeton-S-methyl (O.1.39), diazinon (O.1.40), dichlorvos/DDVP (O.1.41), dicrotophos (O.1.42), dimethoate (O.1.43), dimethylvinphos (O.1.44), disulfoton (O.1.45), EPN (O.1.46), ethion (O.1.47), ethoprophos (O.1.48), famphur (O.1.49), fenamiphos (O.1.50), fenitrothion (O.1.51), fenthion (O.1.52), fosthiazate (O.1.53), heptenophos (O.1.54), imicyafos (O.1.55), isofenphos (O.1.56), isopropyl O-(methoxyaminothio-phosphoryl) salicylate (O.1.57), isoxathion (O.1.58), malathion (O.1.59), mecarbam (O.1.60), methamidophos (O.1.61), methidathion (O.1.62), mevinphos (O.1.63), monocrotophos (O.1.64), naled (O.1.65), omethoate (O.1.66), oxydemeton-methyl (O.1.67), parathion (O.1.68), parathion-methyl (O.1.69), phenthoate (O.1.70), phorate (O.1.71), phosalone (O.1.72), phosmet (O.1.73), phosphamidon (O.1.74), phoxim (O.1.75), pirimiphos-methyl (O.1.76), profenofos (O.1.77), propetamphos (O.1.78), prothiofos (O.1.79), pyraclofos (O.1.80), pyridaphenthion (O.1.81), quinalphos (O.1.82), sulfotep (O.1.83), tebupirimfos (O.1.84), temephos (O.1.85), terbufos (O.1.86), tetrachlorvinphos (O.1.87), thiometon (O.1.88), triazophos (O.1.89), trichlorfon (O.1.90), vamidothion (O.1.91);
O.2 GABA-gated chloride channel antagonists: endosulfan (O.2.1), chlordane (O.2.2), ethiprole (O.2.3), fipronil (O.2.4), flufiprole (O.2.5), pyrafluprole (O.2.6), pyriprole (O.2.7);
O.3 Sodium channel modulators: acrinathrin (O.3.1), allethrin (O.3.2), d-cis-trans allethrin (O.3.3), d-trans allethrin (O.3.4), bifenthrin (O.3.5), kappa-bifenthrin (O.3.6), bioallethrin (O.3.7), bioallethrin S-cylclopentenyl (O.3.8), bioresmethrin (O.3.9), cycloprothrin (O.3.10), cyfluthrin (O.3.11), beta-cyfluthrin (O.3.12), cyhalothrin (O.3.13), lambda-cyhalothrin (O.3.14), gamma-cyhalothrin (O.3.15), cypermethrin (O.3.16), alpha-cypermethrin (O.3.17), beta-cypermethrin (O.3.18), theta-cypermethrin (O.3.19), zeta-cypermethrin (O.3.20), cyphenothrin (O.3.21), deltamethrin (O.3.22), empenthrin (O.3.23), esfenvalerate (O.3.24), etofenprox (O.3.25), fenpropathrin (O.3.26), fenvalerate (O.3.27), flucythrinate (O.3.28), flumethrin (O.3.29), tau-fluvalinate (O.3.30), halfenprox (O.3.31), heptafluthrin (O.3.32), imiprothrin (O.3.33), meperfluthrin (O.3.34), metofluthrin (O.3.35), momfluorothrin (O.3.36), epsilon-momfluorothrin (O.3.37), permethrin (O.3.38), phenothrin (O.3.39), prallethrin (O.3.40), profluthrin (O.3.41), pyrethrin (pyrethrum) (O.3.42), resmethrin (O.3.43), silafluofen (O.3.44), tefluthrin (O.3.45), kappa-tefluthrin (O.3.46), tetramethylfluthrin (O.3.47), tetramethrin (O.3.48), tralomethrin (O.3.49), transfluthrin (O.3.50), DDT (O.3.51), methoxychlor (O.3.52);
O.4 Nicotinic acetylcholine receptor agonists (nAChR): acetamiprid (O.4.1), clothianidin (O.4.2), cycloxaprid (O.4.3), dinotefuran (O.4.4), imidacloprid (O.4.5), nitenpyram (O.4.6), thiacloprid (O.4.7), thiamethoxam (O.4.8), 4,5-dihydro-N-nitro-1-(2-oxiranylmethyl)-1H-imidazol-2-amine (O.4.9), (2E)-1-[(6-chloropyridin-3-yl)methyl]-N′-nitro-2-pentylidene-hydrazinecarboximidamide (O.4.10), 1-[(6-chloropyridin-3-yl)methyl]-7-methyl-8-nitro-5-propoxy-1,2,3,5,6,7-hexahydroimidazo[1,2-a]pyridine (O.4.11), nicotine (O.4.12), sulfoxaflor (O.4.13), flupyradifurone (O.4.14), triflumezopyrim (O.4.15);
O.5 Nicotinic acetylcholine receptor allosteric activators: spinosad (O.5.1), spinetoram (O.5.2);
O.6 Chloride channel activators: abamectin (O.6.1), emamectin benzoate (O.6.2), ivermectin (O.6.3), lepimectin (O.6.4), milbemectin (O.6.5);
O.7 Juvenile hormone mimics: hydroprene (O.7.1), kinoprene (O.7.2), methoprene (O.7.3), fenoxycarb (O.7.4), pyriproxyfen (O.7.5);
O.8 miscellaneous non-specific (multi-site) inhibitors: methyl bromide (O.8.1) and other alkyl halides, chloropicrin (O.8.2), sulfuryl fluoride (O.8.3), borax (O.8.4), tartar emetic (O.8.5);
O.9 Chordotonal organ TRPV channel modulators: pymetrozine (O.9.1), pyrifluquinazon (O.9.2), flonicamid (O.9.3);
O.10 Mite growth inhibitors: clofentezine (O.10.1), hexythiazox (O.10.2), diflovidazin (O.10.3), etoxazole (O.10.4);
O.11 Microbial disruptors of insect midgut membranes: Bacillus thuringiensis, Bacillus sphaericus and the insecticdal proteins they produce: Bacillus thuringiensis subsp. Israelensis (O.11.1), Bacillus sphaericus (O.11.2), Bacillus thuringiensis subsp. aizawai (O.11.3), Bacillus thuringiensis subsp. kurstaki (O.11.4), Bacillus thuringiensis subsp. tenebrionis (O.11.5), the Bt crop proteins: Cry1Ab (O.11.6), Cry1Ac (O.11.7), Cry1Fa (O.11.8), Cry2Ab (O.11.9), mCry3A (O.11.10), Cry3Ab (O.11.11), Cry3Bb (O.11.12), Cry34/35Ab1 (O.11.13);
O.12 Inhibitors of mitochondrial ATP synthase: diafenthiuron (O.12.1), azocyclotin (O.12.2), cyhexatin (O.12.3), fenbutatin oxide (O.12.4), propargite (O.12.5), tetradifon (O.12.6);
O.13 Uncouplers of oxidative phosphorylation via disruption of the proton gradient: chlorfenapyr (O.13.1), DNOC (O.13.2), sulfluramid (O.13.3);
O.14 Nicotinic acetylcholine receptor (nAChR) channel blockers: bensultap (O.14.1), cartap hydrochloride (O.14.2), thiocyclam (O.14.3), thiosultap sodium (O.14.4);
O.15 Inhibitors of the chitin biosynthesis type 0: bistrifluron (O.15.1), chlorfluazuron (O.15.2), diflubenzuron (O.15.3), flucycloxuron (O.15.4), flufenoxuron (O.15.5), hexaflumuron (O.15.6), lufenuron (O.15.7), novaluron (O.15.8), noviflumuron (O.15.9), teflubenzuron (O.15.10), triflumuron (O.15.11);
O.16 Inhibitors of the chitin biosynthesis type 1: buprofezin (O.16.1);
O.17 Moulting disruptors: cyromazine (O.17.1);
O.18 Ecdyson receptor agonists: methoxyfenozide (O.18.1), tebufenozide (O.18.2), halofenozide (O.18.3), fufenozide (O.18.4), chromafenozide (O.18.5);
O.19 Octopamin receptor agonists: amitraz (O.19.1);
O.20 Mitochondrial complex III electron transport inhibitors: hydramethylnon (O.20.1), acequinocyl (O.20.2), fluacrypyrim (O.20.3), bifenazate (O.20.4);
O.21 Mitochondrial complex I electron transport inhibitors: fenazaquin (O.21.1), fenpyroximate (O.21.2), pyrimidifen (O.21.3), pyridaben (O.21.4), tebufenpyrad (O.21.5), tolfenpyrad (O.21.6), rotenone (O.21.7);
O.22 Voltage-dependent sodium channel blockers: indoxacarb (O.22.1), metaflumizonev (O.22.2), 2-[2-(4-cyanophenyl)-1-[3-(trifluoromethyl)phenyl]ethylidene]-N-[4-(difluoromethoxy)phenyl]-hydrazinecarboxamide (O.22.3), N-(3-chloro-2-methylphenyl)-2-[(4-chlorophenyl)-[4-[methyl(methylsulfonyl)amino]phenyl]methylene]-hydrazinecarboxamide (O.22.4);
O.23 Inhibitors of the of acetyl CoA carboxylase: spirodiclofen (O.23.1), spiromesifen (O.23.2), spirotetramat (O.23.3), spiropidion (O.23.4);
O.24 Mitochondrial complex IV electron transport inhibitors: aluminium phosphide (O.24.1), calcium phosphide (O.24.2), phosphine (O.24.3), zinc phosphide (O.24.4), cyanide (O.24.5);
O.25 Mitochondrial complex II electron transport inhibitors: cyenopyrafen (O.25.1), cyflumetofen (O.25.2);
O.26 Ryanodine receptor-modulators: flubendiamide (O.26.1), chlorantraniliprole (O.26.2), cyantraniliprole (O.26.3), cyclaniliprole (O.26.4), tetraniliprole (O.26.5), (R)-3-chloro-N-{2-methyl-4-[1,2,2,2-tetrafluoro-1-(trifluoromethyl)ethyl]phenyl}-N²-(1-methyl-2-methylsulfonylethyl)phthalamide (O.26.6), (S)-3-chloro-N¹-{2-methyl-4-[1,2,2,2-tetrafluoro-1-(trifluoromethyl)ethyl]phenyl}-M-(1-methyl-2-methylsulfonylethyl)phthalamide (O.26.7), methyl-2-[3,5-dibromo-2-({[3-bromo-1-(3-chloropyridin-2-yl)-1H-pyrazol-5-yl]carbonyl}-amino)benzoyl]-1,2-dimethylhydrazinecarboxylate (O.26.8), N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide (O.26.9), N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide (O.26.10), N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide (O.26.11), N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide (O.26.12), N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide (O.26.13), N-[2-(5-amino-1,3,4-thiadiazol-2-yl)-4-chloro-6-methylphenyl]-3-bromo-1-(3-chloro-2-pyridinyl)-1H-pyrazole-5-carboxamide (O.26.14), 3-chloro-1-(3-chloro-2-pyridinyl)-N-[2,4-dichloro-6-[[(1-cyano-1-methylethyl)amino]carbonyl]phenyl]-1H-pyrazole-5-carboxamide (O.26.15), 3-bromo-N-[2,4-dichloro-6-(methylcarbamoyl)phenyl]-1-(3,5-dichloro-2-pyridyl)-1H-pyrazole-5-carboxamide (O.26.16), N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide (O.26.17), cyhalodiamide (O.26.18);
O.27: Chordotonal organ Modulators—undefined target site: flonicamid (O.27.1);
O.28. insecticidal active compounds of unknown or uncertain mode of action: afidopyropen (O.28.1), afoxolaner (O.28.2), azadirachtin (O.28.3), amidoflumet (O.28.4), benzoximate (O.28.5), broflanilide (O.28.6), bromopropylate (O.28.7), chinomethionat (O.28.8), cryolite (O.28.9), dicloromezotiaz (O.28.10), dicofol (O.28.11), flufenerim (O.28.12), flometoquin (O.28.13), fluensulfone (O.28.14), fluhexafon (O.28.15), fluopyram (O.28.16), fluralaner (O.28.17), metoxadiazone (O.28.18), piperonyl butoxide (O.28.19), pyflubumide (O.28.20), pyridalyl (O.28.21), tioxazafen (O.28.22), 11-(4-chloro-2,6-dimethylphenyl)-12-hydroxy-1,4-dioxa-9-azadispiro[4.2.4.2]-tetradec-11-en-10-one, 3-(4′-fluoro-2,4-dimethylbiphenyl-3-yl)-4-hydroxy-8-oxa-1-azaspiro[4.5]dec-3-en-2-one, 1-[2-fluoro-4-methyl-5-[(2,2,2-trifluoroethyl)sulfinyl]phenyl]-3-(trifluoromethyl)-1H-1,2,4-triazole-5-amine (O.28.23), Bacillus firmus I-1582 (O.28.24), flupyrimin (O.28.25), fluazaindolizine (O.28.26), 4-[5-(3,5-di-chlorophenyl)-5-(trifluoromethyl)-4H-isoxazol-3-yl]-2-methyl-N-(1-oxothietan-3-yl)benzamide (O.28.27), fluxametamide (O.28.28), 5-[3-[2,6-dichloro-4-(3,3-dichloroallyloxy)phenoxy]propoxy]-1H-pyrazole (O.28.1), 4-cyano-N-[2-cyano-5-[[2,6-dibromo-4-[1,2,2,3,3,3-hexafluoro-1-(trifluoromethyl)propyl] phenyl]carbamoyl] phenyl]-2-methyl-benzamide (O.28.29), 4-cyano-3-[(4-cyano-2-methyl-benzoyl)amino]-N-[2,6-di-chloro-4-[1,2,2,3,3,3-hexafluoro-1-(trifluoromethyl)propyl]phenyl]-2-fluoro-benzamide (O.28.30), N-[5-[[2-chloro-6-cyano-4-[1,2,2,3,3,3-hexafluoro-1-(trifluoromethyl)propyl]-phenyl]carbamoyl]-2-cyano-phenyl]-4-cyano-2-methyl-benzamide (O.28.31), N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide (O.28.32), N-[5-[[2-bromo-6-chloro-4-[1,2,2,3,3,3-hexafluoro-1-(trifluoromethyl)propyl] phenyl]carbamoyl]-2-cyano-phenyl]-4-cyano-2-methyl-benzamide (O.28.33), 4-cyano-N-[2-cyano-5-[[2,6-dichloro-4-[1,2,2,3,3,3-hexafluoro-1-(trifluoromethyl)propyl]phenyl]carbamoyl]phenyl]-2-methyl-benzamide (O.28.34), 4-cyano-N-[2-cyano-5-[[2,6-dichloro-4-[1,2,2,2-tetrafluoro-1-(trifluoromethyl)ethyl] phenyl]carbamoyl]phenyl]-2-methyl-benzamide (O.28.35), N-[5-[[2-bromo-6-chloro-4-[1,2,2,2-tetrafluoro-1-(trifluoromethyl)ethyl]phenyl]carbamoyl]-2-cyano-phenyl]-4-cyano-2-methyl-benzamide (O.28.36); 2-(1,3-dioxan-2-yl)-6-[2-(3-pyridinyl)-5-thiazolyl]-pyridine (O.28.37), 2-[6-[2-(5-fluoro-3-pyridinyl)-5-thiazolyl]-2-pyridinyl]-pyrimidine (O.28.38), 2-[6-[2-(3-pyridinyl)-5-thiazolyl]-2-pyridinyl]-pyrimidine (O.28.39), N-methylsulfonyl-6-[2-(3-pyridyl)thiazol-5-yl]pyridine-2-carboxamide (O.28.40), N-methylsulfonyl-6-[2-(3-pyridyl)thiazol-5-yl]pyridine-2-carboxamide (O.28.41), 1-[(6-chloro-3-pyridinyl)methyl]-1,2,3,5,6,7-hexahydro-5-methoxy-7-methyl-8-nitro-imidazo[1,2-a] pyridine (O.28.42), 1-[(6-chloropyridin-3-yl)methyl]-7-methyl-8-nitro-1,2,3,5,6,7-hexahydroimidazo[1,2-a]pyridin-5-ol (O.28.43), 1-isopropyl-N,5-dimethyl-N-pyridazin-4-yl-pyrazole-4-carboxamide (O.28.44), 1-(1,2-dimethylpropyl)-N-ethyl-5-methyl-N-pyridazin-4-yl-pyrazole-4-carboxamide (O.28.45), N,5-dimethyl-N-pyridazin-4-yl-1-(2,2,2-trifluoro-1-methyl-ethyl)pyrazole-4-carboxamide (O.28.46), 1-[1-(1-cyanocyclopropyl)ethyl]-N-ethyl-5-methyl-N-pyridazin-4-yl-pyrazole-4-carboxamide (O.28.47), N-ethyl-1-(2-fluoro-1-methyl-propyl)-5-meth-yl-N-pyridazin-4-yl-pyrazole-4-carboxamide (O.28.48), 1-(1,2-dimethylpropyl)-N,5-dimethyl-N-pyridazin-4-yl-pyrazole-4-carboxamide (O.28.49), 1-[1-(1-cyanocyclopropyl)ethyl]-N,5-di-methyl-N-pyridazin-4-yl-pyrazole-4-carboxamide (O.28.50), N-methyl-1-(2-fluoro-1-methyl-propyl]-5-methyl-N-pyridazin-4-yl-pyrazole-4-carboxamide (O.28.51), 1-(4,4-di-fluorocyclohexyl)-N-ethyl-5-methyl-N-pyridazin-4-yl-pyrazole-4-carboxamide (O.28.52), 1-(4,4-difluorocyclohexyl)-N,5-dimethyl-N-pyridazin-4-yl-pyrazole-4-carboxamide (O.28.53), N-(1-methylethyl)-2-(3-pyridinyl)-2H-indazole-4-carboxamide (O.28.54), N-cyclopropyl-2-(3-pyridinyl)-2H-indazole-4-carboxamide (O.28.55), N-cyclohexyl-2-(3-pyridinyl)-2H-indazole-4-carboxamide (O.28.56), 2-(3-pyridinyl)-N-(2,2,2-trifluoroethyl)-2H-indazole-4-carboxamide (O.28.57), 2-(3-pyridinyl)-N-[(tetrahydro-2-furanyl)methyl]-2H-indazole-5-carboxamide (O.28.58), methyl 2-[[2-(3-pyridinyl)-2H-indazol-5-yl]carbonyl]hydrazinecarboxylate (O.28.59), N-[(2,2-difluorocyclopropyl)methyl]-2-(3-pyridinyl)-2H-indazole-5-carboxamide (O.28.60), N-(2,2-difluoropropyl)-2-(3-pyridinyl)-2H-indazole-5-carboxamide (O.28.61), 2-(3-pyridinyl)-N-(2-pyrimidinylmethyl)-2H-indazole-5-carboxamide (O.28.62), N-[(5-methyl-2-pyrazinyl)methyl]-2-(3-pyridinyl)-2H-indazole-5-carboxamide (O.28.63), tyclopyrazoflor (O.28.64), sarolaner (O.28.65), lotilaner (O.28.66), N-[4-chloro-3-[[(phenyl methyl)amino]carbonyl] phenyl]-1-methyl-3-(1,1,2,2,2-penta-fluoroethyl)-4-(trifluoromethyl)-1H-pyrazole-5-carboxamide (O.28.67), M.UN.22a 2-(3-ethylsulfonyl-2-pyridyl)-3-methyl-6-(trifluoromethyl)imidazo[4,5-b]pyridine (O.28.68), 2-[3-ethylsuIfonyl-5-(trifluoromethyl)-2-pyridyl]-3-methyl-6-(trifluoromethyl)imidazo[4,5-b]pyridine (O.28.69), 4-[5-(3,5-dichlorophenyl)-5-(trifluoromethyl)-4H-isoxazol-3-yl]-N-[(4R)-2-ethyl-3-oxo-isoxazolidin-4-yl]-2-methyl-benzamide (O.28.70), 4-[5-(3,5-dichloro-4-fluoro-phenyl)-5-(trifluoromethyl)-4H-isoxazol-3-yl]-N-[(4R)-2-ethyl-3-oxo-isoxazolidin-4-yl]-2-methyl-benzamide (O.28.71), N-[4-chloro-3-(cyclopropylcarbamoyl)phenyl]-2-methyl-5-(1,1,2,2,2-pentafluoroethyl)-4-(trifluoromethyl)pyrazole-3-carboxamide (O.28.72), N-[4-chloro-3-[(1-cyanocyclopropyl)carbamoyl] phenyl]-2-methyl-5-(1,1,2,2,2-pentafluoroethyl)-4-(trifluoromethyl)pyrazole-3-carboxamide (O.28.73), acynonapyr (O.28.74), benzpyrimoxan (O.28.75), chloro-N-(1-cyanocyclopropyl)-5-[1-[2-methyl-5-(1,1,2,2,2-pentafluoroethyl)-4-(trifluoromethyl)pyrazol-3-yl]pyrazol-4-yl]benzamide (O.28.76), oxazosulfyl (O.28.77), [(2S,3R,4R,5S,6S)-3,5-di methoxy-6-methyl-4-propoxy-tetrahydropyran-2-yl]-N-[4-[1-[4-(tri-fluoromethoxy)phenyl]-1,2,4-triazol-3-yl]phenyl]carbamate (O.28.78), [(2S,3R,4R,5S,6S)-3,4,5-trimethoxy-6-methyl-tetrahydropyran-2-yl] N-[4-[1-[4-(trifluoromethoxy)phenyl]-1,2,4-triazol-3-yl]phenyl]carbamate (O.28.79), [(2S,3R,4R,5S,6S)-3,5-dimethoxy-6-methyl-4-propoxy-tetrahydropyran-2-yl]-N-[4-[1-[4-(1,1,2,2,2-pentafluoroethoxy)phenyl]-1,2,4-triazol-3-yl]phenyl]carbamate (O.28.80), [(2S,3R,4R,5S,6S)-3,4,5-trimethoxy-6-methyl-tetrahydropyran-2-yl]-N-[4-[1-[4-(1,1,2,2,2-pentafluoroethoxy)phenyl]-1,2,4-triazol-3-yl]phenyl]carbamate (O.28.81), (22-3-(2-isopropylphenyl)-2-[(E)-[4-[1-[4-(1,1,2,2,2-pentafluoroethoxy)phenyl]-1,2,4-triazol-3-yl]phenyl] methylenehydrazono]thiazolidin-4-one (O.28.82).

The active substances referred to as component 2, their preparation and their activity e.g. against harmful fungi is known (cf.: http://www.alanwood.net/pesticides/); these substances are commercially available. The compounds described by IUPAC nomenclature, their preparation and their pesticidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. Nos. 3,296,272; 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 10/139271, WO 11/028657, WO 12/168188, WO 07/006670, WO 11/77514; WO 13/047749, WO 10/069882, WO 13/047441, WO 03/16303, WO 09/90181, WO 13/007767, WO 13/010862, WO 13/127704, WO 13/024009, WO 13/24010, WO 13/047441, WO 13/162072, WO 13/092224, WO 11/135833, CN 1907024, CN 1456054, CN 103387541, CN 1309897, WO 12/84812, CN 1907024, WO 09094442, WO 14/60177, WO 13/116251, WO 08/013622, WO 15/65922, WO 94/01546, EP 2865265, WO 07/129454, WO 12/165511, WO 11/081174, WO 13/47441).

The present invention furthermore relates to agrochemical compositions comprising a mixture of at least one compound I (component 1) and at least one further active substance useful for plant protection, e.g. selected from the groups A) to O) (component 2), in particular one further fungicide, e.g. one or more fungicide from the groups A) to K), as described above, and if desired one suitable solvent or solid carrier. Those mixtures are of particular interest, since many of them at the same application rate show higher efficiencies against harmful fungi. Furthermore, combating harmful fungi with a mixture of compounds I and at least one fungicide from groups A) to K), as described above, is more efficient than combating those fungi with individual compounds I or individual fungicides from groups A) to K).

By applying compounds I together with at least one active substance from groups A) to O) a synergistic effect can be obtained, i.e. more then simple addition of the individual effects is obtained (synergistic mixtures).

This can be obtained by applying the compounds I and at least one further active substance simultaneously, either jointly (e.g. as tank-mix) or separately, or in succession, wherein the time interval between the individual applications is selected to ensure that the active substance applied first still occurs at the site of action in a sufficient amount at the time of application of the further active substance(s). The order of application is not essential for working of the present invention.

When applying compound I and a pesticide II sequentially the time between both applications may vary e.g. between 2 hours to 7 days. Also a broader range is possible ranging from 0.25 hour to 30 days, preferably from 0.5 hour to 14 days, particularly from 1 hour to 7 days or from 1.5 hours to 5 days, even more preferred from 2 hours to 1 day. In case of a mixture comprising a pesticide II selected from group L), it is preferred that the pesticide II is applied as last treatment.

In the binary mixtures and compositions according to the invention the weight ratio of the component 1) and the component 2) generally depends from the properties of the active components used, usually it is in the range of from 1:10,000 to 10,000:1, often it is in the range of from 1:100 to 100:1, regularly in the range of from 1:50 to 50:1, preferably in the range of from 1:20 to 20:1, more preferably in the range of from 1:10 to 10:1, even more preferably in the range of from 1:4 to 4:1 and in particular in the range of from 1:2 to 2:1.

According to further embodiments of the binary mixtures and compositions, the weight ratio of the component 1) and the component 2) usually is in the range of from 1000:1 to 1:1, often in the range of from 100:1 to 1:1, regularly in the range of from 50:1 to 1:1, preferably in the range of from 20:1 to 1:1, more preferably in the range of from 10:1 to 1:1, even more preferably in the range of from 4:1 to 1:1 and in particular in the range of from 2:1 to 1:1.

According to further embodiments of the mixtures and compositions, the weight ratio of the component 1) and the component 2) usually is in the range of from 20,000:1 to 1:10, often in the range of from 10,000:1 to 1:1, regularly in the range of from 5,000:1 to 5:1, preferably in the range of from 5,000:1 to 10:1, more preferably in the range of from 2,000:1 to 30:1, even more preferably in the range of from 2,000:1 to 100:1 and in particular in the range of from 1,000:1 to 100:1.

According to a further embodiments of the binary mixtures and compositions, the weight ratio of the component 1) and the component 2) usually is in the range of from 1:1 to 1:1000, often in the range of from 1:1 to 1:100, regularly in the range of from 1:1 to 1:50, preferably in the range of from 1:1 to 1:20, more preferably in the range of from 1:1 to 1:10, even more preferably in the range of from 1:1 to 1:4 and in particular in the range of from 1:1 to 1:2.

According to further embodiments of the mixtures and compositions, the weight ratio of the component 1) and the component 2) usually is in the range of from 10:1 to 1:20,000, often in the range of from 1:1 to 1:10,000, regularly in the range of from 1:5 to 1:5,000, preferably in the range of from 1:10 to 1:5,000, more preferably in the range of from 1:30 to 1:2,000, even more preferably in the range of from 1:100 to 1:2,000 to and in particular in the range of from 1:100 to 1:1,000.

In the ternary mixtures, i.e. compositions according to the invention comprising the component 1) and component 2) and a compound III (component 3), the weight ratio of component 1) and component 2) depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1, and the weight ratio of component 1) and component 3) usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1.

Any further active components are, if desired, added in a ratio of from 20:1 to 1:20 to the component 1).

These ratios are also suitable for inventive mixtures applied by seed treatment.

The mixtures of active substances can be prepared as compositions comprising besides the active ingredients at least one inert ingredient (auxiliary) by usual means, e.g. by the means given for the compositions of compounds I. Concerning usual ingredients of such compositions reference is made to the explanations given for the compositions containing compounds I.

Examples for composition types are suspensions, capsules, pastes, pastilles, wettable powders or dusts, pressings, granules, insecticidal articles, as well as gel formulations. Herein, it has to be taken into account that each formulation type or choice of auxiliary should not influence the viability of the microorganism during storage of the composition and when finally applied to the soil, plant or plant propagation material. Suitable formulations are e.g. mentioned in WO 2008/002371, U.S. Pat. Nos. 6,955,912, 5,422,107.

Accordingly, the present invention furthermore relates to mixtures comprising one compound of the formula I (component 1, a group represented by the expression “(I)”) and one pesticide II (component 2), wherein pesticide II is an active ingredients selected from the groups A) to O) defined above.

Further embodiments B-1 to B-683 listed in Table B below relate to mixtures comprising as active components one of the in the present specification individualized compounds of the formula I, which is selected from the group of compounds as defined in tables 1 to 90 (component 1, a group represented by the expression “(I)”) and one pesticide II selected from the groups A) to O) as defined herein (component 2, for example, (A.1.1) or azoxystrobin, in embodiment B-1). Further embodiments B-1 to B-683 listed in Table B below relate to the mixtures comprising as active components one of the in the present specification individualized compounds of the formula I, which is selected from the group of compounds I-1 to I-11 as defined in Table I below. Preferably, the compositions described in Table B comprise the active components in synergistically effective amounts.

Table B:

B-1: (I)+(A.1.1), B-2: (I)+(A.1.2), B-3: (I)+(A.1.3), B-4: (I)+(A.1.4), B-5: (I)+(A.1.5), B-6: (I)+(A.1.6), B-7: (I)+(A.1.7), B-8: (I)+(A.1.8), B-9: (I)+(A.1.9), B-10: (I)+(A.1.10), B-11: (I)+(A.1.11), B-12: (I)+(A.1.12), B-13: (I)+(A.1.13), B-14: (I)+(A.1.14), B-15: (I)+(A.1.15), B-16: (I)+(A.1.16), B-17: (I)+(A.1.17), B-18: (I)+(A.1.18), B-19: (I)+(A.1.19), B-20: (I)+(A.1.20), B-21: (I)+(A.1.21), B-22: (I)+(A.1.21a), B-23: (I)+(A.1.22), B-24: (I)+(A.1.25), B-25: (I)+(A.1.34), B-26: (I)+(A.1.35), B-27: (I)+(A.1.36), B-28: (I)+(A.1.37), B-29: (I)+(A.1.38), B-30: (I)+(A.2.), B-3: (A.2.1), B-31: (I)+(A.2.2), B-32: (I)+(A.2.2), B-32: (I)+(A.2.3), B-33: (I)+(A.2.4), B-34: (I)+(A.2.5), B-35: (I)+(A.3.1), B-36: (I)+(A.3.2), B-37: (I)+(A.3.3), B-38: (I)+(A.3.4), B-39: (I)+(A.3.5), B-40: (I)+(A.3.6), B-41: (I)+(A.3.7), B-42: (I)+(A.3.8), B-43: (I)+(A.3.9), B-44: (I)+(A.3.10), B-45: (I)+(A.3.11), B-46: (I)+(A.3.12), B-47: (I)+(A.3.13), B-48: (I)+(A.3.14), B-49: (I)+(A.3.15), B-50: (I)+(A.3.16), B-51: (I)+(A.3.17), B-52: (I)+(A.3.18), B-53: (I)+(A.3.19), B-54: (I)+(A.3.20), B-55: (I)+(A.3.21), B-56: (I)+(A.3.22), B-57: (I)+(A.3.23), B-58: (I)+(A.3.24), B-59: (I)+(A.3.25), B-60: (I)+(A.3.26), B-61: (I)+(A.3.27), B-62: (I)+(A.3.28), B-63: (I)+(A.3.30), B-64: (I)+(A.3.31), B-65: (I)+(A.3.32), B-66: (I)+(A.3.33), B-67: (I)+(A.3.34), B-68: (I)+(A.3.35), B-69: (I)+(A.3.36), B-70: (I)+(A.3.37), B-71: (I)+(A.3.38), B-72: (I)+(A.3.39), B-73: (I)+(A.4.1), B-74: (I)+(A.4.2), B-75: (I)+(A.4.3), B-76: (I)+(A.4.4), B-77: (I)+(A.4.5), B-78: (I)+(A.4.6), B-79: (I)+(A.4.7), B-80: (I)+(A.4.8), B-81: (I)+(A.4.9), B-82: (I)+(A.4.10), B-83: (I)+(A.4.11), B-84: (I)+(A.4.12), B-85: (I)+(B.1.1), B-86: (I)+(B.1.2), B-87: (I)+(B.1.3), B-88: (I)+(B.1.4), B-89: (I)+(B.1.5), B-90: (I)+(B.1.6), B-91: (I)+(B.1.6), B-9I)+(B.1.7), B-92: (I)+(B.1.8), B-93: (I)+(B.1.9), B-94: (I)+(B.1.10), B-95: (I)+(B.1.11), B-96: (I)+(B.1.12), B-97: (I)+(B.1.13), B-98: (I)+(B.1.14), B-99: (I)+(B.1.15), B-100: (I)+(B.1.16), B-101: (I)+(B.1.17), B-102: (I)+(B.1.18), B-103: (I)+(B.1.19), B-104: (I)+(B.1.20), B-105: (I)+(B.1.21), B-106: (I)+(B.1.22), B-107: (I)+(B.1.23), B-108: (I)+(B.1.24), B-109: (I)+(B.1.25), B-110: (I)+(B.1.26), B-111: (I)+(B.1.27), B-112: (I)+(B.1.28), B-113: (I)+(B.1.29), B-114: (I)+(B.1.30), B-115: (I)+(B.1.31), B-116: (I)+(B.1.32), B-117: (I)+(B.1.37), B-118: (I)+(B.1.38), B-119: (I)+(B.1.39), B-120: (I)+(B.1.40), B-121: (I)+(B.1.41), B-122: (I)+(B.1.42), B-123: (I)+(B.1.43), B-124: (I)+(B.1.44), B-125: (I)+(B.1.45), B-126: (I)+(B.1.46), B-127: (I)+(B.1.47), B-128: (I)+(B.1.48), B-129: (I)+(B.1.49), B-130: (I)+(B.1.50), B-131: (I)+(B.1.51), B-132: (I)+(B.1.52), B-133: (I)+(B.2.1), B-134: (I)+(B.2.2), B-135: (I)+(B.2.3), B-136: (I)+(B.2.4), B-137: (I)+(B.2.5), B-138: (I)+(B.2.6), B-139: (I)+(B.2.7), B-140: (I)+(B.2.8), B-141: (I)+(B.3.1), B-142: (I)+(B.4.1), B-143: (I)+(C.1.1), B-144: (I)+(C.1.2), B-145: (I)+(C.1.3), B-146: (I)+(C.1.4), B-147: (I)+(C.1.5), B-148: (I)+(C.1.6), B-149: (I)+(C.1.7), B-150: (I)+(C.2.1), B-151: (I)+(C.2.2), B-152: (I)+(C.2.3), B-153: (I)+(C.2.4), B-154: (I)+(C.2.5), B-155: (I)+(C.2.6), B-156: (I)+(C.2.7), B-157: (I)+(C.2.8), B-158: (I)+(D.1.1), B-159: (I)+(D.1.2), B-160: (I)+(D.1.3), B-161: (I)+(D.1.4), B-162: (I)+(D.1.5), B-163: (I)+(D.1.6), B-164: (I)+(D.1.7), B-165: (I)+(D.1.8), B-166: (I)+(D.1.9), B-167: (I)+(D.1.10), B-168: (I)+(D.1.11), B-169: (I)+(D.1.12), B-170: (I)+(D.1.13), B-171: (I)+(D.1.14), B-172: (I)+(D.1.15), B-173: (I)+(D.1.16), B-174: (I)+(D.2.1), B-175: (I)+(D.2.2), B-176: (I)+(D.2.3), B-177: (I)+(D.2.4), B-178: (I)+(D.2.5), B-179: (I)+(D.2.6), B-180: (I)+(D.2.7), B-181: (I)+(E.1.1), B-182: (I)+(E.1.2), B-183: (I)+(E.1.3), B-184: (I)+(E.2.1), B-185: (I)+(E.2.2), B-186: (I)+(E.2.3), B-187: (I)+(E.2.4), B-188: (I)+(E.2.5), B-189: (I)+(E.2.6), B-190: (I)+(F.1.1), B-191: (I)+(F.1.2), B-192: (I)+(F.1.3), B-193: (I)+(F.1.4), B-194: (I)+(F.1.5), B-195: (I)+(F.2.1), B-196: (I)+(G.1.1), B-197: (I)+(G.1.2), B-198: (I)+(G.1.3), B-199: (I)+(G.1.4), B-200: (I)+(G.2.1), B-201: (I)+(G.2.2), B-202: (I)+(G.2.3), B-203: (I)+(G.2.4), B-204: (I)+(G.2.5), B-205: (I)+(G.2.6), B-206: (I)+(G.2.7), B-207: (I)+(G.3.1), B-208: (I)+(G.3.2), B-209: (I)+(G.3.3), B-210: (I)+(G.3.4), B-211: (I)+(G.3.5), B-212: (I)+(G.3.6), B-213: (I)+(G.3.7), B-214: (I)+(G.4.1), B-215: (I)+(G.5.1), B-216: (I)+(G.5.2), B-217: (I)+(G.5.3), B-218: (I)+(G.5.4), B-219: (I)+(G.5.5), B-220: (I)+(G.5.6), B-221: (I)+(G.5.7), B-222: (I)+(G.5.8), B-223: (I)+(G.5.9), B-224: (I)+(G.5.10), B-225: (I)+(G.5.11), B-226: (I)+(H.1.1), B-227: (I)+(H.1.2), B-228: (I)+(H.1.3), B-229: (I)+(H.1.4), B-230: (I)+(H.1.5), B-231: (I)+(H.1.6), B-232: (I)+(H.1.7), B-233: (I)+(H.2.1), B-234: (I)+(H.2.2), B-235: (I)+(H.2.3), B-236: (I)+(H.2.4), B-237: (I)+(H.2.5), B-238: (I)+(H.2.6), B-239: (I)+(H.2.7), B-240: (I)+(H.2.8), B-241: (I)+(H.2.9), B-242: (I)+(H.3.1), B-243: (I)+(H.3.2), B-244: (I)+(H.3.3), B-245: (I)+(H.3.4), B-246: (I)+(H.3.5), B-247: (I)+(H.3.6), B-248: (I)+(H.3.7), B-249: (I)+(H.3.8), B-250: (I)+(H.3.9), B-251: (I)+(H.3.10), B-252: (I)+(H.3.11), B-253: (I)+(H.4.1), B-254: (I)+(H.4.2), B-255: (I)+(H.4.3), B-256: (I)+(H.4.4), B-257: (I)+(H.4.5), B-258: (I)+(H.4.6), B-259: (I)+(H.4.7), B-260: (I)+(H.4.8), B-261: (I)+(H.4.9), B-262: (I)+(H.4.10), B-263: (I)+(1.1.1), B-264: (I)+(1.1.2), B-265: (I)+(1.2.1), B-266: (I)+(1.2.2), B-267: (I)+(1.2.3), B-268: (I)+(1.2.4), B-269: (I)+(1.2.5), B-270: (I)+(J.1.1), B-271: (I)+(J.1.2), B-272: (I)+(J.1.3), B-273: (I)+(J.1.4), B-274: (I)+(J.1.5), B-275: (I)+(J.1.6), B-276: (I)+(J.1.7), B-277: (I)+(J.1.8), B-278: (I)+(J.1.9), B-279: (I)+(J.1.10), B-280: (I)+(K.1.1), B-281: (I)+(K.1.2), B-282: (I)+(K.1.3), B-283: (I)+(K.1.4), B-284: (I)+(K.1.5), B-285: (I)+(K.1.6), B-286: (I)+(K.1.7), B-287: (I)+(K.1.8), B-288: (I)+(K.1.9), B-289: (I)+(K.1.10), B-290: (I)+(K.1.11), B-291: (I)+(K.1.12), B-292: (I)+(K.1.13), B-293: (I)+(K.1.14), B-294: (I)+(K.1.15), B-295: (I)+(K.1.16), B-296: (I)+(K.1.17), B-297: (I)+(K.1.18), B-298: (I)+(K.1.19), B-299: (I)+(K.1.20), B-300: (I)+(K.1.21), B-301: (I)+(K.1.22), B-302: (I)+(K.1.23), B-303: (I)+(K.1.24), B-304: (I)+(K.1.25), B-305: (I)+(K.1.26), B-306: (I)+(K.1.27), B-307: (I)+(K.1.28), B-308: (I)+(K.1.29), B-309: (I)+(K.1.30), B-310: (I)+(K.1.31), B-311: (I)+(K.1.32), B-312: (I)+(K.1.33), B-313: (I)+(K.1.34), B-314: (I)+(K.1.35), B-315: (I)+(K.1.36), B-316: (I)+(K.1.37), B-317: (I)+(K.1.38), B-318: (I)+(K.1.39), B-319: (I)+(K.1.40), B-320: (I)+(K.1.41), B-321: (I)+(K.1.42), B-322: (I)+(K.1.43), B-323: (I)+(K.1.44), B-324: (I)+(K.1.45), B-325: (I)+(K.1.46), B-326: (I)+(K.1.47), B-327: (I)+(K.1.48), B-328: (I)+(K.1.49), B-329: (I)+(K.1.50), B-330: (I)+(K.1.51), B-331: (I)+(K.1.52), B-332: (I)+(K.1.53), B-333: (1)+(K.1.54), B-334: (I)+(O.1.1), B-335: (I)+(O.1.2), B-336: (I)+(O.1.3), B-337: (I)+(O.1.4), B-338: (1)+(O.1.5), B-339: (I)+(O.1.6), B-340: (I)+(O.1.7), B-341: (I)+(O.1.8), B-342: (I)+(O.1.9), B-343: (1)+(O.1.10), B-344: (I)+(O.1.11), B-345: (I)+(O.1.12), B-346: (I)+(O.1.13), B-347: (I)+(O.1.14), B-348: (I)+(O.1.15), B-349: (I)+(O.1.16), B-350: (I)+(O.1.17), B-351: (I)+(O.1.18), B-352: (I)+(O.1.19), B-353: (I)+(O.1.20), B-354: (I)+(O.1.21), B-355: (I)+(O.1.22), B-356: (I)+(O.1.23), B-357: (I)+(O.1.24), B-358: (I)+(O.1.25), B-359: (I)+(O.1.26), B-360: (I)+(O.1.27), B-361: (I)+(O.1.28), B-362: (I)+(O.1.29), B-363: (I)+(O.1.30), B-364: (I)+(O.1.31), B-365: (I)+(O.1.32), B-366: (I)+(O.1.33), B-367: (I)+(O.1.34), B-368: (I)+(O.1.35), B-369: (I)+(O.1.36), B-370: (I)+(O.1.37), B-371: (I)+(O.1.38), B-372: (I)+(O.1.39), B-373: (I)+(O.1.40), B-374: (I)+(O.1.41), B-375: (I)+(O.1.42), B-376: (I)+(O.1.43), B-377: (I)+(O.1.44), B-378: (I)+(O.1.45), B-379: (I)+(O.1.46), B-380: (I)+(O.1.47), B-381: (I)+(O.1.48), B-382: (I)+(O.1.49), B-383: (I)+(O.1.50), B-384: (I)+(O.1.51), B-385: (I)+(O.1.52), B-386: (I)+(O.1.53), B-387: (I)+(O.1.54), B-388: (I)+(O.1.55), B-389: (I)+(O.1.56), B-390: (I)+(O.1.57), B-391: (I)+(O.1.58), B-392: (I)+(O.1.59), B-393: (I)+(O.1.60), B-394: (I)+(O.1.61), B-395: (I)+(O.1.62), B-396: (I)+(O.1.63), B-397: (I)+(O.1.64), B-398: (I)+(O.1.65), B-399: (I)+(O.1.66), B-400: (I)+(O.1.67), B-401: (I)+(O.1.68), B-402: (I)+(O.1.69), B-403: (I)+(O.1.70), B-404: (I)+(O.1.71), B-405: (I)+(O.1.72), B-406: (I)+(O.1.73), B-407: (I)+(O.1.74), B-408: (I)+(O.1.75), B-409: (I)+(O.1.76), B-410: (I)+(O.1.77), B-411: (I)+(O.1.78), B-412: (I)+(O.1.79), B-413: (I)+(O.1.80), B-414: (I)+(O.1.81), B-415: (I)+(O.1.82), B-416: (I)+(O.1.83), B-417: (I)+(O.1.84), B-418: (I)+(O.1.85), B-419: (I)+(O.1.86), B-420: (I)+(O.1.87), B-421: (I)+(O.1.88), B-422: (I)+(O.1.89), B-423: (I)+(O.1.90), B-424: (I)+(O.1.91), B-425: (I)+(O.2.1), B-426: (I)+(O.2.2), B-427: (I)+(O.2.3), B-428: (I)+(O.2.4), B-429: (I)+(O.2.5), B-430: (I)+(O.2.6), B-431: (I)+(O.2.7), B-432: (I)+(O.3.1), B-433: (I)+(O.3.2), B-434: (I)+(O.3.3), B-435: (I)+(O.3.4), B-436: (I)+(O.3.5), B-437: (I)+(O.3.6), B-438: (I)+(O.3.7), B-439: (I)+(O.3.8), B-440: (I)+(O.3.9), B-441: (I)+(O.3.10), B-442: (I)+(O.3.11), B-443: (I)+(O.3.12), B-444: (I)+(O.3.13), B-445: (I)+(O.3.14), B-446: (I)+(O.3.15), B-447: (I)+(O.3.16), B-448: (I)+(O.3.17), B-449: (I)+(O.3.18), B-450: (I)+(O.3.19), B-451: (I)+(O.3.20), B-452: (I)+(O.3.21), B-453: (I)+(O.3.22), B-454: (I)+(O.3.23), B-455: (I)+(O.3.24), B-456: (I)+(O.3.25), B-457: (I)+(O.3.26), B-458: (I)+(O.3.27), B-459: (I)+(O.3.28), B-460: (I)+(O.3.29), B-461: (I)+(O.3.30), B-462: (I)+(O.3.31), B-463: (I)+(O.3.32), B-464: (I)+(O.3.33), B-465: (I)+(O.3.34), B-466: (I)+(O.3.35), B-467: (I)+(O.3.36), B-468: (I)+(O.3.37), B-469: (I)+(O.3.38), B-470: (I)+(O.3.39), B-471: (I)+(O.3.40), B-472: (I)+(O.3.41), B-473: (I)+(O.3.42), B-474: (I)+(O.3.43), B-475: (I)+(O.3.44), B-476: (I)+(O.3.45), B-477: (I)+(O.3.46), B-478: (I)+(O.3.47), B-479: (I)+(O.3.48), B-480: (I)+(O.3.49), B-481: (I)+(O.3.50), B-482: (I)+(O.3.51), B-483: (I)+(O.3.52), B-484: (I)+(O.4.1), B-485: (I)+(O.4.2), B-486: (I)+(O.4.3), B-487: (I)+(O.4.4), B-488: (I)+(O.4.5), B-489: (I)+(O.4.6), B-490: (I)+(O.4.7), B-491: (I)+(O.4.8), B-492: (I)+(O.4.9), B-493: (I)+(O.4.10), B-494: (I)+(O.4.11), B-495: (I)+(O.4.12), B-496: (I)+(O.4.13), B-497: (I)+(O.4.14), B-498: (I)+(O.4.15), B-499: (I)+(O.5.1), B-500: (I)+(O.5.2), B-501: (I)+(O.6.1), B-502: (I)+(O.6.2), B-503: (I)+(O.6.3), B-504: (I)+(O.6.4), B-505: (I)+(O.6.5), B-506: (I)+(O.7.1), B-507: (I)+(O.7.2), B-508: (I)+(O.7.3), B-509: (I)+(O.7.4), B-510: (I)+(O.7.5), B-511: (I)+(O.8.1), B-512: (I)+(O.8.2), B-513: (I)+(O.8.3), B-514: (I)+(O.8.4), B-515: (I)+(O.8.5), B-516: (I)+(O.9.1), B-517: (I)+(O.9.2), B-518: (I)+(O.9.3), B-519: (I)+(O.10.1), B-520: (I)+(O.10.2), B-521: (I)+(O.10.3), B-522: (I)+(O.10.4), B-523: (I)+(O.11.1), B-524: (I)+(O.11.2), B-525: (I)+(O.11.3), B-526: (I)+(O.11.4), B-527: (I)+(O.11.5), B-528: (I)+(O.11.6), B-529: (I)+(O.11.7), B-530: (I)+(O.11.8), B-531: (I)+(O.11.9), B-532: (I)+(O.11.10), B-533: (I)+(O.11.11), B-534: (I)+(O.11.12), B-535: (I)+(O.11.13), B-536: (I)+(O.12.1), B-537: (I)+(O.12.2), B-538: (I)+(O.12.3), B-539: (I)+(O.12.4), B-540: (I)+(O.12.5), B-541: (I)+(O.12.6), B-542: (I)+(O.13.1), B-543: (I)+(O.13.2), B-544: (I)+(O.13.3), B-545: (I)+(O.14.1), B-546: (I)+(O.14.2), B-547: (I)+(O.14.3), B-548: (I)+(O.14.4), B-549: (I)+(O.15.1), B-550: (I)+(O.15.2), B-551: (I)+(O.15.3), B-552: (I)+(O.15.4), B-553: (I)+(O.15.5), B-554: (I)+(O.15.6), B-555: (I)+(O.15.7), B-556: (I)+(O.15.8), B-557: (I)+(O.15.9), B-558: (I)+(O.15.10), B-559: (I)+(O.15.11), B-560: (I)+(O.16.1), B-561: (I)+(O.17.1), B-562: (I)+(O.18.1), B-563: (I)+(O.18.2), B-564: (I)+(O.18.3), B-565: (I)+(O.18.4), B-566: (I)+(O.18.5), B-567: (I)+(O.19.1), B-568: (I)+(O.20.1), B-569: (I)+(O.20.2), B-570: (I)+(O.20.3), B-571: (I)+(O.20.4), B-572: (I)+(O.21.1), B-573: (I)+(O.21.2), B-574: (I)+(O.21.3), B-575: (I)+(O.21.4), B-576: (I)+(O.21.5), B-577: (I)+(O.21.6), B-578: (I)+(O.21.7), B-579: (I)+(O.22.1), B-580: (I)+(O.22.2), B-581: (I)+(O.22.3), B-582: (I)+(O.22.4), B-583: (I)+(O.23.1), B-584: (I)+(O.23.2), B-585: (I)+(O.23.3), B-586: (I)+(O.23.4), B-587: (I)+(O.24.1), B-588: (I)+(O.24.2), B-589: (I)+(O.24.3), B-590: (I)+(O.24.4), B-591: (I)+(O.24.5), B-592: (I)+(O.25.1), B-593: (I)+(O.25.2), B-594: (I)+(O.26.1), B-595: (I)+(O.26.2), B-596: (I)+(O.26.3), B-597: (I)+(O.26.4), B-598: (I)+(O.26.5), B-599: (I)+(O.26.6), B-600: (I)+(O.26.7), B-601: (I)+(O.26.8), B-602: (I)+(O.26.9), B-603: (I)+(O.26.10), B-604: (I)+(O.26.11), B-605: (I)+(O.26.12), B-606: (I)+(O.26.13), B-607: (I)+(O.26.14), B-608: (I)+(O.26.15), B-609: (I)+(O.26.16), B-610: (I)+(O.26.17), B-611: (I)+(O.26.18), B-612: (I)+(O.27.1), B-613: (I)+(O.28.1), B-614: (I)+(O.28.2), B-615: (I)+(O.28.3), B-616: (I)+(O.28.4), B-617: (I)+(O.28.5), B-618: (I)+(O.28.7), B-619: (I)+(O.28.8), B-620: (I)+(O.28.9), B-621: (I)+(O.28.10), B-622: (I)+(O.28.11), B-623: (I)+(O.28.12), B-624: (I)+(O.28.13), B-625: (I)+(O.28.14), B-626: (I)+(O.28.15), B-627: (I)+(O.28.16), B-628: (I)+(O.28.17), B-629: (I)+(O.28.18), B-630: (I)+(O.28.19), B-631: (I)+(O.28.20), B-632: (I)+(O.28.21), B-633: (I)+(O.28.22), B-634: (I)+(O.28.23), B-635: (I)+(O.28.24), B-636: (I)+(O.28.25), B-637: (I)+(O.28.26), B-638: (I)+(O.28.27), B-639: (I)+(O.28.28), B-640: (I)+(O.28.29), B-641: (I)+(O.28.30), B-642: (I)+(O.28.31), B-643: (I)+(O.28.42), B-644: (I)+(O.28.43), B-645: (I)+(O.28.44), B-646: (I)+(O.28.45), B-647: (I)+(O.28.46), B-648: (I)+(O.28.47), B-649: (I)+(O.28.48), B-650: (I)+(O.28.49), B-651: (I)+(O.28.50), B-652: (I)+(O.28.51), B-653: (I)+(O.28.52), B-654: (I)+(O.28.53), B-655: (I)+(O.28.54), B-656: (I)+(O.28.55), B-657: (I)+(O.28.56), B-658: (I)+(O.28.57), B-659: (I)+(O.28.58), B-660: (I)+(O.28.59), B-661: (I)+(O.28.60), B-662: (I)+(O.28.61), B-663: (I)+(O.28.62), B-664: (I)+(O.28.63), B-665: (I)+(O.28.64), B-666: (I)+(O.28.65), B-667: (I)+(O.28.66), B-668: (I)+(O.28.67), B-669: (I)+(O.28.68), B-670: (I)+(O.28.69), B-671: (I)+(O.28.70), B-672: (I)+(O.28.71), B-673: (I)+(O.28.72), B-674: (I)+(O.28.73), B-675: (I)+(O.28.74), B-676: (I)+(O.28.75), B-677: (I)+(O.28.76), B-678: (I)+(O.28.77), B-679: (I)+(O.28.78), B-680: (I)+(O.28.79), B-681: (I)+(O.28.80), B-682: (I)+(O.28.81), B-683: (I)+(O.28.82).

### I. SYNTHESIS EXAMPLES

### Example 1: Preparation of 2,2-difluoro-N-methyl-2-[4-[5-(trifluoromethyl)-1,2,4-oxadiazol-3-yl]phenoxy]acetamide (I-1)

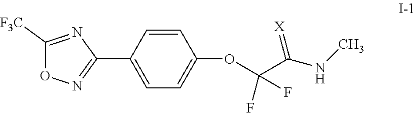

### I.1) Ethyl 2-(4-cyanophenoxy)-2,2-difluoro-acetate

To the solution of 4-hydroxybenzonitrile (1 g, 8.4 mmol) in DMF (30 mL) was added 1,8-di-azabicyclo[5.4.0]undec-7-ene (DBU) (2.6 g, 16.8 mmol) and ethyl 2-bromo-2,2-difluoro-acetate (3.4 g, 16.8 mmol) at 25° C. and stirring was continued for 16 h. The reaction mixture was diluted with brine (50 mL) and extracted with MTBE. The organic layer was successively washed with a saturated aqueous solution of sodium chloride, dried over sodium sulfate and freed from solvent under reduced pressure. Flash chromatography on silica gel using cyclohexane-ethyl acetate provided the titled product (1.65 g) as colorless oil.

1H NMR (CDCl3): δ [ppm]=1.39 (t, J=7.15 Hz, 3H) 4.42 (q, J=7.15 Hz, 2H) 7.34 (d, J=8.78 Hz, 2H) 7.66-7.77 (m, 2H).

### I.2) 2-(4-cyanophenoxy)-2,2-difluoro-N-methyl-acetamide

The solution of methylamine in THF (6 mL, 12 mmol) in dichloromethane (DCM) (30 mL) was added trimethylaluminium (6 mL, 12 mmol) dropwise, the mixture was stirred for 1 h, then ethyl 2-(4-cyanophenoxy)-2,2-difluoro-acetate (1.45 g, 6 mmol) in DCM was added dropwise and the mixture was stirred for 16 h at 25° C. The reaction mixture was quenched with water (100 mL), extracted with DCM (50 mL) and ethyl acetate (EtOAc, 100 mL). The organic layer was successively washed with a saturated aqueous solution of sodium chloride, dried over sodium sulfate and freed from solvent under reduced pressure to provide the titled product (1.65 g) as a yellow solid. 1H NMR (CDCl3): δ [ppm]=2.91 (d, J=5.02 Hz, 3H) 6.46 (br d, J=12.30 Hz, 1H) 7.27 (d, J=8.78 Hz, 2H) 7.58-7.67 (m, 2H).

### I.3) 2,2-Difluoro-2-[4-[(Z)—N′-hydroxycarbamimidoyl]phenoxy]-N-methyl-acetamide

2-(4-Cyanophenoxy)-2,2-difluoro-N-methyl-acetamide (1.25 g, 0.006 mol), hydroxylamine hydrochloride (1.2 g, 0.017 mol) and triethylamine (1.72 g, 0.017 mol) were dissolved in ethanol (50 mL). The reaction mixture was heated at 70° C. for 16 h. The solvent was removed to give 5.68 g of a white solid. The crude material was used without further purification.

1H NMR (CDCl3): δ [ppm]=2.85 (s, 3H) 7.19 (d, J=8.66 Hz, 2H) 7.58 (d, J=8.78 Hz, 2H).

### I.4) 2,2-Difluoro-N-methyl-2-[4-[5-(trifluoromethyl)-1,2,4-oxadiazol-3-yl]phenoxy]acetamide

2,2-Difluoro-2-[4-[(Z)—N′-hydroxycarbamimidoyl]phenoxy]-N-methyl-acetamide (5.68 g, 0.022 mol) was dissolved in dimethylacetamide (100 ml). Trifluoroacetic acid anhydride (TFAA) was added (9.25 g, 0.044 mol) dropwise at 0° C. and the mixture was stirred for 1 h at 25° C. The reaction mixture was diluted with brine (100 mL) and extracted with EtOAc (50 mL). The organic layer was successively washed with a saturated aqueous solution of sodium chloride, dried over sodium sulfate and freed from solvent. The residue was purified by prep-HPLC (gradient elution, water/ACN both containing 0.1% TFA) to give 1.4 g of the desired product as a white solid.

1H NMR (CDCl₃): δ [ppm]=3.00 (d, J=5.02 Hz, 3H) 6.53 (br s, 1H) 7.39 (d, J=8.66 Hz, 2H) 8.11-8.21 (m, 2H).

With due modification of the starting compounds, the procedures shown in the synthesis examples below were used to obtain further compounds I. The resulting compounds, together with physical data, are listed in Table I below.

TABLE I
Compounds I-1 to I-19 of formula I.1 with physical
data, wherein the meaning of R¹, R³, R⁴,
X and W are as defined in each line.

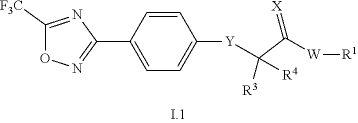

| Ex. | | | | | HPLC Rₜ | m.p. [°C]; |
|---|---|---|---|---|---|---|
| no | R³, R⁴ | X | W | R¹ | (min)* | ¹H-NMR (δ) |
| I-1 | F, F | O | NH | —CH₃ | 1.17 | 110; δ [ppm] = 3.00 (d, |
| | | | | | | J = 5.02 Hz, 3H) 6.53 (br s, |
| | | | | | | 1H) 7.39 (d, J = 8.66 Hz, |
| | | | | | | 2H) 8.11-8.21 (m, 2 H) |
| I-2 | F, F | O | NCH₃ | —CH₃ | 1.25 | 54; δ [ppm] = 0.6-0.9 (m, |
| | | | | | | 4H); 3.2 (s, 3H); 7.3 (m, |
| | | | | | | 2H); 8.3 (m, 2H) |
| I-3 | F, F | O | NCH₃ | cyclopropyl | 1.33 | 47 |
| I-4 | F, F | O | NH | cyclopropyl | 1.21 | 108; 5 [ppm] = 0.6-0.9 |
| | | | | | | (m, 4H); 2.85 (m, 1H); 7.4 |
| | | | | | | (m, 2H); 8.2 (m, 2H) |
| I-5 | F, F | O | NH | isopropyl | 1.24 | 104 |
| I-6 | F, F | O | NH | cycloheptyl | 1.31 | 120 |
| I-7 | F, F | O | NCH₃ | —CH₂CH₃ | 1.28 | — |
| I-8 | F, F | O | NH | cyclobutyl | 1.27 | 114 |
| I-9 | F, F | O | NH | —C(CH₃)₃ | 1.28 | 64 |
| I-10 | H, H | O | NCH₃ | —CH₃ | 1.05 | — |
| I-11 | H, H | O | NH | —CH₃ | 1.08 | — |
| I-12 | CH₃, CH₃ | O | NH | —CH₃ | 1.17 | — |
| I-13 | CH₃, CH₃ | O | NCH₃ | —CH₃ | 1.28 | — |
| I-14 | H, CH₃ | O | NH | —CH₃ | 1.10 | — |
| I-15 | H, CH₃ | O | NCH₃ | —CH₃ | 1.13 | — |
| I-16 | F, F | O | NH | —CH₂CF₃ | 1.27 | 101 |
| I-17 | F, F | S | NH | cyclopropyl | 1.33 | — |
| I-18 | F, F | S | NH | —CH₃ | 1.28 | 115 |
| I-19 | Br, CF₃ | O | NCH₃ | —CH₃ | 1.38 | — |

Legend of Table I:
m.p. = melting point; Rₜ = HPLC Retention time.
*HPLC: High Performance Liquid Chromatography; HPLC-column Kinetex XB C18 1,7 μ (50 x 2,1 mm); eluent: acetonitrile/water + 0.1% trifluoroacetic acid (gradient from 5:95 to 100:0 in 1.5 min at 60° C, flow gradient from 0.8 to 1.0 ml/min in 1.5 min). MS: Quadrupol Electrospray Ionisation, 80 V (positive mode).

### II. BIOLOGICAL EXAMPLES FOR FUNGICIDAL ACTIVITY

Glass House Trials

The fungicidal action of the compounds of formula I was demonstrated by the following experiments: spray solutions were prepared in several steps. A mixture was prepared of acetone and/or dimethylsulfoxide and the wetting agent/emulsifier Wettol, which is based on ethoxylated alkylphenoles, in a relation (volume) solvent-emulsifier of 99 to 1 was added to the compound to give a total of 5 ml. Water was then added to a total volume of 100 ml. This stock solution was diluted with the described solvent-emulsifier-water mixture to the given concentration.

II.1) Curative Control of Soy Bean Rust on Soy Beans Caused by Phakopsora pachyrhizi

Leaves of pot-grown soy bean seedlings were inoculated with spores of Phakopsora pachyrhizi To ensure the success of the artificial inoculation, the plants were transferred to a humid chamber with a relative humidity of about 95% and 20 to 24° C. for 24 hours. The next day the plants were cultivated for 3 days in a greenhouse chamber at 23 to 27° C. and a relative humidity between 60 and 80%. Then the plants were sprayed to run-off with an aqueous suspension, containing the concentration of active ingredient or their mixture as described below. The plants were allowed to air-dry. Then the trial plants were cultivated for 14 days in a greenhouse chamber at 23 to 27° C. and a relative humidity between 60 and 80%. The extent of fungal attack on the leaves was visually assessed as % diseased leaf area.

In this test, the plants which had been treated with 63 ppm of the active compound I-1, I-4, I-5, I-8, I-10, I-11, I-12, I-13, I-14, I-16 and 1-18 showed a diseased leaf area of at most 12%, whereas the untreated plants showed 90% diseased leaf area.

### II.2) Protective Control of Soy Bean Rust on Soy Beans Caused by Phakopsora pachyrhizi

Leaves of pot-grown soy bean seedlings were sprayed to run-off with an aqueous suspension, containing the concentration of active ingredient or their mixture as described below. The plants were allowed to air-dry. The trial plants were cultivated for 2 days in a greenhouse chamber at 23 to 27° C. and a relative humidity between 60 and 80%. Then the plants were inoculated with spores of Phakopsora pachyrhizi To ensure the success the artificial inoculation, the plants were transferred to a humid chamber with a relative humidity of about 95% and 20 to 24° C. for 24 hours. The trial plants were cultivated for fourteen days in a greenhouse chamber at 23 to 27° C. and a relative humidity between 60 and 80%. The extent of fungal attack on the leaves was visually assessed as % diseased leaf area.

In this test, the plants which had been treated with 63 ppm of the active compound I-1, I-2, I-3, I-4, I-5, I-6, I-7, I-8, I-9, I-10, I-11, I-12, I-13, I-14, I-15, I-16, I-17, I-18 and 1-19 showed a diseased leaf area of at most 2%, whereas the untreated plants showed 90% diseased leaf area.

### II.3) Curative Control of Brown Rust on Wheat Caused by Puccinia recondita

The first two developed leaves of pot-grown wheat seedling were dusted with spores of Puccinia recondita. To ensure the success the artificial inoculation, the plants were transferred to a humid chamber without light and a relative humidity of 95 to 99% and 20 to 24° C. for 24 hours. The next day the plants were cultivated for 3 days in a greenhouse chamber at 20 to 24° C. and a relative humidity between 65 and 70%. Then the plants were sprayed to run-off with an aqueous suspension, containing the concentration of active ingredient or their mixture as described below. The plants could air-dry. Then the trial plants were cultivated for 8 days in a greenhouse chamber at 20 to 24° C. and a relative humidity between 65 and 70%. The extent of fungal attack on the leaves was visually assessed as % diseased leaf area.

In this test, the plants which had been treated with 63 ppm of the active compound 1-2 showed a diseased leaf area of 3%, whereas the untreated plants showed 80% diseased leaf area.

In this test, the plants which had been treated with 63 ppm of the active compound I-1, I-4, I-5, I-10 and 1-14 showed a diseased leaf area of at most 9%, whereas the untreated plants showed 90% diseased leaf area.

### II.4) Preventative Control of Brown Rust on Wheat Caused by Puccinia recondita

The first two developed leaves of pot-grown wheat seedling were sprayed to run-off with an aqueous suspension, containing the concentration of active ingredient or their mixture as described below. The next day the plants were inoculated with spores of Puccinia recondita. To ensure the success the artificial inoculation, the plants were transferred to a humid chamber without light and a relative humidity of 95 to 99% and 20 to 24° C. for 24 hours. Then the trial plants were cultivated for 6 days in a greenhouse chamber at 20 to 24° C. and a relative humidity between 65 and 70%. The extent of fungal attack on the leaves was visually assessed as % diseased leaf area.

In this test, the plants which had been treated with 63 ppm of the active compound 1-2 showed a diseased leaf area of 2%, whereas the untreated plants showed 80% diseased leaf area.

In this test, the plants which had been treated with 63 ppm of the active compound I-1, I-3, I-4, I-10, I-11, I-12, I-14 and I-17 showed a diseased leaf area of at most 13%, whereas the untreated plants showed 90% diseased leaf area.

1.-15. (canceled)

16. A compound of the formula I 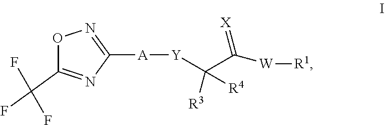
wherein:
A is phenyl or a 5- or 6-membered aromatic heterocycle; wherein the ring member atoms of the aromatic heterocycle include besides carbon atoms 1, 2, 3 or 4 heteroatoms selected from the group consisting of N, O and S as ring member atoms; and wherein the phenyl ring or the 5- or 6-membered aromatic heterocycle are unsubstituted or substituted with 1, 2, 3 or 4 identical or different groups R^{A}; wherein
R^{A} is halogen, cyano, diC₁-C₆-alkylamino, C₁-C₆-alkyl, C₁-C₆-alkoxy, C₁-C₆-alkylthio, C₁-C₆-alkylsulfinyl, C₁-C₆-alkylsulfonyl, C₂-C₆-alkenyl, C₂-C₆-alkynyl, C₃-C₁₁-cycloalkyl or C₃-C₁₁-cycloalkoxy; and wherein any of the aliphatic or cyclic moieties are unsubstituted or substituted with 1, 2, 3 or 4 identical or different groups R^{a}; wherein
R^{a} is halogen, cyano, C₁-C₆-alkyl, C₁-C₆-haloalkyl, C₁-C₆-alkoxy, or C₁-C₆-haloalkoxy;
Y is O or S;
R³, R⁴ independently of each other are selected from the group consisting of hydrogen, halogen, cyano, C₁-C₄-alkyl, C₁-C₄-alkenyl, C₁-C₄-alkynyl, C₁-C₄-alkoxy C₁-C₄-haloalkyl and C₁-C₄-haloalkoxy; or
R³ and R⁴ together with the carbon atom to which they are bound form a saturated 3- to 7-membered carbocycle or a saturated 3- to 6-membered heterocycle; wherein the saturated heterocycle includes beside carbon atoms 1, 2 or 3 heteroatoms independently selected from the group consisting of N, O and S as ring member atoms; and wherein said N ring member atom is substituted with the group R^{N};
wherein
R^{N} is hydrogen, C₁-C₆-alkyl or halogen;
and wherein said S ring member atom is unsubstituted or substituted with 1 or 2 oxo radicals; and wherein one or two of the ring member —CH₂— groups of said saturated carbocycle or of said saturated heterocycle may be replaced by one or two groups independently selected from the group consisting of —C(═O)— and —C(═S)—;
and wherein the carbon ring member atoms of the saturated carbocycle or of the saturated heterocycle are unsubstituted or substituted with a total number of 1, 2, 3, 4 or up to the maximum possible number of identical or different radicals selected from the group consisting of halogen, cyano, C₁-C₆-alkyl, C₁-C₆-alkoxy and C₃-C₁₁-cycloalkyl;
X is O or S;
W is NR², O or S;
R² is selected from the group consisting of hydrogen, CH(═O), C₁-C₆-alkyl, C₂-C₆-alkenyl, C₂-C₆-alkynyl, C₁-C₆-alkoxy, C₃-C₁₁-cycloalkyl, C₃-C₈-cycloalkenyl, C₃-C₁₁-cycloalkyl-C₁-C₄-alkyl, phenyl-C₁-C₄-alkyl, phenyl, pyridinyl, —C(═O)—(C₁-C₆-alkyl), —C(═O)—(C₃-C₁₁-cycloalkyl),
—C(═O)—(C₁-C₆-alkoxy) and —N(R^{2a})₂; wherein
R^{2a} is independently selected from the group consisting of hydrogen, OH, C₁-C₆-alkyl, C₂-C₆-alkenyl, C₂-C₆-alkynyl, C₃-C₁₁-cycloalkyl, C₃-C₈-cycloalkenyl, C₁-C₆-alkoxy, C₁-C₄-alkoxy-C₁-C₄-alkyl and C₁-C₆-alkylthio;
and wherein any of the aliphatic or cyclic groups in R² are unsubstituted or substituted with 1, 2, 3 or up to the maximum possible number of identical or different radicals selected from the group consisting of halogen, hydroxy, oxo, cyano, C₁-C₆-alkyl, C₁-C₆-alkoxy and C₃-C₁₁-cycloalkyl;
R¹ is hydrogen, CH(O), C₁-C₆-alkyl, C₁-C₆-alkoxy, C₃-C₁₁-cycloalkyl, C₃-C₈-cycloalkenyl, C₂-C₆-alkenyl, C₂-C₆-alkynyl, C₁-C₆-alkoxyimino-C₁-C₄-alkyl, C₂-C₆-alkenyloxyimino-C₁-C₄-alkyl, C₂-C₆-alkynyloxyimino-C₁-C₄-alkyl, phenyl-C₁-C₄-alkyl, phenyl-C₁-C₄-alkenyl, phenyl-C₁-C₄-alkynyl, heteroaryl-C₁-C₄-alkyl, phenyl, naphthyl or a 3- to 10-membered saturated, partially unsaturated or aromatic mono- or bicyclic heterocycle, wherein the ring member atoms of said mono- or bicyclic heterocycle include besides carbon atoms further 1, 2, 3 or 4 heteroatoms selected from the group consisting of N, O and S as ring member atoms; and wherein the heteroaryl group in the group heteroaryl-C₁-C₄-alkyl is a 5- or 6-membered aromatic heterocycle, wherein the ring member atoms of the heterocyclic ring include besides carbon atoms 1, 2, 3 or 4 heteroatoms selected from N, O and S as ring member atoms; and wherein any of the above-mentioned aliphatic or cyclic groups are unsubstituted or substituted with 1, 2, 3 or up to the maximum possible number of identical or different groups R^{1a}; or
if W is NR², R¹ and R², together with the nitrogen atom to which R² is attached, may form a saturated or partially unsaturated mono- or bicyclic 3- to 10-membered heterocycle, wherein the heterocycle includes beside one nitrogen atom and one or more carbon atoms no further heteroatoms or 1, 2 or 3 further heteroatoms independently selected from the group consisting of N, O and S as ring member atoms; and wherein the heterocycle is unsubstituted or substituted with 1, 2, 3, 4 or up to the maximum possible number of identical or different groups R^{1a}; or
if W is NR² and R² is —N(R^{2a})₂, R¹ and one of the two groups R^{2a}, together with the nitrogen atom to which said R^{2a} is attached, and together with the nitrogen atom to which the group R¹ attached, may form a saturated or partially unsaturated mono- or bicyclic 3- to 10-membered heterocycle, wherein the heterocycle includes beside two nitrogen atoms and one or more carbon atoms no further heteroatoms or 1, 2 or 3 further heteroatoms independently selected from the group consisting of N, O and S as ring member atoms; and wherein the heterocycle is unsubstituted or substituted with 1, 2, 3, 4 or up to the maximum possible number of identical or different groups R^{1a};
R^{1a} is halogen, oxo, cyano, NO₂, OH, SH, NH₂, C₁-C₆-alkyl, C₂-C₆-alkenyl, C₂-C₆-alkynyl, C₁-C₆-haloalkyl, C₁-C₆-alkoxy, C₁-C₆-haloalkoxy, C₁-C₆-alkylthio, C₁-C₆-haloalkylthio, C₃-C₁₁-cycloalkyl, —NHSO₂—C₁-C₄-alkyl, —(C═O)—C₁-C₄-alkyl, —C(═O)—C₁-C₄-alkoxy, C₁-C₆-alkylsulfonyl, hydroxyC₁-C₄-alkyl, —C(═O)—NH₂, —C(═O)—NH(C₁-C₄-alkyl), C₁-C₄-alkylthio-C₁-C₄-alkyl, aminoC₁-C₄-alkyl, C₁-C₄-alkylamino-C₁-C₄-alkyl, diC₁-C₄-alkylamino-C₁-C₄-alkyl, aminocarbonyl-C₁-C₄-alkyl or C₁-C₄-alkoxy-C₁-C₄-alkyl;
or the N-oxides, or the agriculturally acceptable salts thereof.

17. The compound of claim 16, wherein A is a phenyl ring.

18. The compound of claim 16, wherein A is a thiophene ring.

19. The compound of claim 16, wherein Y is O.

20. The compound of claim 16, wherein X is O.

21. The compound of claim 16, wherein R³ and R⁴ independently of each other are selected from the group consisting of hydrogen, fluorine, methyl, trifluoromethyl and OCHF₂, or R³ and R⁴ together with the carbon atom to which they are bound form a cyclopropyl ring.

22. The compound of claim 21, wherein R³ and R⁴ are both halogen.

23. The compound of claim 16, wherein W is NR².

24. The compound of claim 16, wherein R² is hydrogen, CH(═O), C₁-C₆-alkyl, C₁-C₆-alkoxy, C₂-C₆-alkenyl, propargyl, C₃-C₈-cycloalkyl, C₃-C₈-cycloalkenyl, C₃-C₈-cycloalkyl-C₁-C₄-alkyl, phenyl, C₁-C₆-alkylamino or diC₁-C₆-alkylamino; and wherein any of the aliphatic or cyclic groups are unsubstituted or substituted with 1, 2, 3, 4 or up to the maximum possible number of identical or different radicals selected from the group consisting of halogen, cyano, C₁-C₆-alkyl and C₁-C₆-alkoxy.

25. The compound of claim 16, wherein R¹ is hydrogen, C₁-C₆-alkyl, C₁-C₆-alkoxy, C₃-C₈-cycloalkyl, phenyl or benzyl, and wherein any of the aliphatic or cyclic groups are substituted with 1, 2 or 3 of identical or different groups R^{1a}; wherein R^{1a} is selected from fluorine, chlorine, cyano, methyl, ethyl, methoxy, trifluoromethyl, trifluoromethoxy, difluoromethyl, difluoromethoxy and cyclopropyl.

26. Intermediate compounds of the formulae IV or V, 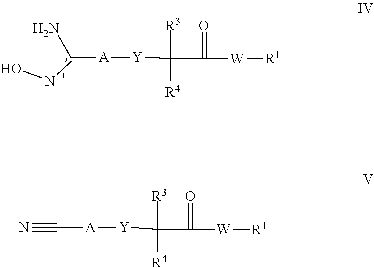
wherein the variables A, Y, W, R¹, R³ and R⁴ are as defined in claim 16 for compounds of the formula I.

27. An agrochemical composition, which comprises an auxiliary and at least one compound of the formula I of claim 16 or an N-oxide or an agriculturally acceptable salt thereof.

28. An agrochemical composition of claim 27 further comprising seed, wherein the amount of the compound of the formula I, or an N-oxide, or an agriculturally acceptable salt thereof, is from 0.1 g to 10 kg per 100 kg of seed.

29. A method for combating phytopathogenic harmful fungi, which process comprises treating the fungi, the plants, the soil or seeds to be protected against fungal attack, with an effective amount of at least one compound of formula I of claim 16 or an N-oxide or an agriculturally acceptable salt thereof.

30. A method for combating phytopathogenic harmful fungi, which process comprises treating the fungi, the plants, the soil or seeds to be protected against fungal attack, with an effective amount of the agrochemical composition as defined in claim 27.

31. The compound of claim 17, wherein Y is O.

32. The compound of claim 17, wherein X is O.

33. The compound of claim 17, wherein R³ and R⁴ independently of each other are selected from the group consisting of hydrogen, fluorine, methyl, trifluoromethyl and OCHF₂, or R³ and R⁴ together with the carbon atom to which they are bound form a cyclopropyl ring.

34. The compound of claim 17, wherein W is NR².

35. The compound of claim 17, wherein R² is hydrogen, CH(═O), C₁-C₆-alkyl, C₁-C₆-alkoxy, C₂-C₆-alkenyl, propargyl, C₃-C₈-cycloalkyl, C₃-C₈-cycloalkenyl, C₃-C₈-cycloalkyl-C₁-C₄-alkyl, phenyl, C₁-C₆-alkylamino or diC₁-C₆-alkylamino; and wherein any of the aliphatic or cyclic groups are unsubstituted or substituted with 1, 2, 3, 4 or up to the maximum possible number of identical or different radicals selected from the group consisting of halogen, cyano, C₁-C₆-alkyl and C₁-C₆-alkoxy.
